SEQUENCE LISTING (43 sequences)

SEQ ID NO: 1 - protein, 567 aa, Influenza virus B/Washington/02/2019
DRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINAEDAPGRPYEIGTSGSCPNITNGNGFFATMAWAVPKNKTATNPLTIEVPYICTEGEDQITVWGFHSDNETQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 2 - protein, 569 aa, Influenza virus B/Singapore/INFKK-16-0569/2016
DRICTGITSSNSPHVVKTATQGEVNVTGVIPLTTTPTKSYFANLKGTRTRGKLCPDCLNCTDLDVALGRPMCVGTTPSAKASILHEVRPVTSGCFPIMHDRTKIRQLPNLLRGYEKIRLSTQNVIDAEKAPGGPYRLGTSGSCPNATSKIGFFATMAWAVPKDNYKNATNPQTVEVPYICTEGEDQITVWGFHSDNKTQMKSLYGDSNPQKFTSSANGVTTHYVSQIGDFPDQTEDGGLPQSGRIVVDYMVQKPGKTGTIVYQRGVLLPQKVWCASGRSKVIKGSLPLIGEADCLHEEYGGLNKSKPYYTGKHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVDIGNGCFETKHKCNQTCLDRIAAGTFNAGEFSLPTFDSLNITAASLNDDGLDNHTILLYYSTAASSLAVTLMLAIFIVYMVSRDNVSCSICL

SEQ ID NO: 3 - protein, 567 aa, Influenza virus B/Rhode Island/01/2019
DRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINAEDAPGGPYEIGTSGSCPNITNGKGFFATMAWAVPKNKTATNPLTIEVPYICTEEEDQITVWGFHSDDETQMAKLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 4 - protein, 567 aa, Influenza virus B/Michigan/01/2021
DRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICAEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 5 - protein, 567 aa, Influenza virus B/Henan-Xigong/1118/2021
DRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTQNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 6 - protein, 567 aa, Influenza virus B/Austria/1359417/2021
DRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 7 - protein, 567 aa, Influenza virus B/Singapore/WUH4618/2021
DRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMVRLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 8 - DNA, 69 bp, synthetic construct
  source 1..69: mol_type = other DNA
tcgtgcttcggcaccagtacaatggcgaaaaacgttgcgattttcggcttattgttttctcttcttgtg

SEQ ID NO: 9 - DNA, 46 bp, synthetic construct
  source 1..46: mol_type = other DNA
actaaagaaaataggcctttaaatacatattctacactgtagagac

SEQ ID NO: 10 - DNA, 1731 bp, synthetic construct
  source 1..1731: mol_type = other DNA
atggcgaaaaacgttgcgattttcggcttattgttttctcttcttgtgttggttccttctcagatcttcgcggatcgaatctgcactgggataacatcttcaaactcacctcatgtggtcaaaacagctactcaaggggaggtcaatgtgactggcgtgataccactgacaacaacaccaacaaaatcttattttgcaaatctcaaaggaacaaggaccagagggaaactatgcccggactgtctcaactgtacagatctggatgtggccttgggcaggccaatgtgtgtggggaccacaccttctgctaaagcttcaatactccatgaggtcagacctgttacatccgggtgctttcctataatgcacgacagaacaaaaatcaggcaactacccaatcttctcagaggatatgaaaagatcaggttatcaacccaaaacgttatcgatgcagaaaaagcaccaggaggaccctacagacttggaacctcaggatcttgccctaacgctaccagtaaaatcggattttttgcaacaatggcttgggctgtcccaaaggacaactacaaaaatgcaacgaacccacaaacagtggaagtaccatacatttgtacagaaggggaagaccaaattactgtttgggggttccattcggataacaaaacccaaatgaagagcctctatggagactcaaatcctcaaaagttcacctcatctgctaatggagtaaccacgcattatgtttctcagattggcgacttcccagatcaaacagaagacggaggactaccacaaagcggcagaattgttgttgattacatggtgcaaaaacctgggaaaacaggaacaattgtctatcaaaggggtgttttgttgcctcaaaaggtgtggtgcgcgagtggcaggagcaaagtaataaaagggtcattgcctttaattggtgaagcagattgccttcatgaagaatacggtggattaaacaaaagcaagccttactacacaggaaaacatgcaaaagccataggaaattgcccaatatgggtaaaaacacctttgaagcttgccaatggaaccaaatatagacctcctggtggaggatgggaaggaatgattgcaggttggcacggatacacatctcacggagcacatggagtggcagtggcggcagaccttaagagtacacaagaagctataaataagataacaaaaaatctcaattctttgagtgaactagaagtaaagaaccttcaaagactaagtggtgccatggatgaactccacaacgaaatactcgagctggatgaaaaagtggatgatctcagagctgacactataagctcacaaatagaacttgcagtcttgctttccaacgaaggaataataaacagtgaagacgagcatctattggcacttgagagaaaactaaagaaaatgctgggtccctctgctgtagacataggaaacggatgcttcgaaaccaaacacaaatgcaaccagacctgcttagacaggatagctgctggcacctttaatgcaggagaattttctctccccacttttgattcattgaacattactgctgcatctttaaatgatgatggattggataactaccagattttggcgatctattcaactgtcgccagttcattggtactggtagtctccctgggggcaatcagtttctggatgtgctctaatgggtctctacagtgtagaatatgtatttaa

SEQ ID NO: 11 - protein, 576 aa, synthetic construct
MAKNVAIFGLLFSLLVLVPSQIFADRICTGITSSNSPHVVKTATQGEVNVTGVIPLTTTPTKSYFANLKGTRTRGKLCPDCLNCTDLDVALGRPMCVGTTPSAKASILHEVRPVTSGCFPIMHDRTKIRQLPNLLRGYEKIRLSTQNVIDAEKAPGGPYRLGTSGSCPNATSKIGFFATMAWAVPKDNYKNATNPQTVEVPYICTEGEDQITVWGFHSDNKTQMKSLYGDSNPQKFTSSANGVTTHYVSQIGDFPDQTEDGGLPQSGRIVVDYMVQKPGKTGTIVYQRGVLLPQKVWCASGRSKVIKGSLPLIGEADCLHEEYGGLNKSKPYYTGKHAKAIGNCPIWVKTPLKLANGTKYRPPGGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVDIGNGCFETKHKCNQTCLDRIAAGTFNAGEFSLPTFDSLNITAASLNDDGLDNYQILAIYSTVASSLVLVVSLGAISFWMCSNGSLQCRICI

SEQ ID NO: 12 - DNA, 1731 bp, synthetic construct
  source 1..1731: mol_type = other DNA
atggcgaaaaacgttgcgattttcggcttattgttttctcttcttgtgttggttccttctcagatcttcgcggatcgaatctgcactgggataacatcttcaaactcacctcatgtggtcaaaacagctactcaaggggaggtcaatgtgactggcgtgataccactgacaacaacaccaacaaaatcttattttgcaaatctcaaaggaacaaggaccagagggaaactatgcccggactgtctcaactgtacagatctggatgtggccttgggcaggccaatgtgtgtggggaccacaccttctgctaaagcttcaatactccatgaggtcagacctgttacatccgggtgctttcctataatgcacgacagaacaaaaatcaggcaactacccaatcttctcagaggatatgaaaagatcaggttatcaacccaaaacgttatcgatgcagaaaaagcaccaggaggaccctacagacttggaacctcaggatcttgccctaacgctaccagtaaaatcggattttttgcaacaatggcttgggctgtcccaaaggacaactacaaaaatgcaacgaacccacaaacagtggaagtaccatacatttgtacagaaggggaagaccaaattactgtttgggggttccattcggataacaaaacccaaatgaagagcctctatggagactcaaatcctcaaaagttcacctcatctgctaatggagtaaccacgcattatgtttctcagattggcgacttcccagatcaaacagaagacggaggactaccacaaagcggcagaattgttgttgattacatggtgcaaaaacctgggaaaacaggaacaattgtctatcaaaggggtgttttgttgcctcaaaaggtgtggtgcgcgagtggcaggagcaaagtaataaaagggtcattgcctttaattggtgaagcagattgccttcatgaagaatacggtggattaaacaaaagcaagccttactacacaggaaaacatgcaaaagccataggaaattgcccaatatgggtaaaaacacctttgaagcttgccaatggaaccaaatatagacctcctggtggaggatgggaaggaatgattgcaggttggcacggatacacatctcacggagcacatggagtggcagtggcggcagaccttaagagtacacaagaagctataaataagataacaaaaaatctcaattctttgagtgaaatcgaagtaaagaaccttcaaagactaagtggtgccatggatgaactccacaacgaaatactcgagctggatgaaaaagtggatgatctcagagctgacactataagctcacaaatagaacttgcagtcttgctttccaacgaaggaataataaacagtgaagacgagcatctattggcacttgagagaaaactaaagaaaatgctgggtccctctgctgtagacataggaaacggatgcttcgaaaccaaacacaaatgcaaccagacctgcttagacaggatagctgctggcacctttaatgcaggagaattttctctccccacttttgattcattgaacattactgctgcatctttaaatgatgatggattggataactaccagattttggcgatctattcaactgtcgccagttcattggtactggtagtctccctgggggcaatcagtttctggatgtgctctaatgggtctctacagtgtagaatatgtatttaa

SEQ ID NO: 13 - protein, 576 aa, synthetic construct
MAKNVAIFGLLFSLLVLVPSQIFADRICTGITSSNSPHVVKTATQGEVNVTGVIPLTTTPTKSYFANLKGTRTRGKLCPDCLNCTDLDVALGRPMCVGTTPSAKASILHEVRPVTSGCFPIMHDRTKIRQLPNLLRGYEKIRLSTQNVIDAEKAPGGPYRLGTSGSCPNATSKIGFFATMAWAVPKDNYKNATNPQTVEVPYICTEGEDQITVWGFHSDNKTQMKSLYGDSNPQKFTSSANGVTTHYVSQIGDFPDQTEDGGLPQSGRIVVDYMVQKPGKTGTIVYQRGVLLPQKVWCASGRSKVIKGSLPLIGEADCLHEEYGGLNKSKPYYTGKHAKAIGNCPIWVKTPLKLANGTKYRPPGGGWEGMIAGWHGYTSHGAHGVAVAADLKSTQEAINKITKNLNSLSEIEVKNLQRLSGAMDELHNEILELDEKVDDLRADTISSQIELAVLLSNEGIINSEDEHLLALERKLKKMLGPSAVDIGNGCFETKHKCNQTCLDRIAAGTFNAGEFSLPTFDSLNITAASLNDDGLDNYQILAIYSTVASSLVLVVSLGAISFWMCSNGSLQCRICI

SEQ ID NO: 14 - DNA, 1725 bp, synthetic construct
  source 1..1725: mol_type = other DNA
atggcgaaaaacgttgcgattttcggcttattgttttctcttcttgtgttggttccttctcagatcttcgcggatcgaatctgcactgggataacatcgtcaaactcaccacatgtcgtcaaaactgctactcaaggggaggtcaatgtgaccggtgtaataccactgacaacaacacccaccaaatctcattttgcaaatctcaaaggaacagaaaccagggggaaactatgcccaaaatgcctcaactgcacagatctggatgtagccttgggcagaccaaaatgcacagggaaaataccctctgcaagggtttcaatactccatgaagtcagacctgttacatctgggtgctttcctataatgcacgatagaacaaaaattagacagctgcctaaccttctccgaggatacgaacatgtcaggttatcaactcacaacgttatcaatgcagaagatgcaccaggaagaccctacgagattggaacctcagggtcttgccctaacattaccaatggaaacggattcttcgcaacaatggcttgggccgtcccaaaaaacaaaacagcaacaaatccattaacaatagaagtaccatacatttgtacagaaggagaagaccaaattaccgtttgggggttccactctgacaacgagacccaaatggcaaagctctatggggactcaaagccccagaagttcacctcatctgccaacggagtgaccacacattacgtttcacagattggtggcttcccaaatcaaacagaagacggaggactaccacaaagtggcagaattgttgttgattacatggtgcaaaaatctggaaaaacaggaacaattacctatcaaagaggtattttattgcctcaaaaggtgtggtgcgcaagtggcaggagcaaggtaataaaaggatccttgcctttaattggagaagcagattgcctccatgaaaaatacggtggattaaacaaaagcaagccttactacacaggggaacatgcaaaggccataggaaattgcccaatatgggtgaaaacacccttgaagctggccaatggaaccaaatatagacctcctggtggaggatgggaaggaatgattgcaggttggcacggatacacatcccatggggcacatggagtagcggtggcagcagaccttaagagcactcaagaggccataaacaagataacaaaaaatctcaactctttgagtgagctggaagtaaagaatcttcaaagactaagcggtgccatggatgaactccacaacgaaatactagaactagatgagaaagtggatgatctcagagctgatacaataagctcacaaatagaactcgcagtcctgctttccaatgaaggaataataaacagtgaagatgaacatctcttggcgcttgaaagaaagctgaagaaaatgctgggcccctctgctgtagagatagggaatggatgctttgaaaccaaacacaagtgcaaccagacctgtctcgacagaatagctgctggtacctttgatgcaggagaattttctctccccacctttgattcactgaatattactgctgcatctttaaatgacgatggattggataattaccagattttggcgatctattcaactgtcgccagttcattggtactggtagtctccctgggggcaatcagtttctggatgtgctctaatgggtctctacagtgtagaatatgtatttaa

SEQ ID NO: 15 - protein, 574 aa, synthetic construct
MAKNVAIFGLLFSLLVLVPSQIFADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINAEDAPGRPYEIGTSGSCPNITNGNGFFATMAWAVPKNKTATNPLTIEVPYICTEGEDQITVWGFHSDNETQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPGGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNYQILAIYSTVASSLVLVVSLGAISFWMCSNGSLQCRICI

SEQ ID NO: 16 - DNA, 1725 bp, synthetic construct
  source 1..1725: mol_type = other DNA
atggcgaaaaacgttgcgattttcggcttattgttttctcttcttgtgttggttccttctcagatcttcgcggatcgaatctgcactgggataacatcgtcaaactcaccacatgtcgtcaaaactgctactcaaggggaggtcaatgtgaccggtgtaataccactgacaacaacacccaccaaatctcattttgcaaatctcaaaggaacagaaaccagggggaaactatgcccaaaatgcctcaactgcacagatctggatgtagccttgggcagaccaaaatgcacagggaaaataccctctgcaagggtttcaatactccatgaagtcagacctgttacatctgggtgctttcctataatgcacgatagaacaaaaattagacagctgcctaaccttctccgaggatacgaacatgtcaggttatcaactcacaacgttatcaatgcagaagatgcaccaggaagaccctacgagattggaacctcagggtcttgccctaacattaccaatggaaacggattcttcgcaacaatggcttgggccgtcccaaaaaacaaaacagcaacaaatccattaacaatagaagtaccatacatttgtacagaaggagaagaccaaattaccgtttgggggttccactctgacaacgagacccaaatggcaaagctctatggggactcaaagccccagaagttcacctcatctgccaacggagtgaccacacattacgtttcacagattggtggcttcccaaatcaaacagaagacggaggactaccacaaagtggcagaattgttgttgattacatggtgcaaaaatctggaaaaacaggaacaattacctatcaaagaggtattttattgcctcaaaaggtgtggtgcgcaagtggcaggagcaaggtaataaaaggatccttgcctttaattggagaagcagattgcctccatgaaaaatacggtggattaaacaaaagcaagccttactacacaggggaacatgcaaaggccataggaaattgcccaatatgggtgaaaacacccttgaagctggccaatggaaccaaatatagacctcctggtggaggatgggaaggaatgattgcaggttggcacggatacacatcccatggggcacatggagtagcggtggcagcagaccttaagagcactcaagaggccataaacaagataacaaaaaatctcaactctttgagtgagatcgaagtaaagaatcttcaaagactaagcggtgccatggatgaactccacaacgaaatactagaactagatgagaaagtggatgatctcagagctgatacaataagctcacaaatagaactcgcagtcctgctttccaatgaaggaataataaacagtgaagatgaacatctcttggcgcttgaaagaaagctgaagaaaatgctgggcccctctgctgtagagatagggaatggatgctttgaaaccaaacacaagtgcaaccagacctgtctcgacagaatagctgctggtacctttgatgcaggagaattttctctccccacctttgattcactgaatattactgctgcatctttaaatgacgatggattggataattaccagattttggcgatctattcaactgtcgccagttcattggtactggtagtctccctgggggcaatcagtttctggatgtgctctaatgggtctctacagtgtagaatatgtatttaa

SEQ ID NO: 17 - protein, 574 aa, synthetic construct
MAKNVAIFGLLFSLLVLVPSQIFADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINAEDAPGRPYEIGTSGSCPNITNGNGFFATMAWAVPKNKTATNPLTIEVPYICTEGEDQITVWGFHSDNETQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPGGGWEGMIAGWHGYTSHGAHGVAVAADLKSTQEAINKITKNLNSLSEIEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNYQILAIYSTVASSLVLVVSLGAISFWMCSNGSLQCRICI

SEQ ID NO: 18 - DNA, 1725 bp, synthetic construct
  source 1..1725: mol_type = other DNA
atggcgaaaaacgttgcgattttcggcttattgttttctcttcttgtgttggttccttctcagatcttcgcggatcgaatctgcactgggataacatcgtcaaactcaccacatgtcgtcaaaactgctactcaaggggaggtcaatgtgaccggtgtaataccactgacaacaacacccaccaaatctcattttgcaaatctcaaaggaacagaaaccagggggaaactatgcccaaaatgcctcaactgcacagatctggatgtagccttgggcagaccaaaatgcacagggaaaataccctctgcaagggtttcaatactccatgaagtcagacctgttacatctgggtgctttcctataatgcacgatagaacaaaaattagacagctgcctaaccttctccgaggatacgaacatgtcaggttatcaactcacaacgttatcaatgcagaagatgcaccaggaggaccctacgagattggaacctcagggtcttgccctaacattaccaatggaaagggattcttcgcaacaatggcttgggccgtcccaaaaaacaaaacagcaacaaatccattaacaatagaagtaccatacatttgtacagaagaggaagaccaaattaccgtttgggggttccactctgacgacgagacccaaatggcaaagctctatggggactcaaagccccagaagttcacctcatctgccaacggagtgaccacacattacgtttcacagattggtggcttcccaaatcaaacagaagacggaggactaccacaaagtggcagaattgttgttgattacatggtgcaaaaatctggaaaaacaggaacaattacctatcaaagaggtattttattgcctcaaaaggtgtggtgcgcaagtggcaagagcaaggtaataaaaggatccttgcctttaattggagaagcagattgcctccatgaaaaatacggtggattaaacaaaagcaagccttactacacaggggaacatgcaaaggccataggaaattgcccaatatgggtgaaaacacccttgaagctggccaatggaaccaaatatagacctcctggtggaggatgggaaggaatgattgcaggttggcacggatacacatcccatggggcacatggagtagcggtggcagcagaccttaagagcactcaagaggccataaacaagataacaaaaaatctcaactctttgagtgagctggaagtaaagaatcttcaaagactaagcggtgccatggatgaactccacaacgaaatactagaactagatgagaaagtggatgatctcagagctgatacaataagctcacaaatagaactcgcagtcctgctttccaatgaaggaataataaacagtgaagatgaacatctcttggcgcttgaaagaaagctgaagaaaatgctgggcccctctgctgtagagatagggaatggatgctttgaaaccaaacacaagtgcaaccagacctgtctcgacagaatagctgctggtacctttgatgcaggagaattttctctccccacctttgattcactgaatattactgctgcatctttaaatgacgatggattggataattaccagattttggcgatctattcaactgtcgccagttcattggtactggtagtctccctgggggcaatcagtttctggatgtgctctaatgggtctctacagtgtagaatatgtatttaa

SEQ ID NO: 19 - protein, 574 aa, synthetic construct
MAKNVAIFGLLFSLLVLVPSQIFADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINAEDAPGGPYEIGTSGSCPNITNGKGFFATMAWAVPKNKTATNPLTIEVPYICTEEEDQITVWGFHSDDETQMAKLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPGGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNYQILAIYSTVASSLVLVVSLGAISFWMCSNGSLQCRICI

SEQ ID NO: 20 - DNA, 1725 bp, synthetic construct
  source 1..1725: mol_type = other DNA
atggcgaaaaacgttgcgattttcggcttattgttttctcttcttgtgttggttccttctcagatcttcgcggatcgaatctgcactgggataacatcgtcaaactcaccacatgtcgtcaaaactgctactcaaggggaggtcaatgtgaccggtgtaataccactgacaacaacacccaccaaatctcattttgcaaatctcaaaggaacagaaaccagggggaaactatgcccaaaatgcctcaactgcacagatctggatgtagccttgggcagaccaaaatgcacagggaaaataccctctgcaagggtttcaatactccatgaagtcagacctgttacatctgggtgctttcctataatgcacgatagaacaaaaattagacagctgcctaaccttctccgaggatacgaacatgtcaggttatcaactcacaacgttatcaatgcagaagatgcaccaggaggaccctacgagattggaacctcagggtcttgccctaacattaccaatggaaagggattcttcgcaacaatggcttgggccgtcccaaaaaacaaaacagcaacaaatccattaacaatagaagtaccatacatttgtacagaagaggaagaccaaattaccgtttgggggttccactctgacgacgagacccaaatggcaaagctctatggggactcaaagccccagaagttcacctcatctgccaacggagtgaccacacattacgtttcacagattggtggcttcccaaatcaaacagaagacggaggactaccacaaagtggcagaattgttgttgattacatggtgcaaaaatctggaaaaacaggaacaattacctatcaaagaggtattttattgcctcaaaaggtgtggtgcgcaagtggcaagagcaaggtaataaaaggatccttgcctttaattggagaagcagattgcctccatgaaaaatacggtggattaaacaaaagcaagccttactacacaggggaacatgcaaaggccataggaaattgcccaatatgggtgaaaacacccttgaagctggccaatggaaccaaatatagacctcctggtggaggatgggaaggaatgattgcaggttggcacggatacacatcccatggggcacatggagtagcggtggcagcagaccttaagagcactcaagaggccataaacaagataacaaaaaatctcaactctttgagtgagatcgaagtaaagaatcttcaaagactaagcggtgccatggatgaactccacaacgaaatactagaactagatgagaaagtggatgatctcagagctgatacaataagctcacaaatagaactcgcagtcctgctttccaatgaaggaataataaacagtgaagatgaacatctcttggcgcttgaaagaaagctgaagaaaatgctgggcccctctgctgtagagatagggaatggatgctttgaaaccaaacacaagtgcaaccagacctgtctcgacagaatagctgctggtacctttgatgcaggagaattttctctccccacctttgattcactgaatattactgctgcatctttaaatgacgatggattggataattaccagattttggcgatctattcaactgtcgccagttcattggtactggtagtctccctgggggcaatcagtttctggatgtgctctaatgggtctctacagtgtagaatatgtatttaa

SEQ ID NO: 21 - protein, 574 aa, synthetic construct
MAKNVAIFGLLFSLLVLVPSQIFADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINAEDAPGGPYEIGTSGSCPNITNGKGFFATMAWAVPKNKTATNPLTIEVPYICTEEEDQITVWGFHSDDETQMAKLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPGGGWEGMIAGWHGYTSHGAHGVAVAADLKSTQEAINKITKNLNSLSEIEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNYQILAIYSTVASSLVLVVSLGAISFWMCSNGSLQCRICI

SEQ ID NO: 22 - DNA, 1725 bp, synthetic construct
  source 1..1725: mol_type = other DNA
atggcgaaaaacgttgcgattttcggcttattgttttctcttcttgtgttggttccttctcagatcttcgcggatcgaatctgcactgggataacatcgtcaaactcaccacatgtcgtcaaaactgctactcaaggggaggtcaatgtgaccggtgtaataccactgacaacaacacccaccaaatctcattttgcaaatctcaaaggaacagaaaccagggggaaactatgcccaaaatgcctcaactgcacagatctggatgtagccttgggcagaccaaaatgcacagggaaaataccctctgcaagggtttcaatactccatgaagtcagacctgttacatctgggtgctttcctataatgcacgatagaacaaaaattagacagctgcctaaccttctccgaggatacgaacatgtcaggttatcaactcacaacgttatcaataccgaagatgcaccaggaggaccctacgagattggaacctcagggtcttgcctgaacattaccaatggaaagggattcttcgcaacaatggcttgggccgtcccaaaaaacaaaacagcaacaaatccattaacaatagaagtaccatacatttgtgccgaagaggaagaccaaattaccgtttgggggttccactctgacgacgagacccaaatggcaagactctatggggactcaaagccccagaagttcacctcatctgccaacggagtgaccacacattacgtttcacagattggtggcttcccaaatcaaacagaagacggaggactaccacaaagtggcagaattgttgttgattacatggtgcaaaaatctggaaaaacaggaacaattacctatcaaagaggtattttattgcctcaaaaggtgtggtgcgcaagtggcaagagcaaggtaataaaaggatccttgcctttaattggagaagcagattgcctccatgaaaaatacggtggattaaacaaaagcaagccttactacacaggggaacatgcaaaggccataggaaattgcccaatatgggtgaaaacacccttgaagctggccaatggaaccaaatatagacctcctggtggaggatgggaaggaatgattgcaggttggcacggatacacatcccatggggcacatggagtagcggtggcagcagaccttaagagcactcaagaggccataaacaagataacaaaaaatctcaactctttgagtgagctggaagtaaagaatcttcaaagactaagcggtgccatggatgaactccacaacgaaatactagaactagatgagaaagtggatgatctcagagctgatacaataagctcacaaatagaactcgcagtcctgctttccaatgaaggaataataaacagtgaagatgaacatctcttggcgcttgaaagaaagctgaagaaaatgctgggcccctctgctgtagagatagggaatggatgctttgaaaccaaacacaagtgcaaccagacctgtctcgacagaatagctgctggtacctttgatgcaggagaattttctctccccacctttgattcactgaatattactgctgcatctttaaatgacgatggattggataattaccagattttggcgatctattcaactgtcgccagttcattggtactggtagtctccctgggggcaatcagtttctggatgtgctctaatgggtctctacagtgtagaatatgtatttaa

SEQ ID NO: 23 - protein, 574 aa, synthetic construct
MAKNVAIFGLLFSLLVLVPSQIFADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICAEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPGGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNYQILAIYSTVASSLVLVVSLGAISFWMCSNGSLQCRICI

SEQ ID NO: 24 - DNA, 1725 bp, synthetic construct
  source 1..1725: mol_type = other DNA
atggcgaaaaacgttgcgattttcggcttattgttttctcttcttgtgttggttccttctcagatcttcgcggatcgaatctgcactgggataacatcgtcaaactcaccacatgtcgtcaaaactgctactcaaggggaggtcaatgtgaccggtgtaataccactgacaacaacacccaccaaatctcattttgcaaatctcaaaggaacagaaaccagggggaaactatgcccaaaatgcctcaactgcacagatctggatgtagccttgggcagaccaaaatgcacagggaaaataccctctgcaagggtttcaatactccatgaagtcagacctgttacatctgggtgctttcctataatgcacgatagaacaaaaattagacagctgcctaaccttctccgaggatacgaacatgtcaggttatcaactcacaacgttatcaataccgaagatgcaccaggaggaccctacgagattggaacctcagggtcttgcctgaacattaccaatggaaagggattcttcgcaacaatggcttgggccgtcccaaaaaacaaaacagcaacaaatccattaacaatagaagtaccatacatttgtgccgaagaggaagaccaaattaccgtttgggggttccactctgacgacgagacccaaatggcaagactctatggggactcaaagccccagaagttcacctcatctgccaacggagtgaccacacattacgtttcacagattggtggcttcccaaatcaaacagaagacggaggactaccacaaagtggcagaattgttgttgattacatggtgcaaaaatctggaaaaacaggaacaattacctatcaaagaggtattttattgcctcaaaaggtgtggtgcgcaagtggcaagagcaaggtaataaaaggatccttgcctttaattggagaagcagattgcctccatgaaaaatacggtggattaaacaaaagcaagccttactacacaggggaacatgcaaaggccataggaaattgcccaatatgggtgaaaacacccttgaagctggccaatggaaccaaatatagacctcctggtggaggatgggaaggaatgattgcaggttggcacggatacacatcccatggggcacatggagtagcggtggcagcagaccttaagagcactcaagaggccataaacaagataacaaaaaatctcaactctttgagtgagatcgaagtaaagaatcttcaaagactaagcggtgccatggatgaactccacaacgaaatactagaactagatgagaaagtggatgatctcagagctgatacaataagctcacaaatagaactcgcagtcctgctttccaatgaaggaataataaacagtgaagatgaacatctcttggcgcttgaaagaaagctgaagaaaatgctgggcccctctgctgtagagatagggaatggatgctttgaaaccaaacacaagtgcaaccagacctgtctcgacagaatagctgctggtacctttgatgcaggagaattttctctccccacctttgattcactgaatattactgctgcatctttaaatgacgatggattggataattaccagattttggcgatctattcaactgtcgccagttcattggtactggtagtctccctgggggcaatcagtttctggatgtgctctaatgggtctctacagtgtagaatatgtatttaa

SEQ ID NO: 25 - protein, 574 aa, synthetic construct
MAKNVAIFGLLFSLLVLVPSQIFADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICAEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPGGGWEGMIAGWHGYTSHGAHGVAVAADLKSTQEAINKITKNLNSLSEIEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNYQILAIYSTVASSLVLVVSLGAISFWMCSNGSLQCRICI

SEQ ID NO: 26 - DNA, 1725 bp, synthetic construct
  source 1..1725: mol_type = other DNA
atggcgaaaaacgttgcgattttcggcttattgttttctcttcttgtgttggttccttctcagatcttcgcggatcgaatctgcactgggataacatcgtcaaactcaccacatgtcgtcaaaactgctactcaaggggaggtcaatgtgaccggtgtaataccactgacaacaacacccaccaaatctcattttgcaaatctcaaaggaacagaaaccagggggaaactatgcccaaaatgcctcaactgcacagatctggatgtagccttgggcagaccaaaatgcacagggaaaataccctctgcaagggtttcaatactccatgaagtcagacctgttacatctgggtgctttcctataatgcacgatagaacaaaaattagacagctgcctaaccttctccgaggatacgaacatgtcaggttatcaactcagaacgttatcaataccgaagatgcaccaggaggaccctacgagattggaacctcagggtcttgcctgaacattaccaatggaaagggattcttcgcaacaatggcttgggccgtcccaaaaaacaaaacagcaacaaatccattaacaatagaagtaccatacatttgtacagaagaggaagaccaaattaccgtttgggggttccactctgacgacgagacccaaatggcaagactctatggggactcaaagccccagaagttcacctcatctgccaacggagtgaccacacattacgtttcacagattggtggcttcccaaatcaaacagaagacggaggactaccacaaagtggcagaattgttgttgattacatggtgcaaaaatctggaaaaacaggaacaattacctatcaaagaggtattttattgcctcaaaaggtgtggtgcgcaagtggcaagagcaaggtaataaaaggatccttgcctttaattggagaagcagattgcctccatgaaaaatacggtggattaaacaaaagcaagccttactacacaggggaacatgcaaaggccataggaaattgcccaatatgggtgaaaacacccttgaagctggccaatggaaccaaatatagacctcctggtggaggatgggaaggaatgattgcaggttggcacggatacacatcccatggggcacatggagtagcggtggcagcagaccttaagagcactcaagaggccataaacaagataacaaaaaatctcaactctttgagtgagctggaagtaaagaatcttcaaagactaagcggtgccatggatgaactccacaacgaaatactagaactagatgagaaagtggatgatctcagagctgatacaataagctcacaaatagaactcgcagtcctgctttccaatgaaggaataataaacagtgaagatgaacatctcttggcgcttgaaagaaagctgaagaaaatgctgggcccctctgctgtagagatagggaatggatgctttgaaaccaaacacaagtgcaaccagacctgtctcgacagaatagctgctggtacctttgatgcaggagaattttctctccccacctttgattcactgaatattactgctgcatctttaaatgacgatggattggataattaccagattttggcgatctattcaactgtcgccagttcattggtactggtagtctccctgggggcaatcagtttctggatgtgctctaatgggtctctacagtgtagaatatgtatttaa

SEQ ID NO: 27 - protein, 574 aa, synthetic construct
MAKNVAIFGLLFSLLVLVPSQIFADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTQNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPGGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNYQILAIYSTVASSLVLVVSLGAISFWMCSNGSLQCRICI

SEQ ID NO: 28 - DNA, 1725 bp, synthetic construct
  source 1..1725: mol_type = other DNA
atggcgaaaaacgttgcgattttcggcttattgttttctcttcttgtgttggttccttctcagatcttcgcggatcgaatctgcactgggataacatcgtcaaactcaccacatgtcgtcaaaactgctactcaaggggaggtcaatgtgaccggtgtaataccactgacaacaacacccaccaaatctcattttgcaaatctcaaaggaacagaaaccagggggaaactatgcccaaaatgcctcaactgcacagatctggatgtagccttgggcagaccaaaatgcacagggaaaataccctctgcaagggtttcaatactccatgaagtcagacctgttacatctgggtgctttcctataatgcacgatagaacaaaaattagacagctgcctaaccttctccgaggatacgaacatgtcaggttatcaactcagaacgttatcaataccgaagatgcaccaggaggaccctacgagattggaacctcagggtcttgcctgaacattaccaatggaaagggattcttcgcaacaatggcttgggccgtcccaaaaaacaaaacagcaacaaatccattaacaatagaagtaccatacatttgtacagaagaggaagaccaaattaccgtttgggggttccactctgacgacgagacccaaatggcaagactctatggggactcaaagccccagaagttcacctcatctgccaacggagtgaccacacattacgtttcacagattggtggcttcccaaatcaaacagaagacggaggactaccacaaagtggcagaattgttgttgattacatggtgcaaaaatctggaaaaacaggaacaattacctatcaaagaggtattttattgcctcaaaaggtgtggtgcgcaagtggcaagagcaaggtaataaaaggatccttgcctttaattggagaagcagattgcctccatgaaaaatacggtggattaaacaaaagcaagccttactacacaggggaacatgcaaaggccataggaaattgcccaatatgggtgaaaacacccttgaagctggccaatggaaccaaatatagacctcctggtggaggatgggaaggaatgattgcaggttggcacggatacacatcccatggggcacatggagtagcggtggcagcagaccttaagagcactcaagaggccataaacaagataacaaaaaatctcaactctttgagtgagatcgaagtaaagaatcttcaaagactaagcggtgccatggatgaactccacaacgaaatactagaactagatgagaaagtggatgatctcagagctgatacaataagctcacaaatagaactcgcagtcctgctttccaatgaaggaataataaacagtgaagatgaacatctcttggcgcttgaaagaaagctgaagaaaatgctgggcccctctgctgtagagatagggaatggatgctttgaaaccaaacacaagtgcaaccagacctgtctcgacagaatagctgctggtacctttgatgcaggagaattttctctccccacctttgattcactgaatattactgctgcatctttaaatgacgatggattggataattaccagattttggcgatctattcaactgtcgccagttcattggtactggtagtctccctgggggcaatcagtttctggatgtgctctaatgggtctctacagtgtagaatatgtatttaa

SEQ ID NO: 29 - protein, 574 aa, synthetic construct
MAKNVAIFGLLFSLLVLVPSQIFADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTQNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPGGGWEGMIAGWHGYTSHGAHGVAVAADLKSTQEAINKITKNLNSLSEIEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNYQILAIYSTVASSLVLVVSLGAISFWMCSNGSLQCRICI

SEQ ID NO: 30 - DNA, 1725 bp, synthetic construct
  source 1..1725: mol_type = other DNA
atggcgaaaaacgttgcgattttcggcttattgttttctcttcttgtgttggttccttctcagatcttcgcggatcgaatctgcactgggataacatcgtcaaactcaccacatgtcgtcaaaactgctactcaaggggaggtcaatgtgaccggtgtaataccactgacaacaacacccaccaaatctcattttgcaaatctcaaaggaacagaaaccagggggaaactatgcccaaaatgcctcaactgcacagatctggatgtagccttgggcagaccaaaatgcacagggaaaataccctctgcaagggtttcaatactccatgaagtcagacctgttacatctgggtgctttcctataatgcacgatagaacaaaaattagacagctgcctaaccttctccgaggatacgaacatgtcaggttatcaactcacaacgttatcaataccgaagatgcaccaggaggaccctacgagattggaacctcagggtcttgcctgaacattaccaatggaaagggattcttcgcaacaatggcttgggccgtcccaaaaaacaaaacagcaacaaatccattaacaatagaagtaccatacatttgtaccgaagaggaagaccaaattaccgtttgggggttccactctgacgacgagacccaaatggtgagactctatggggactcaaagccccagaagttcacctcatctgccaacggagtgaccacacattacgtttcacagattggtggcttcccaaatcaaacagaagacggaggactaccacaaagtggcagaattgttgttgattacatggtgcaaaaatctggaaaaacaggaacaattacctatcaaagaggtattttattgcctcaaaaggtgtggtgcgcaagtggcaagagcaaggtaataaaaggatccttgcctttaattggagaagcagattgcctccatgaaaaatacggtggattaaacaaaagcaagccttactacacaggggaacatgcaaaggccataggaaattgcccaatatgggtgaaaacacccttgaagctggccaatggaaccaaatatagacctcctggtggaggatgggaaggaatgattgcaggttggcacggatacacatcccatggggcacatggagtagcggtggcagcagaccttaagagcactcaagaggccataaacaagataacaaaaaatctcaactctttgagtgagctggaagtaaagaatcttcaaagactaagcggtgccatggatgaactccacaacgaaatactagaactagatgagaaagtggatgatctcagagctgatacaataagctcacaaatagaactcgcagtcctgctttccaatgaaggaataataaacagtgaagatgaacatctcttggcgcttgaaagaaagctgaagaaaatgctgggcccctctgctgtagagatagggaatggatgctttgaaaccaaacacaagtgcaaccagacctgtctcgacagaatagctgctggtacctttgatgcaggagaattttctctccccacctttgattcactgaatattactgctgcatctttaaatgacgatggattggataattaccagattttggcgatctattcaactgtcgccagttcattggtactggtagtctccctgggggcaatcagtttctggatgtgctctaatgggtctctacagtgtagaatatgtatttaa

SEQ ID NO: 31 - protein, 574 aa, synthetic construct
MAKNVAIFGLLFSLLVLVPSQIFADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMVRLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPGGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNYQILAIYSTVASSLVLVVSLGAISFWMCSNGSLQCRICI

SEQ ID NO: 32 - DNA, 1725 bp, synthetic construct
  source 1..1725: mol_type = other DNA
atggcgaaaaacgttgcgattttcggcttattgttttctcttcttgtgttggttccttctcagatcttcgcggatcgaatctgcactgggataacatcgtcaaactcaccacatgtcgtcaaaactgctactcaaggggaggtcaatgtgaccggtgtaataccactgacaacaacacccaccaaatctcattttgcaaatctcaaaggaacagaaaccagggggaaactatgcccaaaatgcctcaactgcacagatctggatgtagccttgggcagaccaaaatgcacagggaaaataccctctgcaagggtttcaatactccatgaagtcagacctgttacatctgggtgctttcctataatgcacgatagaacaaaaattagacagctgcctaaccttctccgaggatacgaacatgtcaggttatcaactcacaacgttatcaataccgaagatgcaccaggaggaccctacgagattggaacctcagggtcttgcctgaacattaccaatggaaagggattcttcgcaacaatggcttgggccgtcccaaaaaacaaaacagcaacaaatccattaacaatagaagtaccatacatttgtaccgaagaggaagaccaaattaccgtttgggggttccactctgacgacgagacccaaatggtgagactctatggggactcaaagccccagaagttcacctcatctgccaacggagtgaccacacattacgtttcacagattggtggcttcccaaatcaaacagaagacggaggactaccacaaagtggcagaattgttgttgattacatggtgcaaaaatctggaaaaacaggaacaattacctatcaaagaggtattttattgcctcaaaaggtgtggtgcgcaagtggcaagagcaaggtaataaaaggatccttgcctttaattggagaagcagattgcctccatgaaaaatacggtggattaaacaaaagcaagccttactacacaggggaacatgcaaaggccataggaaattgcccaatatgggtgaaaacacccttgaagctggccaatggaaccaaatatagacctcctggtggaggatgggaaggaatgattgcaggttggcacggatacacatcccatggggcacatggagtagcggtggcagcagaccttaagagcactcaagaggccataaacaagataacaaaaaatctcaactctttgagtgagatcgaagtaaagaatcttcaaagactaagcggtgccatggatgaactccacaacgaaatactagaactagatgagaaagtggatgatctcagagctgatacaataagctcacaaatagaactcgcagtcctgctttccaatgaaggaataataaacagtgaagatgaacatctcttggcgcttgaaagaaagctgaagaaaatgctgggcccctctgctgtagagatagggaatggatgctttgaaaccaaacacaagtgcaaccagacctgtctcgacagaatagctgctggtacctttgatgcaggagaattttctctccccacctttgattcactgaatattactgctgcatctttaaatgacgatggattggataattaccagattttggcgatctattcaactgtcgccagttcattggtactggtagtctccctgggggcaatcagtttctggatgtgctctaatgggtctctacagtgtagaatatgtatttaa

SEQ ID NO: 33 - protein, 574 aa, synthetic construct
MAKNVAIFGLLFSLLVLVPSQIFADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMVRLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPGGGWEGMIAGWHGYTSHGAHGVAVAADLKSTQEAINKITKNLNSLSEIEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNYQILAIYSTVASSLVLVVSLGAISFWMCSNGSLQCRICI

SEQ ID NO: 34 - DNA, 1725 bp, synthetic construct
  source 1..1725: mol_type = other DNA
atggcgaaaaacgttgcgattttcggcttattgttttctcttcttgtgttggttccttctcagatcttcgcggatcgaatctgcactgggataacatcgtcaaactcaccacatgtcgtcaaaactgctactcaaggggaggtcaatgtgaccggtgtaataccactgacaacaacacccaccaaatctcattttgcaaatctcaaaggaacagaaaccagggggaaactatgcccaaaatgcctcaactgcacagatctggatgtagccttgggcagaccaaaatgcacagggaaaataccctctgcaagggtttcaatactccatgaagtcagacctgttacatctgggtgctttcctataatgcacgatagaacaaaaattagacagctgcctaaccttctccgaggatacgaacatgtcaggttatcaactcacaacgttatcaataccgaagatgcaccaggaggaccctacgagattggaacctcagggtcttgcctgaacattaccaatggaaagggattcttcgcaacaatggcttgggccgtcccaaaaaacaaaacagcaacaaatccattaacaatagaagtaccatacatttgtaccgaagaggaagaccaaattaccgtttgggggttccactctgacgacgagacccaaatggcaagactctatggggactcaaagccccagaagttcacctcatctgccaacggagtgaccacacattacgtttcacagattggtggcttcccaaatcaaacagaagacggaggactaccacaaagtggcagaattgttgttgattacatggtgcaaaaatctggaaaaacaggaacaattacctatcaaagaggtattttattgcctcaaaaggtgtggtgcgcaagtggcaagagcaaggtaataaaaggatccttgcctttaattggagaagcagattgcctccatgaaaaatacggtggattaaacaaaagcaagccttactacacaggggaacatgcaaaggccataggaaattgcccaatatgggtgaaaacacccttgaagctggccaatggaaccaaatatagacctcctggtggaggatgggaaggaatgattgcaggttggcacggatacacatcccatggggcacatggagtagcggtggcagcagaccttaagagcactcaagaggccataaacaagataacaaaaaatctcaactctttgagtgagctggaagtaaagaatcttcaaagactaagcggtgccatggatgaactccacaacgaaatactagaactagatgagaaagtggatgatctcagagctgatacaataagctcacaaatagaactcgcagtcctgctttccaatgaaggaataataaacagtgaagatgaacatctcttggcgcttgaaagaaagctgaagaaaatgctgggcccctctgctgtagagatagggaatggatgctttgaaaccaaacacaagtgcaaccagacctgtctcgacagaatagctgctggtacctttgatgcaggagaattttctctccccacctttgattcactgaatattactgctgcatctttaaatgacgatggattggataattaccagattttggcgatctattcaactgtcgccagttcattggtactggtagtctccctgggggcaatcagtttctggatgtgctctaatgggtctctacagtgtagaatatgtatttaa

SEQ ID NO: 35 - protein, 574 aa, synthetic construct
MAKNVAIFGLLFSLLVLVPSQIFADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPGGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNYQILAIYSTVASSLVLVVSLGAISFWMCSNGSLQCRICI

SEQ ID NO: 36 - DNA, 1725 bp, synthetic construct
  source 1..1725: mol_type = other DNA
atggcgaaaaacgttgcgattttcggcttattgttttctcttcttgtgttggttccttctcagatcttcgcggatcgaatctgcactgggataacatcgtcaaactcaccacatgtcgtcaaaactgctactcaaggggaggtcaatgtgaccggtgtaataccactgacaacaacacccaccaaatctcattttgcaaatctcaaaggaacagaaaccagggggaaactatgcccaaaatgcctcaactgcacagatctggatgtagccttgggcagaccaaaatgcacagggaaaataccctctgcaagggtttcaatactccatgaagtcagacctgttacatctgggtgctttcctataatgcacgatagaacaaaaattagacagctgcctaaccttctccgaggatacgaacatgtcaggttatcaactcacaacgttatcaataccgaagatgcaccaggaggaccctacgagattggaacctcagggtcttgcctgaacattaccaatggaaagggattcttcgcaacaatggcttgggccgtcccaaaaaacaaaacagcaacaaatccattaacaatagaagtaccatacatttgtaccgaagaggaagaccaaattaccgtttgggggttccactctgacgacgagacccaaatggcaagactctatggggactcaaagccccagaagttcacctcatctgccaacggagtgaccacacattacgtttcacagattggtggcttcccaaatcaaacagaagacggaggactaccacaaagtggcagaattgttgttgattacatggtgcaaaaatctggaaaaacaggaacaattacctatcaaagaggtattttattgcctcaaaaggtgtggtgcgcaagtggcaagagcaaggtaataaaaggatccttgcctttaattggagaagcagattgcctccatgaaaaatacggtggattaaacaaaagcaagccttactacacaggggaacatgcaaaggccataggaaattgcccaatatgggtgaaaacacccttgaagctggccaatggaaccaaatatagacctcctggtggaggatgggaaggaatgattgcaggttggcacggatacacatcccatggggcacatggagtagcggtggcagcagaccttaagagcactcaagaggccataaacaagataacaaaaaatctcaactctttgagtgagatcgaagtaaagaatcttcaaagactaagcggtgccatggatgaactccacaacgaaatactagaactagatgagaaagtggatgatctcagagctgatacaataagctcacaaatagaactcgcagtcctgctttccaatgaaggaataataaacagtgaagatgaacatctcttggcgcttgaaagaaagctgaagaaaatgctgggcccctctgctgtagagatagggaatggatgctttgaaaccaaacacaagtgcaaccagacctgtctcgacagaatagctgctggtacctttgatgcaggagaattttctctccccacctttgattcactgaatattactgctgcatctttaaatgacgatggattggataattaccagattttggcgatctattcaactgtcgccagttcattggtactggtagtctccctgggggcaatcagtttctggatgtgctctaatgggtctctacagtgtagaatatgtatttaa

SEQ ID NO: 37 - protein, 574 aa, synthetic construct
MAKNVAIFGLLFSLLVLVPSQIFADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPGGGWEGMIAGWHGYTSHGAHGVAVAADLKSTQEAINKITKNLNSLSEIEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNYQILAIYSTVASSLVLVVSLGAISFWMCSNGSLQCRICI

SEQ ID NO: 38 - DNA, 6306 bp, synthetic construct
  source 1..6306: mol_type = other DNA
tggcaggatatattgtggtgtaaacaaattgacgcttagacaacttaataacacattgcggacgtttttaatgtactgaattaacgccgaatcccgggctggtatatttatatgttgtcaaataactcaaaaaccataaaagtttaagttagcaagtgtgtacatttttacttgaacaaaaatattcacctactactgttataaatcattattaaacattagagtaaagaaatatggatgataagaacaagagtagtgatattttgacaacaattttgttgcaacatttgagaaaattttgttgttctctcttttcattggtcaaaaacaatagagagagaaaaaggaagagggagaataaaaacataatgtgagtatgagagagaaagttgtacaaaagttgtaccaaaatagttgtacaaatatcattgaggaatttgacaaaagctacacaaataagggttaattgctgtaaataaataaggatgacgcattagagagatgtaccattagagaatttttggcaagtcattaaaaagaaagaataaattatttttaaaattaaaagttgagtcatttgattaaacatgtgattatttaatgaattgatgaaagagttggattaaagttgtattagtaattagaatttggtgtcaaatttaatttgacatttgatcttttcctatatattgccccatagagtcagttaactcatttttatatttcatagatcaaataagagaaataacggtatattaatccctccaaaaaaaaaaaacggtatatttactaaaaaatctaagccacgtaggaggataacaggatccccgtaggaggataacatccaatccaaccaatcacaacaatcctgatgagataacccactttaagcccacgcatctgtggcacatctacattatctaaatcacacattcttccacacatctgagccacacaaaaaccaatccacatctttatcacccattctataaaaaatcacactttgtgagtctacactttgattcccttcaaacacatacaaagagaagagactaattaattaattaatcatcttgagagaaaatggaacgagctatacaaggaaacgacgctagggaacaagctaacagtgaacgttgggatggaggatcaggaggtaccacttctcccttcaaacttcctgacgaaagtccgagttggactgagtggcggctacataacgatgagacgaattcgaatcaagataatccccttggtttcaaggaaagctggggtttcgggaaagttgtatttaagagatatctcagatacgacaggacggaagcttcactgcacagagtccttggatcttggacgggagattcggttaactatgcagcatctcgatttttcggtttcgaccagatcggatgtacctatagtattcggtttcgaggagttagtatcaccgtttctggagggtcgcgaactcttcagcatctctgtgagatggcaattcggtctaagcaagaactgctacagcttgccccaatcgaagtggaaagtaatgtatcaagaggatgccctgaaggtactcaaaccttcgaaaaagaaagcgagtaagttaaaatgcttcttcgtctcctatttataatatggtttgttattgttaattttgttcttgtagaagagcttaattaatcgttgttgttatgaaatactatttgtatgagatgaactggtgtaatgtaattcatttacataagtggagtcagaatcagaatgtttcctccataactaactagacatgaagacctgccgcgtacaattgtcttatatttgaacaactaaaattgaacatcttttgccacaactttataagtggttaatatagctcaaatatatggtcaagttcaatagattaataatggaaatatcagttatcgaaattcattaacaatcaacttaacgttattaactactaattttatatcatcccctttgataaatgatagtacaccaattaggaaggagcatgctcgcctaggagattgtcgtttcccgccttcagtttgcaagctgctctagccgtgtagccaatacgcaaaccgcctctccccgcgcgttgggaattactagcgcgtgtcgacacgcgtggcgcgccctggtatatttatatgttgtcaaataactcaaaaaccataaaagtttaagttagcaagtgtgtacatttttacttgaacaaaaatattcacctactactgttataaatcattattaaacattagagtaaagaaatatggatgataagaacaagagtagtgatattttgacaacaattttgttgcaacatttgagaaaattttgttgttctctcttttcattggtcaaaaacaatagagagagaaaaaggaagagggagaataaaaacataatgtgagtatgagagagaaagttgtacaaaagttgtaccaaaatagttgtacaaatatcattgaggaatttgacaaaagctacacaaataagggttaattgctgtaaataaataaggatgacgcattagagagatgtaccattagagaatttttggcaagtcattaaaaagaaagaataaattatttttaaaattaaaagttgagtcatttgattaaacatgtgattatttaatgaattgatgaaagagttggattaaagttgtattagtaattagaatttggtgtcaaatttaatttgacatttgatcttttcctatatattgccccatagagtcagttaactcatttttatatttcatagatcaaataagagaaataacggtatattaatccctccaaaaaaaaaaaacggtatatttactaaaaaatctaagccacgtaggaggataacaggatccccgtaggaggataacatccaatccaaccaatcacaacaatcctgatgagataacccactttaagcccacgcatctgtggcacatctacattatctaaatcacacattcttccacacatctgagccacacaaaaaccaatccacatctttatcacccattctataaaaaatcacactttgtgagtctacactttgattcccttcaaacacatacaaagagaagagactaattaattaattaatcatcttgagagaaaatgagtcttctaaccgaggtcgaaacgcctatcagaaacgaatgggggtgcagatgcaacgattcaagtgatcctcttgttgttgccgcaagtataattgggattgtgcacctgatattgtggattattgatcgccttttttccaaaagcatttatcgtatctttaaacacggtttaaaaagagggccttctacggaaggagtaccagagtctatgagggaagaatatcgagaggaacagcagaatgctgtggatgctgacgatggtcattttgtcagcatagagctggagtaagagctctaagttaaaatgcttcttcgtctcctatttataatatggtttgttattgttaattttgttcttgtagaagagcttaattaatcgttgttgttatgaaatactatttgtatgagatgaactggtgtaatgtaattcatttacataagtggagtcagaatcagaatgtttcctccataactaactagacatgaagacctgccgcgtacaattgtcttatatttgaacaactaaaattgaacatcttttgccacaactttataagtggttaatatagctcaaatatatggtcaagttcaatagattaataatggaaatatcagttatcgaaattcattaacaatcaacttaacgttattaactactaattttatatcatcccctttgataaatgatagtacaccaattaggaaggaactaggagattgtcgtttcccgccttcagtttgcaagctgctctagccgtgtagccaatacgcaaaccgcctctccccgcgcgttgggaattactagcgcgtgtcgacaagcttgcatgccggtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggtattaaaatcttaataggttttgataaaagcgaacgtggggaaacccgaaccaaaccttcttctaaactctctctcatctctcttaaagcaaacttctctcttgtctttcttgcgtgagcgatcttcaacgttgtcagatcgtgcttcggcaccgcggatggcgaaaaacgttgcgattttcggcttattgttttctcttcttgtgttggttccttctcagatcttcgcctgcaggctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagcgatcgctcaccatcaccatcaccatcaccatcaccattaaaggcctattttctttagtttgaatttactgttattcggtgtgcatttctatgtttggtgagcggttttctgtgctcagagtgtgtttattttatgtaatttaatttctttgtgagctcctgtttagcaggtcgtcccttcagcaaggacacaaaaagattttaattttattatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatctctagagtctcaagcttggcgcgcccacgtgactagtggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgttta

SEQ ID NO: 39 - DNA, 3114 bp, synthetic construct
  source 1..3114: mol_type = other DNA
gtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggtattaaaatcttaataggttttgataaaagcgaacgtggggaaacccgaaccaaaccttcttctaaactctctctcatctctcttaaagcaaacttctctcttgtctttcttgcgtgagcgatcttcaacgttgtcagatcgtgcttcggcaccagtacaatggcgaaaaacgttgcgattttcggcttattgttttctcttcttgtgttggttccttctcagatcttcgcggatcgaatctgcactgggataacatcttcaaactcacctcatgtggtcaaaacagctactcaaggggaggtcaatgtgactggcgtgataccactgacaacaacaccaacaaaatcttattttgcaaatctcaaaggaacaaggaccagagggaaactatgcccggactgtctcaactgtacagatctggatgtggccttgggcaggccaatgtgtgtggggaccacaccttctgctaaagcttcaatactccatgaggtcagacctgttacatccgggtgctttcctataatgcacgacagaacaaaaatcaggcaactacccaatcttctcagaggatatgaaaagatcaggttatcaacccaaaacgttatcgatgcagaaaaagcaccaggaggaccctacagacttggaacctcaggatcttgccctaacgctaccagtaaaatcggattttttgcaacaatggcttgggctgtcccaaaggacaactacaaaaatgcaacgaacccacaaacagtggaagtaccatacatttgtacagaaggggaagaccaaattactgtttgggggttccattcggataacaaaacccaaatgaagagcctctatggagactcaaatcctcaaaagttcacctcatctgctaatggagtaaccacgcattatgtttctcagattggcgacttcccagatcaaacagaagacggaggactaccacaaagcggcagaattgttgttgattacatggtgcaaaaacctgggaaaacaggaacaattgtctatcaaaggggtgttttgttgcctcaaaaggtgtggtgcgcgagtggcaggagcaaagtaataaaagggtcattgcctttaattggtgaagcagattgccttcatgaagaatacggtggattaaacaaaagcaagccttactacacaggaaaacatgcaaaagccataggaaattgcccaatatgggtaaaaacacctttgaagcttgccaatggaaccaaatatagacctcctggtggaggatgggaaggaatgattgcaggttggcacggatacacatctcacggagcacatggagtggcagtggcggcagaccttaagagtacacaagaagctataaataagataacaaaaaatctcaattctttgagtgaactagaagtaaagaaccttcaaagactaagtggtgccatggatgaactccacaacgaaatactcgagctggatgaaaaagtggatgatctcagagctgacactataagctcacaaatagaacttgcagtcttgctttccaacgaaggaataataaacagtgaagacgagcatctattggcacttgagagaaaactaaagaaaatgctgggtccctctgctgtagacataggaaacggatgcttcgaaaccaaacacaaatgcaaccagacctgcttagacaggatagctgctggcacctttaatgcaggagaattttctctccccacttttgattcattgaacattactgctgcatctttaaatgatgatggattggataactaccagattttggcgatctattcaactgtcgccagttcattggtactggtagtctccctgggggcaatcagtttctggatgtgctctaatgggtctctacagtgtagaatatgtatttaaaggcctattttctttagtttgaatttactgttattcggtgtgcatttctatgtttggtgagcggttttctgtgctcagagtgtgtttattttatgtaatttaatttctttgtgagctcctgtttagcaggtcgtcccttcagcaaggacacaaaaagattttaattttattaaaaaaaaaaaaaaaaaagaccgggaattcgatatcaagcttatcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagat

SEQ ID NO: 40 - protein, 223 aa, synthetic construct
  VAR_SEQ 78: note = L or I
  VAR_SEQ 129: note = S or absent
  VAR_SEQ 132: note = E or D
  VAR_SEQ 158: note = N or D
  VAR_SEQ 159: note = A or D
  VAR_SEQ 204: note = L or I
  VAR_SEQ 208: note = I or V
GFFGAIAGFLEGGWEGMIAGWHGYTSHGAHGVAVAADLKSTQEAINKITKNLNSLSELEVKNLQRLSGAMDELHNEIXELDEKVDDLRADTISSQIELAVLLSNEGIINSEDEHLLALERKLKKMLGPXAVXIGNGCFETKHKCNQTCLDRIAAGTFXXGEFSLPTFDSLNITAASLNDDGLDNHTILLYYSTAASSLAVTLMXAIFXVYMVSRDNVSCSICL

SEQ ID NO: 41 - protein, 187 aa, synthetic construct
  VAR_SEQ 58: note = I
  VAR_SEQ 78: note = L or I
  VAR_SEQ 129: note = S or absent
  VAR_SEQ 132: note = E or D
  VAR_SEQ 158: note = N or D
  VAR_SEQ 159: note = A or D
GFFGAIAGFLEGGWEGMIAGWHGYTSHGAHGVAVAADLKSTQEAINKITKNLNSLSEXEVKNLQRLSGAMDELHNEIXELDEKVDDLRADTISSQIELAVLLSNEGIINSEDEHLLALERKLKKMLGPXAVXIGNGCFETKHKCNQTCLDRIAAGTFXXGEFSLPTFDSLNITAASLNDDGLDNHTI

SEQ ID NO: 42 - protein, 164 aa, synthetic construct
  VARIANT 35: note = I
  VARIANT 55: note = L or I
  VARIANT 106: note = S or absent
  VARIANT 109: note = E or D
  VARIANT 135: note = N or D
  VARIANT 136: note = A or D
YTSHGAHGVAVAADLKSTQEAINKITKNLNSLSEXEVKNLQRLSGAMDELHNEIXELDEKVDDLRADTISSQIELAVLLSNEGIINSEDEHLLALERKLKKMLGPXAVXIGNGCFETKHKCNQTCLDRIAAGTFXXGEFSLPTFDSLNITAASLNDDGLDNHTI

SEQ ID NO: 43 - protein, 552 aa, Influenza virus 5 A/Indonesia/5/2005
DQICIGYHANNSTEQVDTIMEKNVTVTHAQDILEKTHNGKLCDLDGVKPLILRDCSVAGWLLGNPMCDEFINVPEWSYIVEKANPTNDLCYPGSFNDYEELKHLLSRINHFEKIQIIPKSSWSDHEASSGVSSACPYLGSPSFFRNVVWLIKKNSTYPTIKKSYNNTNQEDLLVLWGIHHPNDAAEQTRLYQNPTTYISIGTSTLNQRLVPKIATRSKVNGQSGRMEFFWTILKPNDAINFESNGNFIAPEYAYKIVKKGDSAIMKSELEYGNCNTKCQTPMGAINSSMPFHNIHPLTIGECPKYVKSNRLVLATGLRNSPQRESRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESIRNGTYNYPQYSEEARLKREEISGVKLESIGTYQILSIYSTVASSLALAIMMAGLSLWMCSNGSLQCRICI

(19) United States
(12) Patent Application Publication
(10) Pub. No.: US 2026/0317378 A1
(43) Pub. Date: Oct. 15, 2026

(54) MODIFIED INFLUENZA B VIRUS HEMAGGLUTININ

(71) Applicant: ARAMIS BIOTECHNOLOGIES INC., Quebec (CA)

(72) Inventors: Aurelien LORIN; Pierre-Olivier LAVOIE, Quebec (CA)

(21) Appl. No.: 19/106,616

(22) PCT Filed: Aug. 25, 2022
(86) PCT No.: PCT/CA2022/051285

§ 371 Date: Feb. 25, 2025

(51) Int. Cl. C07K 14/005; A61K 39/00; A61K 39/145; C12N 7/00

(52) U.S. Cl. CPC C07K 14/005; A61K 39/145; C12N 7/00; A61K 2039/5258; A61K 2039/70; C12N 2760/16123; C12N 2760/16134; C12N 2760/16222; C12N 2760/16223; C12N 2760/16234

(57) ABSTRACT

The present disclosure relates to the production of modified influenza B hemagglutinin (HA) protein. More specifically, the present disclosure relates to producing and increasing influenza virus-like particle (VLP) production in plants, wherein the VLPs comprise the modified influenza B HA proteins. The HA protein may comprise an amino acid sequence comprising at least one substitution when compared to a corresponding wildtype amino acid sequence. Further provided are nucleic acid encoding the modified B HA protein. Furthermore methods of producing an influenza virus like particle (VLP) and methods of increasing yield of production of an influenza virus like particle (VLP) in a host or host cell are also provided.

### FIELD OF INVENTION

The present disclosure relates to modified influenza B virus hemagglutinin (HA) proteins and virus-like particle comprising modified influenza B hemagglutinin (HA) proteins. The disclosure further relates to the production of modified influenza B hemagglutinin (HA) proteins and virus-like particle in a host or host cell.

### BACKGROUND OF THE INVENTION

Influenza viruses are enveloped, single-stranded-RNA viruses of the Orthomyxoviridae family. Influenza viruses are highly contagious and can cause mild to serious illness across all age groups.

Influenza viruses are highly pleomorphic particles composed of two surface glycoproteins, hemagglutinin (HA) and neuraminidase (NA). The HA mediates attachment of the virus to the host cell and viral-cell membrane fusion during penetration of the virus into the cell. The influenza virus genome consists of eight single-stranded negative-sense RNA segments of which the fourth largest segment encodes the HA gene.

The HA molecule is present in the virion as a trimer. Each monomer exists as two chains, HA1 and HA2 domains (also referred to as HA1 and HA2 subunits or subdomains), linked by a single disulfide bond. Infected host cells produce a precursor glycosylated polypeptide (HA0) with a molecular weight of about 85 kDa which is subsequently cleaved into HA1 (~40 kDa) and HA2 (~20 kDa) domains. After cleavage, the two disulfide-bonded protein domains adopt the requisite conformation necessary for viral infectivity.

The membrane distal globular head constitutes the majority of the HA1 structure and contains the sialic acid binding pocket for viral entry and major antigenic domains. HA1 contains vestigial esterase domains E1′ and E2 and a receptor-binding site (RBS) with the RBS being the least conserved segment of the influenza virus. HA2 is a single-pass integral membrane protein with fusion peptide (FP), soluble HA2 ectodomain, transmembrane (TM), and cytoplasmic tail (CT) (see FIG. 1). HA2 together with the N and C terminal HA1 residues forms a stalk domain, which includes the transmembrane region, and is relatively conserved. The stalk structure contains the fusion machinery, which undergoes a conformational change in the low pH environment of late endosomes to trigger membrane fusion and penetration into cells. The relative conservation of the stalk domain might be due to its immunosubdominant nature (and hence the lack of antibody pressure), but it is also likely that this observation is caused by a lack of tolerance to changes due to the functional constraints of the fusion machinery. (Kirkpatrick et al. Scientific Reports volume 8, Article number: 10432 (2018)).

The influenza viruses are divided into types A, B and C based on antigenic differences. Influenza A and B are the causative organism for seasonal disease epidemics in humans. In contrast to influenza A, a zoonotic pathogen that infects multiple host species, influenza B primarily infects humans and, rarely, seals. Unlike influenza A, there is limited antigenic drift observed in influenza B virus, making the virus relatively stable. Accordingly, the sequence identity between influenza A and B virus HA is low approximately 20% for HA1, which is the primary target for antigenic variation.

Though its lack of antigenic diversity bars pandemic outbreaks, influenza B contributes to seasonal occurrences of influenza, which can result in serious infections costing thousands of lives and billions of dollars. Influenza B has been of increasing concern lately, due to the rise in circulation of two distinct lineages of the virus: Victoria lineage and Yamagata lineage.

Various mutations in influenza HA proteins, particularly in HA protein from influenza A, have been investigated.

For example, Castelán-Vega et al. (Adv Appl Bioinform Chem. 2014; 7:37-44) used a stability prediction algorithm to compare 7,479 full-length amino acid sequences of HA from the influenza A (H1N1)pdm09 virus and identified that D104N, A259T, S124N, and E172K mutations resulted in a predicted enhancement of influenza HA stability. In contrast, S206T, K285E, and E47K mutations had a predicted destabilizing effect on HA.

Reed et al. 2010 (J. Virol. 83:3568-3580) generated four recombinant H5N1 viruses containing mutations that altered the acid stability of the HA protein without changing its level of expression, cleavage, receptor binding, or membrane fusion efficiency. Two of the mutations increased the pH of membrane fusion of the H5N1 HA protein (Y23₁H and N114₂K), and the other two mutations reduced the pH of fusion (H24₁Q and K58₂I).

Zaraket et al. 2013 (J Virol. 2013 May; 87 (9)) investigated how mutations that alter the activation pH of the HA protein influence the fitness of an avian H5N1 influenza virus in a mammalian model. Zaraket et al. 2013 (J Virol. 2013 September; 87 (17)) investigated how a decrease in the HA activation pH (an increase in acid stability) influences the properties of highly pathogenic H5N1 influenza virus in mammalian hosts.

Though less numerous, mutations in HA protein from influenza B, have also been investigated.

For example, Lugovtsev et al. 2007 (Virology, 2007 September; 365 (2)) used reverse genetics, to analyze the contribution to virus growth of amino acid substitutions that had previously been identified in a high growth virus phenotype of B/Victoria/504/2000. They found that G141E and R162M were most favorable for virus growth; however, only R162M could improve virus growth without antigenic alteration.

Chen et al. 2007 (Vaccine, 2007 January; 26 (13)) studied the effect of the 196/197 glycosylation site on influenza B virus growth and antigenicity.

Kim et al. 2015 (Vaccine, 2015 September; 33) investigate mutations responsible for improved growth in cold-adapted influenza B viruses. Molecular analysis revealed that the following mutations in the HA, NP and NA genes are required for enhanced viral growth: G156/N160 in the HA, E253, G375 in the NP and T146 in the NA genes.

Vaccination remains the most effective method to prevent influenza infection. However, the constantly evolving nature of influenza viruses requires continuous global monitoring and frequent reformulation of influenza vaccines. The World Health Organization (WHO) convenes technical consultations in February and September each year to recommend viruses for inclusion in seasonal influenza vaccines for the northern and southern hemispheres, respectively. These recommendations are based on information provided by the WHO Global Influenza Surveillance and Response System (GISRS).

Quadrivalent influenza vaccines (QIV) vaccines contain hemagglutinin antigen (HA) for each of the four influenza strains recommended by the WHO for upcoming influenza season (usually an influenza A (H3N2) and A (H1N1) strain and two influenza B strains, one from each B virus lineage [B/Yamagata and B/Victoria]).

Conventionally, vaccination is accomplished using live attenuated or whole inactivated forms of the virus, which elicit an immune response when administered to a patient. To eliminate the potential risk of live attenuated and whole inactivated viruses re-acquiring the competency to replicate and become infectious, vaccines comprising recombinant viral proteins have also been used to elicit protective immunity to influenza infection.

However, the use of recombinant viral proteins as the immunogenic component of vaccines is subject to a number of limitations. Firstly, in the absence of the full complement of viral proteins and genetic components required for optimal expression and proper protein folding, the yield of recombinant viral proteins in standard in vitro expression systems may be insufficient for the purpose of vaccine production. Second, recombinant viral protein vaccines may exhibit poor immunogenicity, owing to improper folding, poor antigen presentation, and/or the generation of a primarily humoral immune response that is ineffective in conferring long-lasting, protective immunity.

Virus-like particles (VLPs) are potential candidates for inclusion in immunogenic compositions. VLPs closely resemble mature virions, but they do not contain viral genomic material. Therefore, VLPs are non-replicative in nature, which make them safe for administration as a vaccine. In addition, VLPs can be engineered to express viral glycoproteins on the surface of the VLP, which is their most native physiological configuration. Moreover, since VLPs resemble intact virions and are multivalent particulate structures, VLPs may be more effective in inducing neutralizing antibodies to the glycoprotein than soluble envelope protein antigens.

VLPs have been produced in plants before (see for example WO2009/076778; WO2009/009876; WO 2009/076778; WO 2010/003225; WO 2010/003235; WO2010/006452; WO2011/03522; WO 2010/148511; WO 2013/044390 and WO2014153674, which are incorporated herein by reference).

WO2009/076778 teaches a method of producing influenza VLPs in plants comprising the step of introducing a nucleic acid having a regulatory region active in the plant operatively linked to a nucleotide sequence encoding an influenza HA from a type A or type B influenza.

WO2009/009876 teaches a method of producing influenza HA VLPs in plants, wherein influenza HA self-assembles into VLPs in plant cells and bud from plant cell membranes.

WO2010/006452 teaches the production of VLPs comprising modified influenza HA proteins, wherein glycosylation sites at positions 154, 165, 286, or combinations thereof (with reference to A/Vietnam/1194/04 [H5N1] numbering), have been abolished by mutating the residues at said positions to amino acids other than asparagine. WO2010/006452 further teaches that amino acids at positions 156, 167, 288, or combinations thereof, may be mutated to residues other than serine or threonine to similarly abolish the N-linked glycosylation signal triad “N-X-S/T”. By selectively deleting glycosylation sites located in the globular head of the HA protein, WO2010/006452 demonstrates that the resulting HA protein has increased antigenicity and broader cross-reactivity.

WO2010/148511 discloses a method for producing influenza VLPs in plants, wherein the VLPs comprise chimeric HA proteins. The chimeric HA proteins comprise a stem domain cluster having an F′1, F′2 and F subdomain; a head domain cluster having an RB, E1 and E2 subdomain; and a transmembrane domain cluster having a transmembrane domain and a C-terminal tail domain, wherein at least one subdomain is derived from a first influenza strain and the other subdomains are derived from one or more second influenza strain.

WO2014/153674 teaches a method of producing influenza VLPs in a plant, wherein the VLPs comprise modified influenza HA having a modified proteolytic loop. The modified proteolytic loop comprises the removal of the proteolytic cleavage site between HA1 and HA2 domains of the HA0 precursor. The HA protein is thus stabilized and increased protein yields are achieved as compared to native HA protein.

WO 2013/044390 teaches the production of virus like particle (VLP) by co-expressing influenza HA and a proton channel protein in a plant.

### SUMMARY OF THE INVENTION

The present invention relates to the production of modified influenza B hemagglutinin (HA) protein. The invention is also directed to virus like particle (VLP) that comprise the modified influenza B HA protein. The modified B HA protein and the VLP comprising the modified B HA show improved characteristics when compared to unmodified B HA or VLPs that comprise the unmodified B HA protein. The present invention further relates to producing and increasing influenza virus-like particle (VLP) production in a host or host cell, wherein the VLPs comprise the modified influenza B HA protein.

It is an object of the invention to provide an improved method to increase influenza VLP production in a host or host cell, such for example in a plant or plant cell.

According to the present invention, there is provided a modified influenza B virus hemagglutinin (HA) protein comprising a modified HA2 ectodomain, wherein the modified HA2 ectodomain comprises an amino acid sequence with at least one amino acid substitution compared to a parent HA2 ectodomain amino acid sequence, wherein the at least one substitution corresponds to amino acid position 402 in sequence alignment with reference sequence of SEQ ID NO: 1 (B/Washington/09/19 HA). The parent HA2 ectodomain amino acid sequence may be a wildtype amino acid sequence of an influenza B virus.

The substitution may be a substitution to a non-leucine. For example, the substitution may be to an isoleucine or a conserved substitution of isoleucine. The conserved substitution of isoleucine may be methionine, phenylalanine or valine.

The modified B HA may comprise a modified HA2 subunit, wherein the modified HA2 subunit may comprise a sequence that may have from 80% to 100% identity with the sequence of SEQ ID NO: 41 or SEQ ID NO:42. Furthermore, the modified HA2 subunit may comprise a modified HA2 ectodomain. The modified HA2 ectodomain may comprise a sequence that may have from 80% to 100% identity with the sequence of SEQ ID SEQ ID NO:42. The sequence of the influenza B HA protein may comprise from 80% to 100% identity with the sequence of SEQ ID NO: 13, 17, 21, 25, 29, 33 or 37.

The modified influenza B HA may comprise plant-specific N-glycans, modified N-glycans or a combination thereof. Furthermore, the modified influenza B HA protein may be a chimeric B HA protein, wherein the chimeric B HA protein comprises a transmembrane and cytoplasmic tail (TM/CT) derived from an influenza A HA protein. In addition, the modified influenza B HA protein may have a modified proteolytic cleavage site. Therefore, it is also provided a modified influenza B HA protein, wherein the proteolytic cleavage site has been modified.

It is further provided a nucleic acid comprising a nucleotide sequence encoding the modified influenza HA protein as described above.

It is also provided a virus-like particle (VLP) comprising the modified influenza B HA protein as described above.

In another aspect it is provided a method (A) of producing a modified influenza B HA protein in a non-human host or host cell comprising:
a) introducing the nucleic acid comprising a nucleotide sequence encoding the modified influenza HA protein as described above into the non-human host or host cell, or providing the non-human host or host cell comprising the nucleic acid comprising a nucleotide sequence encoding the modified influenza HA protein as described above, and
b) incubating the non-human host or host cell under conditions that permit the expression of the nucleic acid, thereby producing the modified influenza B HA protein.

In another aspect it is provided a method (B) of increasing yield of an influenza B HA protein in a non-human host or host cell, comprising:
a) introducing the nucleic acid comprising a nucleotide sequence encoding the modified influenza HA protein as described above into the non-human host or host cell; or providing the non-human host or host cell comprising the nucleic acid comprising a nucleotide sequence encoding the modified influenza HA protein as described above; and
b) incubating the non-human host or host cell under conditions that permit expression of the modified B HA protein encoded by the nucleic acid, thereby producing the modified B HA at a higher yield compared to non-human host or host cell expressing an influenza B HA protein, comprising the HA2 ectodomain parent amino acid sequence.

The modified influenza B HA protein in method (A) or (B) may further be extracted and purified from the non-human host or host cell.

In another aspect it is provided a modified influenza B HA protein produced by method (A) or method (B).

In another aspect it is provided a method (C) producing an influenza virus like particle (VLP) in a non-human host or host cell, comprising:
a) providing the non-human host or host cell comprising the nucleic acid comprising a nucleotide sequence encoding the modified influenza HA protein; or introducing into the non-human host or host cell the nucleic acid comprising a nucleotide sequence encoding the modified influenza HA protein; and
b) incubating the non-human host or host cell under conditions that permit expression of the modified influenza B HA protein encoded by the nucleic acid, thereby producing the VLP.

In yet another aspect it is provided a method (D) of increasing yield of an influenza virus like particle (VLP) in a non-human host or host cell, comprising:
a) introducing the nucleic acid comprising a nucleotide sequence encoding the modified influenza HA protein into the non-human host or host cell; or providing the non-human host or host cell comprising the nucleic acid comprising a nucleotide sequence encoding the modified influenza HA protein; and
b) incubating the non-human host or host cell under conditions that permit expression of the modified B HA protein encoded by the nucleic acid, thereby producing the VLP at a higher yield compared to non-human host or host cell expressing an influenza B HA protein comprising the HA2 ectodomain parent amino acid sequence.

The method of (C) or (D) may further comprise step c), harvesting the non-human host or host cell, and extracting and purifying the VLP.

It is further provided a virus-like particle (VLP) produced by the method of (C) or (D). The VLP may further comprise one or more than one lipid derived from the non-human host or host cell.

The nucleic acid in the method of (A), (B), (C) or (D) may further comprises a nucleotide sequence encoding a proton channel protein. Or step a) of the method of (A), (B), (C) or (D) may further comprise introducing a second nucleic acid encoding a proton channel protein; and step b) of the method of (A), (B), (C) or (D) further comprises incubating the non-human host or host cell under conditions that permit expression of the proton channel protein encoded by the second nucleic acid. The proton channel protein may be influenza A M2 protein.

In another aspect it is provided a method of producing an antibody or antibody fragment, the method comprising, administering the VLP as described to a subject, or a host animal, thereby producing the antibody or the antibody fragment. It is further provided an antibody produced by the above method.

In another aspect it is provided a host or host cell comprising the nucleic acid, the modified influenza B HA protein, the VLP, or a combination thereof.

In a further aspect it is provided, a composition for inducing an immune response comprising, an effective dose of the VLP, and a pharmaceutically acceptable carrier, adjuvant, vehicle or excipient.

It is also provided a vaccine for inducing an immune response, the vaccine comprising an effective dose of the modified influenza B HA protein, the VLP, or the composition as described above. The vaccine may further comprise an adjuvant.

It is further provided a method for inducing an immune response a subject, the method comprising administering the VLP, the composition or the vaccine to the subject. The VLP, composition or vaccine may be administered to the subject orally, intranasally, intramuscularly, intraperitoneally, intravenously or subcutaneously.

The non-human host or host cell may comprise a plant, portion of a plant, a plant cell, a fungi, a fungi cell, an insect, an insect cell, an animal or an animal cell.

In another aspect it is also provided a multivalent immunogenic composition comprising two or more than two types of VLP, wherein at least one type of VLP comprises the modified influenza B HA as described above. The composition may further comprise a second type of VLP, wherein the second type of VLP comprises the modified influenza B HA. The at least one type of VLP may be a first type VLP and wherein the first type VLP may comprise modified B HA that are derived from a different influenza B lineage than the modified B HA of the second type of VLP. For example the first type VLP may comprise modified B HA derived from B/Victoria lineage and the modified B HA in the second type VLP may be derived from the B/Yamagata lineage. The composition may further comprise one or more than one type of VLP comprising influenza A HA protein. For example the influenza A HA may be derived from influenza subtype H1 and/or from influenza subtype H3.

In yet another aspect it is provide a quadrivalent immunogenic composition comprising a first type of VLP comprising the modified influenza B HA as described herewith, a second type of VLP comprising the modified influenza B HA as described herewith, a third type of VLP comprising influenza A HA and a fourth type of VLP comprising influenza A HA, wherein the first type VLP comprises modified B HA that are derived from a different influenza B lineage than the modified B HA of the second type of VLP.

This summary of the invention does not necessarily describe all features of the invention.

### BRIEF DESCRIPTION OF THE DRAWINGS

These and other features of the invention will become more apparent from the following description in which reference is made to the appended drawings wherein:

FIG. 1 shows a schematic representation of domain structure of the HA protein (precursor HA0). HA0 contains an N-terminal signal sequence (which targets protein synthesis to the ER before being cleaved and released) and two HA subunits (HA1 and HA2). Domains in the HA2 subunit include the Fusion protein (FP), HA2 ectodomain, transmembrane domain (TM) and the cytoplasmic tail (CT). The HA0 precursor protein is incapable of causing membrane fusion, and proteolytic cleavage of the HA1 and HA2 subunit is required to prime the protein into a fusion-competent form. The cleavage site between the HA1 and HA2 subunit is indicated by an arrow.

FIG. 2A shows a schematic representation of vector 4498 used for the assembly of the vector plasmids encoding modified Influenza strain B HA protein.

FIG. 2B shows a schematic representation of vector 2879 encoding the Influenza B strain HA from B/Singapore/INFKK-16-0569/2016. FIG. 2C shows a schematic representation of vector 8894 encoding the modified Influenza B strain HA from B/Singapore/INFKK-16-0569/2016 with a L404I mutation. FIG. 2D shows a schematic representation of vector 7679 encoding the Influenza B strain HA from B/Washington/02/2019. FIG. 2E shows a schematic representation of vector 8881 encoding the Influenza B strain HA from B/Washington/02/2019 with a L402I mutation. FIG. 2F shows a schematic representation of vector 8424 encoding the Influenza B strain HA from B/Rhode Island/01/2019. FIG. 2G shows a schematic representation of vector 7787 encoding the Influenza B strain HA from B/Rhode Island/01/2019 with a L402I mutation. FIG. 2H shows a schematic representation of vector 9627 encoding the Influenza B strain HA from B/Michigan/01/2021. FIG. 2I shows a schematic representation of vector 9628 encoding the Influenza B strain HA from B/Michigan/01/2021 with a L402I mutation. FIG. 2J shows a schematic representation of vector 9629 encoding the Influenza B strain HA from B/Henan-Xigong/1118/2021. FIG. 2K shows a schematic representation of vector 9630 encoding the Influenza B strain HA from B/Henan-Xigong/1118/2021 with a L402I mutation. FIG. 2L shows a schematic representation of vector 9866 encoding the Influenza B strain HA from B/Singapore/WUH4618/2021. FIG. 2M shows a schematic representation of vector 9867 encoding the Influenza B strain HA from B/Singapore/WUH4618/2021 with a L402I mutation. FIG. 2N shows a schematic representation of vector 9868 encoding the Influenza B strain HA from B/Austria/1359417/2021. FIG. 2O shows a schematic representation of vector 9869 encoding the Influenza B strain HA from B/Austria/1359417/2021 with a L402I mutation.

FIG. 3 shows in planta yield fold-change, expressing modified influenza B HA proteins, with fold-change calculated in relation to the appropriate unmodified (parent) control HA protein (CTL), as follows: B/Singapore/INFKK-16-0569/2016 (CTL: Construct 2879, L404I: Construct 8894), B/Washington/02/2019 (CTL: Construct 7679, L402I: Construct 8881), B/Rhode Island/01/2019 (CTL: Construct 8424, L402I: Construct 7787), B/Michigan/01/2021 (CTL: Construct 9627, L402I: Construct 9628), B/Henan-Xigong/1118/2021 (CTL: Construct 9629, L402I: Construct 9630), B/Singagore/WUH4618/2021 (CTL: Construct 9866, L402I: Construct 9867), and B/Austria/1359417/2021 (CTL: Construct 9868, L402I: Construct 9869).

FIG. 4 shows yield fold change of a drug substance (DS) obtained from either a host expressing modified influenza B strain HA proteins, with fold-change calculated in relation to the appropriate parent control HA protein (CTL), as follows: B/Washington/02/2019 (CTL: Construct 7679, L402I: Construct 8881), and B/Rhode Island/01/2019 (CTL: Construct 8424, L402I: Construct 7787).

### DETAILED DESCRIPTION

The following description is of a preferred embodiment.

As used herein, the terms “comprising”, “having”, “including”, “containing”, and grammatical variations thereof, are inclusive or open-ended and do not exclude additional, un-recited elements and/or method steps. The term “consisting essentially of” when used herein in connection with a product, use or method, denotes that additional elements and/or method steps may be present, but that these additions do not materially affect the manner in which the recited method or use functions. The term “consisting of” when used herein in connection with a product, use or method, excludes the presence of additional elements and/or method steps. A product, use or method described herein as comprising certain elements and/or steps may also, in certain embodiments, consist essentially of those elements and/or steps, and in other embodiments consist of those elements and/or steps, whether or not these embodiments are specifically referred to. In addition, the use of the singular includes the plural, and “or” means “and/or” unless otherwise stated. Unless otherwise defined herein, all technical and scientific terms used herein have the same meaning as commonly understood by one of ordinary skill in the art. As used herein, the term “about” refers to an approximately +/−10% variation from a given value. It is to be understood that such a variation is always included in any given value provided herein, whether or not it is specifically referred to. The use of the word “a” or “an” when used herein in conjunction with the term “comprising” may mean “one,” but it is also consistent with the meaning of “one or more,” “at least one” and “one or more than one.”

Modified influenza B hemagglutinin (HA) proteins (also termed modified B HA protein, modified influenza B HA protein, modified B HA, modified influenza B HA, mutant B HA, influenza mutant B HA, modified B protein; modified B; influenza or influenza B HA comprising a modified HA2 subunit or a modified HA2 ectodomain) and methods of producing modified influenza B HA proteins in a host or host cell are described herein. Furthermore, the modified influenza B HA protein may self-assemble into virus-like particle (VLP). Therefore, it is also provided influenza VLP that comprise or consist of modified influenza B HA protein.

It has been observed that the modification for example by substitution of specific amino acids in B HA proteins for example B HA from the Yamagata lineage or Victoria lineage results in improved characteristics of the modified B HA protein when compared to a parent HA which does not include the specific amino acid substitution. The parent HA may also be referred to as unmodified B HA protein. In some embodiments, the parent or unmodified HA may be a wild type HA. In other embodiments, the parent or unmodified HA may comprise other modifications, such as, for example, deletion or partial deletion of the proteolytic loop and/or replacement of the native transmembrane and cytoplasmic tail domain (TMCT) with the TMCT from an influenza A HA, as described below.

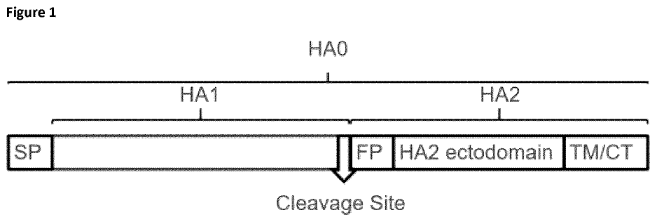

With reference to influenza virus, the term “hemagglutinin” or “HA” as used herein refers to a glycoprotein found on the outside of influenza viral particles. HA is translated as a single protein, HA0. HA0 generally comprises a signal peptide (SP), an HA1 domain (also referred to as HA1 subunit), an HA2 domain (also referred to as HA2 subunit) comprising the fusion protein (FP), the HA2 ectodomain and the transmembrane domain (TM) and the cytoplasmic tail (CT), collectively referred to as TM/CT (see FIG. 1).

For viral activation, HA0 (assembled as trimers) must be cleaved at a specific site between the HA1 and HA2 domains of the protein. After cleavage, the two disulfide-bonded protein domains produce the mature form of the protein subunits as a prerequisite for the conformational change necessary for fusion and hence viral infectivity.

Nucleotide sequences encoding HA, as well as HA amino acid sequences are well known and are available-see, for example, the BioDefence Public Health base (Influenza Virus; see URL: biohealthbase.org) or National Center for Biotechnology Information (see URL: ncbi.nlm.nih.gov), both of which are incorporated herein by reference. Furthermore, influenza strains may be identified and classified by techniques know within the art for example by hemagglutination inhibition assay, reverse transcriptase PCR, real-time PCR or sequencing (ElHefnawi & Sherif (Virology, Volume 449, 20 Jan. 2014).

The modified B HA protein may comprise an HA1 domain, an HA2 ectodomain, a transmembrane domain (TM) and a cytoplasmic tail (CT). The HA1 domain and HA2 domain may be derived from an influenza B HA and the transmembrane domain (TM) and the cytoplasmic tail (CT) may be derived from an influenza A HA. The modified HA protein may further comprise cleavage site and a fusion peptide. In some embodiments the cleavage site and/or fusion peptide may be modified. As further described below the modified B HA may be produced as a precursor protein and may comprise a native or non-native signal peptide.

The modified influenza B HA proteins disclosed herewith comprise modifications or mutations that have been found to result in improved B HA characteristics as compared to the parent (unmodified) HA proteins of the same strain or subtype of influenza that does not comprise the modification(s) or mutation(s), referred to as parent HA, unmodified HA or control. For example, the modified influenza B HA protein may have an amino acid sequence with at least one substitution of an amino acid when compared to a corresponding parent amino acid sequence. In some embodiments, the modified B HA protein may have one or more than one substitution in the HA2 ectodomain when compared to the sequence of the HA2 ectodomain of a parent B HA.

For example the modified influenza B virus hemagglutinin (HA) protein may comprise a modified HA2 subunit (also referred to as HA2), wherein at least one amino acid is modified (for example by substitution or replacement) compared to parent sequence, such for example a wild type amino acid in the sequence. In ones aspect the modified influenza B virus hemagglutinin (HA) protein may comprise a modified HA2 ectodomain, wherein at least one amino acid is modified (for example by substitution or replacement) compared to parent sequence, such for example a wild type amino acid in the sequence. The amino acid modification may correspond to amino acid position 402 in sequence alignment with reference sequence of SEQ ID NO: 1 (B/Washington/09/19 HA).

Examples of improved characteristics of the modified B HA protein include, increased HA B protein yield when expressed in a host or host cell as compared to the parent B HA protein of the same strain of influenza that does not comprise the modification or mutation; increased VLP yield when modified B HA protein is expressed in host or host cell as compared to the level of VLP production, wherein the B HA protein does not comprise the modification or mutation; increased Drug Substance (DS) yield, when the DS is obtained from host or host cell that express the modified HA B protein compared to DS yield obtained from host or host cell that express parent (unmodified) HA B protein and a combination thereof.

The modified B virus HA protein may be created by introducing changes to the amino acid sequence of influenza B HA protein that results in an improved characteristic of the HA as described above. Isolation of nucleic acids encoding such HA molecules is routine, as is modification of the nucleic acid to introduce changes in the amino acid sequence, e.g., by site-directed mutagenesis.

The influenza B HA protein, mutant B HA protein or modified B HA protein as described herein is modified and comprises one or more than one mutation, modification, or substitution in its amino acid sequence, wherein at least an amino acid that corresponds with amino acid at position 402 of B/Washington/09/19 HA (SEQ ID NO: 1) or that corresponds with amino acid at position 404 of B/Singapore/INFKK-16-0569/2016 (SEQ ID NO: 2) has been modified, when compared to the unmodified (parent) sequence.

By “correspond to an amino acid” or “corresponding to an amino acid”, it is meant that an amino acid corresponds to an amino acid in a sequence alignment with an influenza reference strain as described herein.

The amino acid residue number or residue position of HA is in accordance with the numbering of the HA of an influenza reference strain. For example, the reference strain may be B/Washington/09/19 HA (SEQ ID NO: 1), which belongs to the Victoria lineage (see Table 1). The reference strain may also be B/Singapore/INFKK-16-0569/2016 (SEQ ID NO: 2), which belongs to the Yamagata lineage (see Table 1).

The corresponding amino acid positions may be determined by aligning the sequence of the B HA with the sequence of HA of their respective reference strain. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Natl. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see, e.g., Current Protocols in Molecular Biology (Ausubel et al., eds. 1995 supplement)).

When referring to modifications, mutants or variants, the wildtype amino acid residue (also referred to as simply ‘amino acid’) is followed by the residue number and the new or substituted amino acid. For example, substitution of Leucine (L) for Isoleucine (I) in residue or amino acid at position 402 is denominated L402I.

The modified B HA, B HA mutant or variant is designated in the same manner by using the single letter amino acid code for the unmodified (parent) or wildtype residue followed by its position and the single letter amino acid code of the replacement residue.

TABLE 1
Corresponding positions of modification
in HA of different influenza B strains.

| | | Position | SEQ |
|---|---|---|---|
| Influenza Strain | Lineage | in HA* | ID NO:** |
| B/Washington/02/2019 | Victoria | 402 | 1 |
| B/Singapore/WUH4618/2021 | Victoria | 402 | 7 |
| B/Rhode Island/01/2019 | Victoria | 402 | 3 |
| B/Michigan/01/2021 | Victoria | 402 | 4 |
| B/Henan-Xigong/1118/2021 | Victoria | 402 | 5 |
| B/Austria/1359417/2021 | Victoria | 402 | 6 |
| B/Singapore/INFKK-16-0569/2016 | Yamagata | 404 | 2 |

*HA0 (without signal peptide)
**wild type sequence

By “modification”, “amino acid modification”, or “amino acid sequence modification” it is meant a mutation, substitution, replacement or deletion of one or more than one amino acid residues in a sequence compared to the original parent (unmodified) sequence. The parent sequence may be a wild type sequence or the parent sequence may be a sequence that already comprises modifications (“parent modifications”) when compared to a wild type sequence. By “amino acid substitution” or “substitution” it is meant the replacement of an amino acid in the amino acid sequence of a protein with a different amino acid compared to the parent sequence. In one embodiment, the modified influenza B virus hemagglutinin (HA) protein comprises a substitution in the HA2 ectodomain compared to a parent HA2 ectodomain.

The terms amino acid, amino acid residue or residue are used interchangeably in the disclosure. One or more than one amino acid may be replaced with one or more amino acids that are different than the original amino acid at this position, without changing the overall length of the amino acid sequence of the protein. The substitution or replacement may be experimentally induced by altering the codon sequence in a nucleotide sequence encoding the protein to the codon sequence of a different amino acid compared to the original amino acid of the parent sequence. Furthermore, one or more than one amino acid may be deleted from the amino acid sequence of the protein. The resulting protein is a modified influenza B HA protein. The modified B HA protein does not occur naturally.

The modified B HA includes non-naturally occurring HA protein, having at least one modification to parent HA or naturally occurring HA and having improved characteristics compared to the parent HA or naturally occurring HA protein from which the amino acid sequence of the modified B HA is derived. Modified B HA proteins have an amino acid sequence, not found in nature, which is derived by replacement of one or more amino acid residues of an HA protein with one or more different amino acids.

Accordingly, modified B HA, mutant B HA or recombinant B HA refers to an HA in which the DNA sequence encoding the parent HA is modified to produce a modified or mutant DNA sequence which encodes the modification, mutation or substitution of one or more amino acids in the HA amino acid sequence.

The modified influenza B HA protein or mutant influenza B HA protein as described herein is modified and comprises a mutation, or modification, a residue in sequence alignment with positions 402 of B/Washington/02/2019 (SEQ ID NO: 1). It is therefore provided influenza B HA polypeptides, proteins, and/or protein complexes such as for example virus-like particle (VLP) that comprise modifications or mutations at amino acid position 402, where such amino acid numbering is based upon the sequence of B/Washington/02/2019 (SEQ ID NO: 1), or at amino acid positions that correspond to such amino acid positions, for example as determined by alignment of an B HA amino acid sequence to SEQ ID NO: 1. Non-limiting examples of influenza B HA amino acid sequences that comprise such mutations include the sequences of SEQ ID NOs: 13, 17, 21, 25, 29, 33 or 37.

Non-limiting examples of strains from which the influenza B HA might be derived are wt HA B/Singapore/INFKK-16-0569/2016 (EPI592707) (SEQ ID NO: 2), wt HA B/Washington/02/2019 (EPI1368874) (SEQ ID NO: 1), wt HA B/Rhode Island/01/2019 (EPI1383242) (SEQ ID NO: 3), wt HA B/Michigan/01/2021 (EPI1843974) (SEQ ID NO: 4), wt HA B/Henan-Xigong/1118/2021 (EPI1878454) (SEQ ID NO: 5), wt HA B/Austria/1359417/2021 (EPI1845793) (SEQ ID NO: 6), or wt HA B/Singapore/WUH4618/2021 (EPI1883660) (SEQ ID NO: 7) (see also Table 2).

In one aspect of the disclosure, the modified B HA may have at least the residue at position 402 modified, wherein the numbering is with respect to reference strain B/Washington/02/2019 (SEQ ID NO:1).

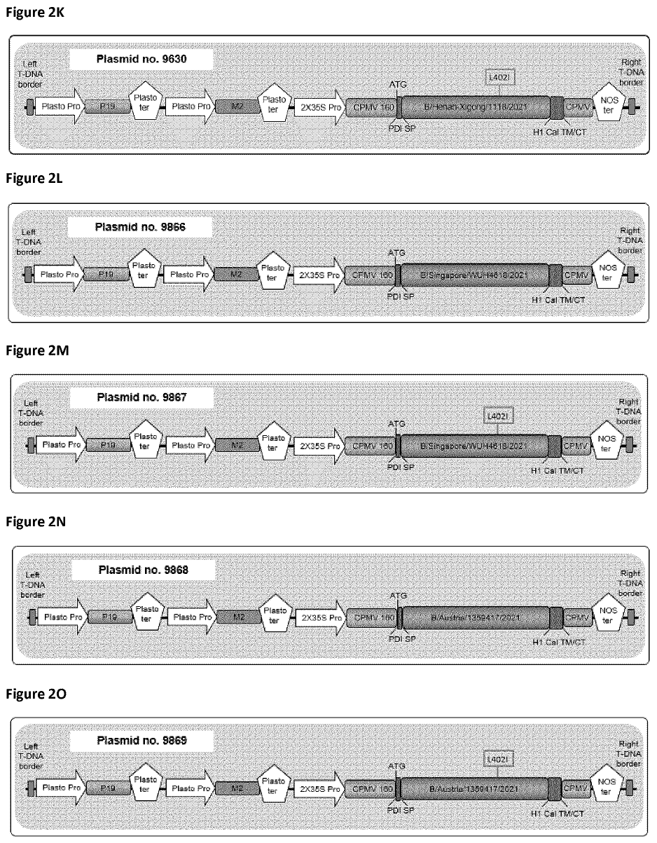

As shown in FIG. 3, modified B HA protein having the residue at position 402 changed from for example Leucine (L, Leu) to Isoleucine (I, Ile), hereinafter referred to as L402I, showed an increase of up to 2.3 fold in in planta yield as compared to a B HA that has Leucine (L, Leu) at this position (see also Example 3 and Table 4).

Modified HA from B/Singapore/0569/16 with the L404I substitution exhibited an approximate 2.3 fold increase in in planta yield when compared to parent B/Singapore/0569/16 HA (see FIG. 3).

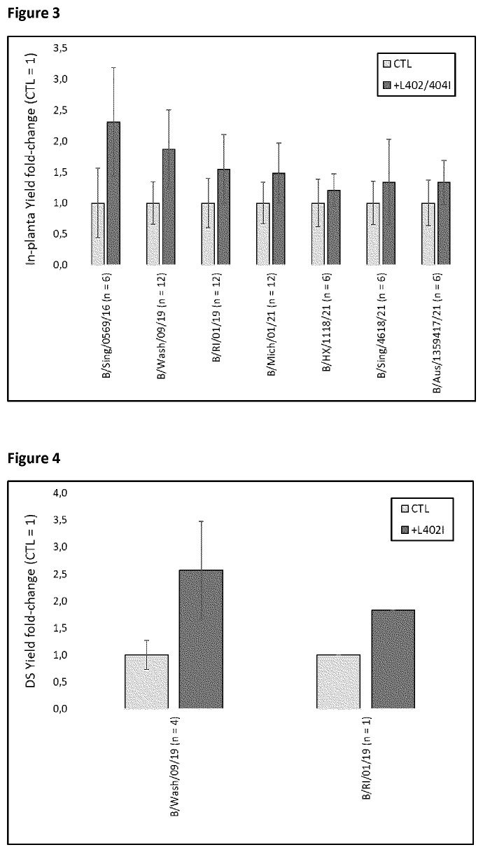

Modified HA from B/Washington/09/19 with the L402I substitution showed an approximate 1.9 fold increase in in planta yield when compared to parent B/Washington/09/19 HA (see FIG. 3). Furthermore, modified HA from B/Washington/09/19 with a L402I substitution exhibited an approximate 2.5 fold increase in drug substance (DS) yield fold change when compared to the unmodified B/Washington/09/19 HA protein (see FIG. 4).

Modified HA from B/Rhode Island/01/2019 with the L402I substitution exhibited an approximate 1.5 fold increase in in planta yield when compared to the unmodified B HA protein (see FIG. 3). The drug substance (DS) yield of modified B HA from B/Rhode Island/01/2019 was also increased by approximately 1.8 fold when compared to the unmodified B/Rhode Island/01/2019 HA protein (see FIG. 4).

Increased in planta yield were also observed for modified B HA from B/Michigan/01/2021 (1.5 fold change), B/Henan-Xigong/1118/2021 (approx. 1.5 fold change), B/Henan-Xigong/1118/2021 (approx. 1.2 fold change), B/Singapore/WUH4618/2021 (approx. 1.3 fold change) and B/Austria/1359417/2021 (approx. 1.3 fold change) (see FIG. 3, Example 3 and Table 4).

Without wishing to be bound by theory, it has been shown that an increase of HA yield in planta correlates to an increase of VLP production in planta.

In one aspect it is therefore provided that the residue at position 402 (numbering in accordance with B/Washington/09/19 numbering) of an influenza B HA may be modified to replace a Leucine (L, Leu) with an Isoleucine (I, Ile).

For example the modified B HA protein may have an amino acid sequence that has about 70, 75, 80, 85, 87, 90, 91, 92, 93 94, 95, 96, 97, 98, 99, 100% or any amount therebetween, sequence identity, or sequence similarity, with the amino acid sequence of SEQ ID NO: 13, SEQ ID NO: 17, SEQ ID NO: 21, SEQ ID NO: 25, SEQ ID NO: 29, SEQ ID NO: 33, or SEQ ID NO: 37, wherein the amino acid sequence has Isoleucine (I) or a conserved substitution of Isoleucine (I) that is not Leucine (L), for example Valine (V), Methionine (M) or Phenylalanine (F), at position 402 (numbering corresponding to reference strain B/Washington/09/19, SEQ ID NO: 1), wherein the modified B HA sequence does not occur naturally and wherein the HA proteins when expressed form VLP.

The present specification also provides a nucleic acid comprising a nucleotide sequence encoding a modified B HA with a substitution at position 402 as described above operatively linked to a regulatory region active in a plant.

For example the nucleotide sequences may have about 70, 75, 80, 85, 87, 90, 91, 92, 93 94, 95, 96, 97, 98, 99, 100% or any amount therebetween, sequence identity, or sequence similarity, with the nucleotide sequence encoding a B HA having amino acid sequence of SEQ ID NO: 13, SEQ ID NO: 17, SEQ ID NO: 21, SEQ ID NO: 25, SEQ ID NO: 29, SEQ ID NO: 33, or SEQ ID NO: 37, wherein the amino acid sequence has Isoleucine (I) or a conserved substitution of Isoleucine (I) that is not Leucine (L), for example Valine (V), Methionine (M) or Phenylalanine (F), at position 402 (numbering corresponding to reference strain B/Washington/09/19, SEQ ID NO: 1), wherein the modified B HA sequence does not occur naturally and wherein the HA proteins when expressed form VLP.

The nucleotide sequences may have about 70, 75, 80, 85, 87, 90, 91, 92, 93 94, 95, 96, 97, 98, 99, 100% or any amount therebetween, sequence identity, or sequence similarity, with the nucleotide sequence of SEQ ID NO: 12, 16, 20, 24, 28, 32 or 36, wherein the nucleotide codon that encode amino acid residue 402 of the modified B H, encodes Isoleucine (I) or a conserved substitution of Isoleucine (I) that is not Leucine (L), for example Valine (V), Methionine (M) or Phenylalanine (F) at position 402 (numbering corresponding to reference strain B/Washington/09/19, SEQ ID NO: 1) and wherein the modified B HA sequence does not occur naturally.

Influenza A viruses are divided into subtypes based on two proteins on the surface of the virus: hemagglutinin (H) and neuraminidase (N). Influenza A subtypes can be further broken down into different genetic “clades” and “sub-clades.”

Influenza B viruses are not divided into subtypes, but instead are classified into two co-circulating phylogenetically and antigenically distinct lineages, named after viruses B/Yamagata/16/88 (Yamagata-lineage) and B/Victoria/2/87 (Victoria-lineage). Similar to influenza A viruses, influenza B viruses can then be further classified into specific clades and sub-clades. Influenza B viruses generally change more slowly in terms of their genetic and antigenic properties than influenza A viruses.

Traditionally, different strains of influenza have been categorized based upon, e.g., the ability of influenza to agglutinate red blood cells (RBCs or erythrocytes). Antibodies specific for particular influenza strains may bind to the virus and, thus, prevent such agglutination. Assays determining strain types based on such inhibition are typically known as hemagglutinin inhibition assays (HI assays or HA1 assays) and are standard and well-known methods in the art to characterize influenza strains.

However, HA proteins from different virus strains also show significant sequence similarity at both the nucleic acid and amino acid levels. The level of similarity may vary between strains from different B lineages. This variation is sufficient to establish discrete lineages and the evolutionary lineage of the different strains, but the DNA and amino acid sequences of different strains are still readily aligned using conventional bioinformatics techniques (Langat, Pinky et al. PLoS pathogens 2017 December; vol. 13 (12)).

Multiple nucleotide sequences, or corresponding polypeptide sequences of hemagglutinin (HA), may be aligned to determine a “consensus” or “consensus sequence” of a subtype or lineage (see Gravel et al. iScience 24, November 2021). For example, the consensus sequence of the B HA2 domain is shown in the sequences of SEQ ID NO: 40 (the fusion peptide (FP) is in italics and the TMCT domain is underlined). By excluding the sequence of the FP and the sequence of the TMCT domain, the consensus sequence of the B HA2 ectodomain may be determined.

Therefore, it is also provided an influenza B HA protein with a modified HA2 ectodomain, wherein the HA2 ectodomain comprises one or more than one mutation, modification, or substitution in its amino acid sequence, wherein at least an amino acid that correspond with amino acids at positions 58 of SEQ ID NO:40 has been modified, when compared to the unmodified (parent) sequence. For example, Leucine (L) has been modified to a non-Leucine for example Isoleucine (I) at position 58 (L58I).

The modified B virus HA protein as described herewith, may therefore comprise a modified HA2 subunit (or HA2 domain) that comprises the following fusion peptide (in italics) and HA2 ectodomain sequence:

(SEQ ID NO: 41)
GFFGAIAGFLEGGWEGMIAGWHGYTSHGAHGVAVAADLKSTQEAINKITK
NLNSLSEX₁EVKNLQRLSGAMDELHNEIX₂ELDEKVDDLRADTISSQIEL
AVLLSNEGIINSEDEHLLALERKLKKMLGPX₃AVX₄IGNGCFETKHKCNQ
TCLDRIAAGTFX₅X₆GEFSLPTFDSLNITAASLNDDGLDNHTI

Wherein
X₁=I at position 58
X₂=L or I at position 78
X₃=S or absent at position 129
X₄=E or D at position 132
X₅=N or D at position 158
X₆=A or D at position 159.

In one aspect it is therefore provided a modified influenza B virus HA protein comprising a modified HA2 domain wherein the modified HA2 domain comprises at least one substitution when compared to the sequence of a parent (unmodified) or wildtype HA2 domain. The modified HA2 domain may have an amino acid sequences that has about 80, 82, 83, 85, 87, 90, 91, 92, 93 94, 95, 96, 97, 98, 99, 100% or any amount therebetween, sequence identity, or sequence similarity, with the amino acid sequence of SEQ ID NO: 41, wherein the sequence comprises a non-leucine, such for example an isoleucine at position 58. The modified influenza B virus HA protein may comprise an HA2 domain that comprises or consists of the sequence of SEQ ID NO: 41.

### Conservative Substitutions

As described herein, residues in B HA proteins may be identified and modified, substituted or mutated to produce modified B HA protein or B HA protein variants. The substitutions or mutations at specific positions are not limited to the amino acid substitutions described herewith or as given in the examples. For example, the B HA variants may contain conserved or conservative substitutions of described amino acid substitutions.

As used herein, the term “conserved substitution” or “conservative substitution” and grammatical variations thereof, refers to the presence of an amino acid residue in the sequence of the HA protein that is different from, but is in the same class of amino acid as the described substitution or described residue (i.e., a nonpolar residue replacing a nonpolar residue, an aromatic residue replacing an aromatic residue, a polar-uncharged residue replacing a polar-uncharged residue, a charged residue replacing a charged residue). In addition, conservative substitutions can encompass a residue having an interfacial hydropathy value of the same sign and generally of similar magnitude as the residue that is replacing the wildtype residue.

As used herein, the term “nonpolar residue” refers to glycine (G, Gly), alanine (A, Ala), valine (V, Val), leucine (L, Leu), isoleucine (I, Ile), and proline (P, Pro); the term “aromatic residue” refers to phenylalanine (F, Phe), tyrosine (Y, Tyr), and tryptophan (W, Trp); the term “polar uncharged residue” refers to serine (S, Ser), threonine (T, Thr), cysteine (C, Cys), methionine (M, Met), asparagine (N, Asn) and glutamine (Q, Gln); the term “charged residue” refers to the negatively charged amino acids aspartic acid (D, Asp) and glutamic acid (E, Glu), as well as the positively charged amino acids lysine (K, Lys), arginine (R, Arg), and histidine (H, His). Other classification of amino acids may be as follows:
amino acids with hydrophobic side chain (aliphatic): Alanine (A, Ala), Isoleucine (I, Ile), Leucine (L, Leu), Methionine (M, Met) and Valine (V, Val);
amino acids with hydrophobic side chain (aromatic): Phenylalanine (F, Phe), Tryptophan (W, Trp), Tyrosine (Y, Tyr);
amino acids with polar neutral side chain: Asparagine (N, Asn), Cysteine (C, Cys), Glutamine (Q, Gln), Serine (S, Ser) and Threonine (T, Thr);
amino acids with electrically charged side chains (acidic): Aspartic acid (D, Asp), Glutamic acid (E, Glu);
amino acids with electrically charged side chains (basic): Arginine (R, Arg); Histidine (H, His); Lysine (K, Lys), Glycine G, Gly) and Proline (P, Pro).

Conservative amino acid substitutions are likely to have a similar effect on the activity of the resultant HA protein variant or modified HA protein, as the original substitution or modification. Further information about conservative substitutions can be found, for instance, in Ben Bassat et al. (J. Bacteriol, 169:751-757, 1987), O'Regan et al. (Gene, 77:237-251, 1989), Sahin-Toth et al. (Protein ScL, 3:240-247, 1994), Hochuli et al (Bio/Technology, 6:1321-1325, 1988) and in widely used textbooks of genetics and molecular biology.

The Blosum matrices are commonly used for determining the relatedness of polypeptide sequences. The Blosum matrices were created using a large database of trusted alignments (the BLOCKS database), in which pairwise sequence alignments related by less than some threshold percentage identity were counted (Henikoff et al., Proc. Natl. Acad. Sci. USA, 89:10915-10919, 1992). A threshold of 90% identity was used for the highly conserved target frequencies of the BLOSUM90 matrix. A threshold of 65% identity was used for the BLOSUM65 matrix. Scores of zero and above in the Blosum matrices are considered “conservative substitutions” at the percentage identity selected. The following table shows exemplary conservative amino acid substitutions: Table 2.

TABLE 2
Exemplary conservative amino acid substitutions.

| | Very Highly - | | |
|---|---|---|---|
| Original | Conserved | Highly Conserved Substitutions | Conserved Substitutions (from the |
| Residue | Substitutions | (from the Blosum90 Matrix) | Blosum65 Matrix) |
| Ala | Ser | Gly, Ser, Thr | Cys, Gly, Ser, Thr, Val |
| Arg | Lys | Gln, His, Lys | Asn, Gln, Glu, His, Lys |
| Asn | Gln; His | Asp, Gln, His, Lys, Ser, Thr | Arg, Asp, Gln, Glu, His, Lys, Ser, Thr |
| Asp | Glu | Asn, Glu | Asn, Gln, Glu, Ser |
| Cys | Ser | None | Ala |
| Gln | Asn | Arg, Asn, Glu, His, Lys, Met | Arg, Asn, Asp, Glu, His, Lys, Met, Ser |
| Glu | Asp | Asp, Gln, Lys | Arg, Asn, Asp, Gln, His, Lys, Ser |
| Gly | Pro | Ala | Ala, Ser |
| His | Asn; Gln | Arg, Asn, Gln, Tyr | Arg, Asn, Gln, Glu, Tyr |
| Ile | Leu; Val | Leu, Met, Val | Leu, Met, Phe, Val |
| Leu | Ile; Val | Ile, Met, Phe, Val | Ile, Met, Phe, Val |
| Lys | Arg; Gln; Glu | Arg, Asn, Gln, Glu | Arg, Asn, Gln, Glu, Ser, |
| Met | Leu; Ile | Gln, Ile, Leu, Val | Gln, Ile, Leu, Phe, Val |
| Phe | Met; Leu; Tyr | Leu, Trp, Tyr | Ile, Leu, Met, Trp, Tyr |
| Ser | Thr | Ala, Asn, Thr | Ala, Asn, Asp, Gln, Glu, Gly, Lys, Thr |
| Thr | Ser | Ala, Asn, Ser | Ala, Asn, Ser, Val |
| Trp | Tyr | Phe, Tyr | Phe, Tyr |
| Tyr | Trp; Phe | His, Phe, Trp | His, Phe, Trp |
| Val | Ile; Leu | Ile, Leu, Met | Ala, Ile, Leu, Met, Thr |

The nucleotide sequence encoding the modified B HA protein may be optimized for human codon usage, for increased GC content, or a combination thereof. The modified HA protein may be expressed in a host or host cell, such for example in a plant, portion of a plant, or plant cell.

As described above, the parent sequence may be a wild-type sequence or the parent sequence may be a sequence that already comprise modifications (“parent modifications”) when compared to a wild-type sequence. The parent modification may be amino acid deletions or substitutions. For example, the parent modifications may comprise modifications such as deletions of the proteolytic cleavage site (also referred to as proteolytic loop). For example, the cleavage site and/or the fusion peptide or portions of the fusion peptide may be deleted to prevent cleavage of the HA protein. For example, the C-terminus of the H1 domain and the N-terminus of the HA2 domain, which comprise the cleavage side and fusion peptide may have been modified. For example the C-terminus of the H1 domain may comprise one or more than one deletion of an amino acid. Furthermore the N-terminal end of the Fusion Peptide domain of HA2 may comprise one or more than one deletion of amino acids 1 to 23 of SEQ ID NO: 41. For example, amino acids 1-11 of SEQ ID NO. 41 may have been deleted. Therefore, it is also provided a modified B virus HA protein that comprises a modified HA2 ectodomain, wherein the sequence of the modified HA2 ectodomain comprises amino acids 12 to 181 of SEQ ID NO: 41 or wherein the sequence of the modified HA2 ectodomain comprises the sequence of SEQ ID NO: 42. Accordingly, the modified HA2 ectodomain may have an amino acid sequences that has about 80, 82, 83, 85, 87, 90, 91, 92, 93 94, 95, 96, 97, 98, 99, 100% or any amount therebetween, sequence identity, or sequence similarity, with the amino acid sequence of SEQ ID NO: 42, wherein the sequence comprises a non-leucine, such for example an isoleucine at position 35. The modified influenza B virus HA protein may comprise an HA2 ectodomain that comprises or consists of the sequence of SEQ ID NO: 42.

The parent modifications may also comprise modifications of the transmembrane and cytoplasmic tail domain (TMCT). For example, the native TMCT in the parent sequence may be replaced with the TMCT from a different influenza HA than the parent HA.

Accordingly, the modified B HA protein, may comprise further modification, such as deletions or substitution compared to a wild type B HA. For example, the proteolytic cleavage site may be deleted or modified in the modified B HA protein, to prevent proteolytic cleavage of the HA0 precursor into the HA1 and HA2 subunits. The cleavage site is a prominent surface loop in the influenza HA protein and may for example be determined by sequence alignments or structural analysis of the HA protein (see for example Bertram et al. Reviews in Medical Virology, Volume 20, September 2010). Influenza HA proteins comprising a modified proteolytic cleavage site, as well as methods of producing influenza HA proteins comprising a modified proteolytic cleavage site are for example described in PCT application WO 2013/044390 and WO 2014/153674, which are hereby incorporated by reference.

Furthermore, the native transmembrane and cytoplasmic tail domain (TMCT) of the influenza B HA may be replaced with the TMCT of an influenza A HA. Therefore, the modified B HA may comprise a non-native TMCT. For example, the modified B HA may have the native TMCT replaced with the TMCT of influenza H1, H2, H3, H4, H5, H6, H7, H8, H9, H10, H11, H12, H13, H14, H15 or H16. In a preferred embodiment the TMCT in the modified B HA is a non-native TMCT from influenza HA H1 or H5. The replacement of the TMCT in influenza HA is for example described in PCT application WO 2010/148511, which is hereby incorporated by reference.

The modified influenza B HA may include a signal peptide to direct localization when expressed in the host or host cell. The signal peptide may be a native (with respect to the protein) signal or leader sequence, or a heterologous signal sequence. Therefore, as described herein, the modified influenza B protein may be produced as precursor protein comprising a modified influenza B protein and a heterologous amino acid signal peptide sequence. For example, the modified influenza B protein precursor may comprise the signal peptide from Protein disulphide isomerase (PDI SP; nucleotides 32-103 of Accession No. Z11499).

Modified influenza B HA protein as described herewith may further be incorporated into virus-like particles (VLPs). The term “virus-like particle” (VLP), or “virus-like particles” or “VLPs” refers to virus-like structures that are generally morphologically and antigenically similar to virions produced in an infection, but lack genetic information sufficient to replicate and thus are non-infectious. VLPs are structures that self-assemble and comprise one or more structural proteins such as for example modified influenza B HA protein. Therefore, the VLP may comprise modified influenza B HA protein. VLP may further comprise influenza virus protein, wherein the influenza virus protein consists of modified influenza B HA protein.

VLP may be produced in suitable host or host cells including plants and plant cells. Following extraction from the host or host cell and upon isolation and further purification under suitable conditions, VLP may be recovered as intact structures.

The VLP may be purified or extracted using any suitable method for example chemical or biochemical extraction. VLPs are relatively sensitive to desiccation, heat, pH, surfactants and detergents. Therefore, it may be useful to use methods that maximize yields, minimize contamination of the VLP fraction with cellular proteins, maintain the integrity of the proteins, or VLP, and, where required, the associated lipid envelope or membrane, methods of loosening the cell wall to release the proteins, or VLP. Minimizing or eliminating the use of detergence or surfactants such for example SDS or Triton™ X-100 may be beneficial for improving the yield of VLP extraction. VLP may be then assessed for structure and size by, for example, electron microscopy, or by size exclusion chromatography.

For enveloped viruses, such as influenza virus, it may be advantageous for a lipid layer or membrane to be retained by the virus. The composition, quality and quantity of the lipid may vary with the system (e.g. a plant-produced enveloped virus would include plant lipids or phytosterols in the envelope), and may contribute to an improved immune response.

Without wishing to be bound by theory, plant-made VLP comprising plant derived lipids, may induce a stronger immune reaction than VLP made in other manufacturing systems and the immune reaction induced by these plant-made VLP may be stronger when compared to the immune reaction induced by live or attenuated whole virus vaccines.

Furthermore, in addition to the potential adjuvant effect of the presence of plant lipids, the ability of plant N-glycans to facilitate the capture of glycoprotein antigens by antigen presenting cells, may be advantageous of the production of VLP in plants.

The VLP produced within a plant may comprise a modified influenza B HA protein comprising plant-specific N-glycans. Therefore, this disclosure also provides for a VLP comprising modified influenza B HA protein having plant specific N-glycans. Furthermore, it is provided VLP comprising plant lipids and modified influenza B HA protein having plant specific N-glycans.

Furthermore, it is provided a method of producing VLPs that comprise a modified B HA as described above in a host or host cell such for example a plant.

The method may involve introducing a nucleic acid encoding a modified B HA with a substitution at position 402 (numbering corresponding to reference strain B/Washington/09/19, SEQ ID NO: 1) operatively linked to a regulatory region active in the host or host cell, and incubating the host or host cell under conditions that permit the expression of the nucleic acid, thereby producing the VLPs. The method may also involve introducing a nucleic acid encoding an influenza B virus HA protein comprising a modified HA2 ectodomain as described herewith operatively linked to a regulatory region active in the host or host cell, and incubating the host or host cell under conditions that permit the expression of the nucleic acid, thereby producing the VLPs.

In addition, it is provided a method of increasing yield of VLPs that comprise a modified B HA with a substitution at position 402 (numbering corresponding to reference strain B/Washington/09/19, SEQ ID NO: 1) as described above in a host or host cell. The method involves introducing a nucleic acid encoding a modified B HA with a substitution at position 402 (numbering corresponding to reference strain B/Washington/09/19, SEQ ID NO: 1) operatively linked to a regulatory region active in the host or host cell and incubating the host or host cell under conditions that permit the expression of the nucleic acid, thereby producing the VLPs.

Furthermore, it is provided a method of increasing yield of VLPs that comprise influenza B virus HA protein comprising a modified HA2 ectodomain as described above in a host or host cell. The method involves introducing a nucleic acid encoding influenza B virus HA protein comprising a modified HA2 ectodomain operatively linked to a regulatory region active in the host or host cell and incubating the host or host cell under conditions that permit the expression of the nucleic acid, thereby producing the VLPs.

The present specification further provides for a VLP comprising a B HA with a substitution at position 402 and/or VLP comprising influenza B virus HA protein comprising a modified HA2 ectodomain as described herewith. The VLP may be produced by the method as provided by the present disclosure. The VLP comprising the modified B HA show improved characteristics when compared to VLPs that comprise the unmodified B HA protein.

Also provided herein are methods of increasing production or yield of VLPs comprising modified influenza B HA in plants. For example, a method may involve introducing a nucleic acid encoding a modified influenza B HA, as described herein, into the plant, portion of the plant, or plant cell. The nucleic acid encoding the modified influenza B HA may be optimized for human codon usage, increased GC content, or a combination thereof. One or more than one modified influenza B HA protein may be expressed in a plant, portion of the plant, or plant cell, in order to produce a VLP comprising one or more than one modified influenza B HA protein. Alternatively, the method may comprise providing a plant, portion of the plant, or plant cell that comprises the nucleic acid encoding the modified influenza B HA protein in order to produce a VLP comprising the one or more than one modified influenza B HA protein.

The methods of producing a VLP comprising a modified influenza B HA may further comprise a step of introducing a second nucleic acid sequence into the plant, portion of the plant, or plant cell, wherein the second nucleic acid encodes a proton channel protein that is co-expressed with the modified influenza B HA. For example, the proton channel protein may be an influenza A subtype M2 protein, such as A/New Caledonia/20/99 M2. The co-expression of the proton channel protein may lead to an increased accumulation of modified influenza B HA protein and/or VLP comprising the modified influenza HA protein as for example described in WO 2013/044390 which is incorporated herein by reference.

By “co-expression”, it is meant the introduction and expression of two or more nucleotide sequences, each of the two or more nucleotide sequences encoding a protein of interest, or a fragment of a protein of interest within a plant, portion of a plant or a plant cell. The two or more nucleotide sequences may be introduced into the plant, portion of the plant or the plant cell within one vector, so that each of the two or more nucleotide sequences is under the control of a separate regulatory region (e.g. comprising a dual construct). Alternatively, the two or more nucleotide sequences may be introduced into the plant, portion of the plant or the plant cell within separate vectors (e.g. comprising single constructs), and each vector comprising appropriate regulatory regions for the expression of the corresponding nucleic acid. For example, two nucleotide sequences, each on a separate vector and introduced into separate Agrobacterium tumefaciens hosts, may be co-expressed by mixing suspensions of each A. tumefaciens host in a desired volume (for example, an equal volume, or the ratios of each A. tumefaciens host may be altered) before vacuum infiltration. In this manner, co-infiltration of multiple A. tumefaciens suspensions permits co-expression of multiple transgenes.

The current disclosure further provides a drug substance (DS) comprising, as the desired product, modified influenza B HA protein as described above, said drug substance being substantially free of product related impurities, wherein the impurities are not immune-active. A preferred drug substance is further substantially free of process related impurities.

Within the context of the present application, the term “drug substance” refers to a product or active ingredients suitable for use as i) the active principle of a medicament or drug product, ii) an active pharmaceutical ingredient of a medicament or drug product iii) a bulk purified active principle of a medicament or drug product or iv) a bulk purified active ingredient of a medicament or drug product. The medicament or drug product may be a vaccine.

It is therefore further provided a drug substance (DS) comprising immune-active modified influenza B HA protein. Higher yield of DS is obtained from host cells expressing the modified influenza B HA compared to a DS that has been obtained from a host expressing an unmodified influenza B HA protein (see FIG. 4). Therefore, it is also provided a method of increasing the yield of a DS obtained from a host or host cell that expresses the modified influenza B HA protein compared to the yield of a DS that has been obtained from a host that expresses unmodified (parent) S protein.

It is therefore further provided a drug substance (DS) comprising immune-active modified influenza B HA protein.

The modified influenza B HA protein may self-assemble into virus-like particle (VLP). Accordingly, it is also provided a DS comprising VLP comprising modified influenza B HA protein.

In a further aspect, it is also provided a Drug Product (also referred to as pharmaceutical formulation or pharmaceutical composition). The Drug Product may be formulated as a finished dosage form, for example as a solution, capsule or tablet. The Drug Product comprises the Drug Substance. The Drug Product may further comprise other ingredients such for example pharmaceutically acceptable carriers and/or excipient, such as buffer system, adjuvants, preservatives, tonicity agent(s), chelating agent(s), antiadherents, vehicles etc. Pharmaceutical acceptable carrier and excipient are well known within the art. Therefore, it is also provided a drug product, pharmaceutical formulation or pharmaceutical composition comprising pharmaceutically acceptable carriers and/or excipient, and VLP, the VLP comprising modified influenza B HA protein or the VLP comprising viral protein, wherein the viral protein consists of modified influenza B HA protein.

When one or more than one modified influenza B HA protein is expressed in a host or host cell, the one or more than one modified influenza B HA proteins self-assemble into VLPs. The host or host cell may be harvested under suitable extraction and purification conditions to maintain the integrity of the VLP, and the VLP comprising the one or more than one mutant influenza HA may be purified.

The present disclosure also provides the use of a modified influenza B HA, VLP or DS comprising the modified influenza B HA, as described herein, for inducing an immune response or for inducing immunity to an influenza infection in a subject. Also disclosed herein is an antibody or antibody fragment, prepared by administering the modified influenza B HA, VLP or DS comprising the modified influenza B HA, to a subject or a host animal. Further provided is a composition comprising an effective dose of a modified influenza B HA, VLP or DS comprising the modified influenza B HA, as described herein, and a pharmaceutically acceptable carrier, adjuvant, vehicle, or excipient, for inducing an immune response in a subject. Also provided is a vaccine for inducing an immune response in a subject, wherein the vaccine comprises an effective dose of the modified influenza B HA.

Further provided is a composition comprising an effective dose of modified influenza B HA protein, VLP or DS comprising the modified influenza B HA protein, as described herein, and a pharmaceutically acceptable carrier, adjuvant, vehicle, or excipient, for inducing an immune response in a subject. Also provided is a vaccine for inducing an immune response again influenza in a subject, wherein the vaccine comprises an effective dose of the modified influenza B HA protein, VLP or DS comprising the modified influenza B HA protein.

The composition or vaccine may comprise VLP comprising influenza HA protein, wherein the HA protein is derived from the same influenza type, subtype, lineage, subgenera or strain, or the composition or vaccine may comprise multiple VLP types, wherein each VLP type comprises HA protein, wherein the HA protein may be derived from different influenza type, subtype, lineage, subgenera or strain i.e. the composition or vaccine may comprise a mixture of different influenza VLP. For example, the composition or vaccine may comprise a first VLP comprising a first influenza HA protein from a first influenza subtype, lineage or strain and a second VLP comprising a second influenza HA protein from a second influenza subtype, lineage or strain. Furthermore, the composition may also comprise a third VLP comprising a third influenza HA protein from a third influenza subtype, lineage or strain and/or the composition or vaccine may comprise a fourth VLP comprising a fourth influenza HA protein from a fourth influenza subtype, lineage, subgenera or strain.

The composition or vaccine may further comprise VLP comprising HA protein from more than one type of HA subtype, lineage or strain. For example, the VLP may comprise a first modified B HA protein from a first B HA lineage or strain and a second HA protein, wherein the second HA protein is derived from a HA from a second B lineage or strain or the second HA is derived from a HA from an influenza A subtype or strain. Furthermore, the VLP may comprise a third HA protein, wherein the third HA is derived from a third B lineage or strain or the third HA is derived from a HA from an influenza A subtype or strain and/or the VLP may comprise a fourth HA, wherein the fourth HA is derived from a fourth B lineage or strain or the fourth HA is derived from a HA from an influenza A subtype or strain.

Accordingly, the description also provides compositions or vaccines that are monovalent (univalent), or multivalent (polyvalent). The monovalent composition or vaccine may immunize a subject against a single type of influenza strain, whereas the multivalent composition or vaccine may immunize a subject against more than one influenza strain. For example, the composition or vaccine may be a bivalent composition or vaccine, which upon administration, may immunize a subject against two different types of influenza families, sub-groups, types, subtypes, lineages or strains. Furthermore, the composition or vaccine may be a trivalent composition, or the vaccine or composition may be a tetravalent or quadrivalent composition or vaccine. Furthermore, the vaccine may also be multivalent with respect to different types of viruses. For example, the vaccine may immunize a subject against one or more than one influenza strain (first type of virus) and against a second type of virus for example coronavirus.

Accordingly, it is also provided a multivalent immunogenic composition comprising two or more than two types of VLP, wherein at least one type of VLP (first type of VLP) comprises modified B HA protein as described herewith (first modified B HA). The multivalent immunogenic composition may further comprise a second type of VLP that also comprises modified B HA protein (second modified B HA) as described herewith, wherein the first and second type VLP comprise modified B HA proteins that are derived from different influenza B viruses. For example, the first and second VLP may comprise modified B HA protein that belong to different influenza B lineages, respectively. The multivalent immunogenic composition may further comprise one or more than one type of VLP that comprise influenza A HA protein. For example, the influenza A HA may be derived from influenza subtype H1 and/or from influenza subtype H3.

It is also provided a quadrivalent immunogenic composition comprising a first type VLP comprising the modified influenza B HA as described herewith, a second type VLP comprising the modified influenza B HA as described herewith, a third type VLP comprising influenza A HA and a fourth type VLP comprising influenza A HA, wherein the first type VLP comprises modified B HA that are derived from a different influenza B lineage than the modified B HA of the second type of VLP. For example the modified B HA in the first type VLP may be derived from the B/Victoria lineage and the modified B HA in second type VLP may be derived from the B/Yamagata lineage. Furthermore, the influenza A HA of the third type VLP may be derived from a different influenza A subtype than the influenza A HA of the fourth type of VLP. For example, the influenza A HA in the third type VLP may be derived from influenza H3 and the influenza A HA in the fourth type VLP may be derived from influenza H1.

The monovalent or multivalent composition or vaccine may further comprise a pharmaceutically acceptable carrier, adjuvant, vehicle, or excipient, for inducing an immune response in a subject.

Adjuvant systems to enhance a subject's immune response to a vaccine antigen are well known and may be used in conjunction with the vaccine or pharmaceutical composition as described herewith. There are many types of adjuvants that may be used. Common adjuvants for human use are aluminum hydroxide, aluminum phosphate and calcium phosphate. There are also a number of adjuvants based on oil emulsions (oil in water or water in oil emulsions such as Freund's incomplete adjuvant (FIA), Montanide™, Adjuvant 65, and Lipovant™), products from bacterial (or their synthetic derivatives), endotoxins, fatty acids, paraffinic, or vegetable oils, cholesterols, and aliphatic amines or natural organic compounds such for example squalene. Non-limiting adjuvants that might be used include for example oil-in water emulsions of squalene oil (for example MF-59 or AS03), adjuvant composed of the synthetic TLR4 agonist glucopyranosyl lipid A (GLA) integrated into stable emulsion (SE) (GLA-SE) or CpG 1018 a toll-like receptor (TLR9) agonist adjuvant.

Therefore, the vaccine or pharmaceutical composition may comprise one or more than one adjuvant. For example, the vaccine or pharmaceutical composition may comprise aluminum hydroxide, aluminum phosphate, calcium phosphate, an oil in water or water in oil emulsions, an emulsion comprising squalene (for example MF-59 or AS03), an emulsion comprising GLA-SE, or CpG 1018.

Also provided herein are methods for inducing an immune response or inducing immunity to an influenza infection in a subject comprising of administering the modified influenza B HA or VLP comprising the modified influenza B HA, to a subject orally, intranasally, intramuscularly, intraperitoneally, intravenously, or subcutaneously.

Influenza B HA proteins or modified influenza B HA proteins as disclosed herein, include any known HA proteins derived from any known influenza B strain, but also modifications to known influenza B strains that develop over time. For example, influenza HA may be derived from B/Washington/02/2019 (EPI1368874), B/Singapore/INFKK-16-0569/2016 (EPI592707), B/Rhode Island/01/2019 (EPI1383242), B/Michigan/01/2021 (EPI1843974), B/Henan-Xigong/1118/2021 (EPI1878454), B/Austria/1359417/2021 (EPI1845793), or B/Singapore/WUH4618/2021 (EPI1883660). Influenza B HA may include HA derived from strains, wherein the HA has about 30-100%, or any amount therebetween, amino acid sequence identity to any HA derived from the influenza B strains listed above, provided that the influenza HA protein comprises at least one substitution as described herewith and is able to form VLPs, induces an immune response when administered to a subject, induces hemagglutination or a combination thereof.

For example, influenza HA proteins may have 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100%, or any amount therebetween, amino acid sequence identity (sequence similarity, percent identity, percent similarity) to any HA derived from the influenza B strains listed above and comprises at least one substitution as described herewith and is able to form VLPs, induces an immune response when administered to a subject, induces hemagglutination or a combination thereof.

The terms “percent similarity”, “sequence similarity”, “percent identity”, or “sequence identity”, when referring to a particular sequence, are used for example as set forth in the University of Wisconsin GCG software program, or by manual alignment and visual inspection (see, e.g., Current Protocols in Molecular Biology, Ausubel et al., eds. 1995 supplement). Methods of alignment of sequences for comparison are well-known in the art. Optimal alignment of sequences for comparison can be conducted, using for example the algorithm of Smith & Waterman, (1981, Adv. Appl. Math. 2:482), by the alignment algorithm of Needleman & Wunsch, (1970, J. Mol. Biol. 48:443), by the search for similarity method of Pearson & Lipman, (1988, Proc. Natl. Acad. Sci. USA 85:2444), by computerized implementations of these algorithms (for example: GAP, BESTFIT, FASTA, and TFASTA in the Wisconsin Genetics Software Package, Genetics Computer Group (GCG), 575 Science Dr., Madison, Wis.).

An example of an algorithm suitable for determining percent sequence identity and sequence similarity are the BLAST and BLAST 2.0 algorithms, which are described in Altschul et al., (1977, Nuc. Acids Res. 25:3389-3402) and Altschul et al., (1990, J. Mol. Biol. 215:403-410), respectively. BLAST and BLAST 2.0 are used, with the parameters described herein, to determine percent sequence identity for the nucleic acids and proteins of the invention. For example, the BLASTN program (for nucleotide sequences) may use as defaults a wordlength (W) of 11, an expectation (E) of 10, M=5, N=−4 and a comparison of both strands. For amino acid sequences, the BLASTP program may use as defaults a word length of 3, and expectation (E) of 10, and the BLOSUM62 scoring matrix (see Henikoff & Henikoff, 1989, Proc. Natl. Acad. Sci. USA 89:10915) alignments (B) of 50, expectation (E) of 10, M=5, N=−4, and a comparison of both strands. Software for performing BLAST analyses is publicly available through the National Center for Biotechnology Information (see URL: ncbi.nlm.nih.gov/).

Influenza B HA protein includes any HA protein comprising an amino acid sequence having from about 30 to about 100%, from about 40 to about 100%, from about 50 to about 100%, from about 60 to about 100%, from about 70 to about 100%, from about 80 to about 100%, from about 85 to about 100%, from about 90 to about 100%, from 95 to about 100%, or from about 97 to about 100% from about 98 to about 100%, or any amount therebetween, sequence identity or sequence similarity with influenza B HA sequence from a wt HA B/Singapore/INFKK-16-0569/2016 (EPI592707) (SEQ ID NO: 2), wt HA B/Washington/02/2019 (EPI1368874) (SEQ ID NO: 1), wt HA B/Rhode Island/01/2019 (EPI1383242) (SEQ ID NO: 3), wt HA B/Michigan/01/2021 (EPI1843974) (SEQ ID NO: 4), wt HA B/Henan-Xigong/1118/2021 (EPI1878454) (SEQ ID NO: 5), wt HA B/Austria/1359417/2021 (EPI1845793) (SEQ ID NO: 6), and wt HA B/Singapore/WUH4618/2021 (EPI1883660) (SEQ ID NO: 7), provided that the influenza HA protein comprises at least one substitution as described herewith and is able to form VLPs, induces an immune response when administered to a subject, induces hemagglutination or a combination thereof.

Furthermore the modified influenza HA protein includes any HA protein comprising an amino acid sequence having from about 30% to about 100%, from about 40% to about 100%, from about 50% to about 100%, from about 60% to about 100%, from about 70% to about 100%, from about 80% to about 100%, from about 85% to about 100%, from about 90% to about 100%, from 95% to about 100%, or from about 97% to about 100% from about 98% to about 100%, or any amount therebetween, sequence identity or sequence similarity with a sequence of the sequences of SEQ ID NO: 13, SEQ ID NO: 17, SEQ ID NO: 21, SEQ ID NO: 25, SEQ ID NO: 29, SEQ ID NO: 33, or SEQ ID NO: 37, provided that the influenza HA protein comprises at least one substitution as described herewith and is able to form VLPs, induces an immune response when administered to a subject, induces hemagglutination or a combination thereof.

As described herein, one or more than one specific mutation or modification in influenza B HA results in increased accumulation of HA protein and increased VLP production in plants, as compared to unmodified influenza HA.

Examples of modified influenza B HA proteins having enhanced influenza HA and/or VLP production in plants include, but are not limited to the following:
L404I B/Singapore/INFKK-16-0569/2016 Mutant HA (Construct #8894, SEQ ID NO:13),
L402I B/Washington/02/2019 Mutant HA (Construct #8881, SEQ ID NO: 17),
L402I B/Rhode Island/01/2019 Mutant HA (Construct #7787, SEQ ID NO: 21),
L402I B/Michigan/01/2021 Mutant HA (Construct #9628, SEQ ID NO: 25),
L402I B/Henan-Xigong/1118/2021 Mutant HA (Construct #9630, SEQ ID NO: 29),
L402I B/Singapore/WUH4618/2021 Mutant HA (Construct #9867, SEQ ID NO: 33) and
L402I B/Austria/1359417/2021 Mutant HA (Construct #9869, SEQ ID NO: 37).

One or more than one modified genetic construct comprising the modified B HA protein of the present description may be expressed in any suitable host or host cell that is transformed by the nucleic acids, or nucleotide sequence, or constructs, or vectors of the present disclosure. The host or host cell may be from any source including plants, fungi, bacteria, insect and animals for example mammals. Therefore, the host or host cell may be selected from a plant or plant cell, a fungi or a fungi cell, a bacteria or bacteria cell, an insect or an insect cell, and animal or an animal cell. The mammal or animal may not be a human. Accordingly, the host or host cell, may be a non-human host or host cell. In a preferred embodiment the host or host cell is a plant, portion of a plant or plant cell.

The term “plant”, “portion of a plant”, “plant portion’, “plant matter”, “plant biomass”, “plant material”, “plant extract”, or “plant leaves”, as used herein, may comprise an entire plant, tissue, cells, or any fraction thereof, intracellular plant components, extracellular plant components, liquid or solid extracts of plants, or a combination thereof, that are capable of providing the transcriptional, translational, and post-translational machinery for expression of one or more than one nucleic acids described herein, and/or from which an expressed protein or VLP may be extracted and purified. Plants may include, but are not limited to, herbaceous plants. Furthermore plants may include, but are not limited to, agricultural crops including for example canola, Brassica spp., maize, Nicotiana spp., (tobacco) for example, Nicotiana benthamiana, Nicotiana rustica, Nicotiana, tabacum, Nicotiana alata, Arabidopsis thaliana, alfalfa, potato, sweet potato (Ipomoea batatus), ginseng, pea, oat, rice, soybean, wheat, barley, sunflower, cotton, corn, rye (Secale cereale), sorghum (Sorghum bicolor, Sorghum vulgare), safflower (Carthamus tinctorius).

The term “plant portion”, as used herein, refers to any part of the plant including but not limited to leaves, stem, root, flowers, fruits, a plant cell obtained from leaves, stem, root, flowers, fruits, a plant extract obtained from leaves, stem, root, flowers, fruits, or a combination thereof. The term “plant extract”, as used herein, refers to a plant-derived product that is obtained following treating a plant, a portion of a plant, a plant cell, or a combination thereof, physically (for example by freezing followed by extraction in a suitable buffer), mechanically (for example by grinding or homogenizing the plant or portion of the plant followed by extraction in a suitable buffer), enzymatically (for example using cell wall degrading enzymes), chemically (for example using one or more chelators or buffers), or a combination thereof. A plant extract may be further processed to remove undesired plant components for example cell wall debris. A plant extract may be obtained to assist in the recovery of one or more components from the plant, portion of the plant or plant cell, for example a protein (including protein complexes, protein surprastructures and/or VLPs), a nucleic acid, a lipid, a carbohydrate, or a combination thereof from the plant, portion of the plant, or plant cell. If the plant extract comprises proteins, then it may be referred to as a protein extract. A protein extract may be a crude plant extract, a partially purified plant or protein extract, or a purified product, that comprises one or more proteins, protein complexes, protein suprastructures, and/or VLPs, from the plant tissue. If desired, a protein extract or a plant extract may be partially purified using techniques known to one of skill in the art. For example, the extract may be subjected to salt or pH precipitation, centrifugation, gradient density centrifugation, filtration, chromatography, for example, size exclusion chromatography, ion exchange chromatography, affinity chromatography, or a combination thereof. A protein extract may also be purified, using techniques that are known to one of skill in the art.

The nucleic acid encoding a modified influenza B HA as described herein may further comprise sequences that enhance expression of the modified influenza B HA in a plant, portion of the plant, or plant cell. Sequences that enhance expression may include, for example, plant-derived expression enhancer or plant-virus derived expression enhancer. The expression enhancer may be in operative association with the nucleic acid encoding the modified influenza hemagglutinin (HA) protein. The sequence encoding the modified influenza hemagglutinin (HA) may also be optimized for human codon usage, increased GC content, or a combination thereof.

The term “plant-derived expression enhancer”, as used herein, refers to a nucleotide sequence obtained from a plant, the nucleotide sequence encoding a 5′UTR. Examples of a plant derived expression enhancer are described in WO 2019/173924 or WO 2020/181354. The plant derived expression enhancer may be used within a plant expression system comprising a regulatory region that is operatively linked with the plant-derived expression enhancer sequence and a nucleotide sequence of interest.

Furthermore, sequences that enhance expression may include plant-virus derived expression enhancer, for example cowpea mosaic virus (CPMV) enhancer element.

The term “CPMV enhancer element”, as used herein, refers to a nucleotide sequence encoding the 5′UTR regulating the Cowpea Mosaic Virus (CPMV) RNA2 polypeptide or a modified CPMV sequence as is known in the art. For example, a CPMV enhancer element or a CPMV expression enhancer, includes a nucleotide sequence as described in WO2015/14367; WO2015/103704; WO2007/135480; WO2009/087391; Sainsbury F., and Lomonossoff G. P., (2008, Plant Physiol. 148: pp. 1212-1218), each of which is incorporated herein by reference. A CPMV enhancer sequence can enhance expression of a downstream heterologous open reading frame (ORF) to which they are attached. The CPMV expression enhancer may include CPMV HT, CPMVX (where X=160, 155, 150, 114), for example CPMV 160, CPMVX+ (where X=160, 155, 150, 114), for example CPMV 160+, CPMV-HT+, CPMV HT+[WT115], or CPMV HT+ (WO2015/143567; WO2015/103704 which are incorporated herein by reference). In a preferred embodiment the CPMV expression enhance is CPMV 160. The CPMV expression enhancer may be used within a plant expression system comprising a regulatory region that is operatively linked with the CPMV expression enhancer sequence and a nucleotide sequence of interest for example the nucleotide sequence encoding the modified B HA of the present disclosure.

By “operatively linked” it is meant that the particular sequences interact either directly or indirectly to carry out an intended function, such as mediation or modulation of expression of a nucleic acid sequence. The interaction of operatively linked sequences may, for example, be mediated by proteins that interact with the operatively linked sequences.

The term “construct”, “vector” or “expression vector”, as used herein, refers to a recombinant nucleic acid for transferring exogenous nucleic acid sequences into host cells (e.g. plant cells) and directing expression of the exogenous nucleic acid sequences in the host cells. “Expression cassette” refers to a nucleotide sequence comprising a nucleic acid of interest under the control of, and operably (or operatively) linked to, an appropriate promoter or other regulatory elements for transcription of the nucleic acid of interest in a host cell. As one of skill in the art would appreciate, the expression cassette may comprise a termination (terminator) sequence that is any sequence that is active the plant host. For example, the termination sequence may be derived from the RNA-2 genome segment of a bipartite RNA virus, e.g. a comovirus, the termination sequence may be a NOS terminator, or terminator sequence may be obtained from the 3′UTR of the alfalfa plastocyanin gene.

The constructs of the present disclosure may further comprise a 3′ untranslated region (UTR). A 3′ untranslated region contains a polyadenylation signal and any other regulatory signals capable of effecting mRNA processing or gene expression. The polyadenylation signal is usually characterized by effecting the addition of polyadenylic acid tracks to the 3′ end of the mRNA precursor. Polyadenylation signals are commonly recognized by the presence of homology to the canonical form 5′ AATAAA-3′ although variations are not uncommon. Non-limiting examples of suitable 3′ regions are the 3′ transcribed non-translated regions containing a polyadenylation signal of Agrobacterium tumor inducing (Ti) plasmid genes, such as the nopaline synthase (Nos gene) and plant genes such as the soybean storage protein genes, the small subunit of the ribulose-1, 5-bisphosphate carboxylase gene (ssRUBISCO; U.S. Pat. No. 4,962,028; which is incorporated herein by reference), the promoter used in regulating plastocyanin expression.

By “regulatory region” “regulatory element” or “promoter” it is meant a portion of nucleic acid typically, but not always, upstream of the protein coding region of a gene, which may be comprised of either DNA or RNA, or both DNA and RNA. When a regulatory region is active, and in operative association, or operatively linked, with a nucleotide sequence of interest, this may result in expression of the nucleotide sequence of interest. A regulatory element may be capable of mediating organ specificity, or controlling developmental or temporal gene activation. A “regulatory region” includes promoter elements, core promoter elements exhibiting a basal promoter activity, elements that are inducible in response to an external stimulus, elements that mediate promoter activity such as negative regulatory elements or transcriptional enhancers. “Regulatory region”, as used herein, also includes elements that are active following transcription, for example, regulatory elements that modulate gene expression such as translational and transcriptional enhancers, translational and transcriptional repressors, upstream activating sequences, and mRNA instability determinants. Several of these latter elements may be located proximal to the coding region.

In the context of this disclosure, the term “regulatory element” or “regulatory region” typically refers to a sequence of DNA, usually, but not always, upstream (5′) to the coding sequence of a structural gene, which controls the expression of the coding region by providing the recognition for RNA polymerase and/or other factors required for transcription to start at a particular site. However, it is to be understood that other nucleotide sequences, located within introns, or 3′ of the sequence may also contribute to the regulation of expression of a coding region of interest. An example of a regulatory element that provides for the recognition for RNA polymerase or other transcriptional factors to ensure initiation at a particular site is a promoter element. Most, but not all, eukaryotic promoter elements contain a TATA box, a conserved nucleic acid sequence comprised of adenosine and thymidine nucleotide base pairs usually situated approximately 25 base pairs upstream of a transcriptional start site. A promoter element may comprise a basal promoter element, responsible for the initiation of transcription, as well as other regulatory elements that modify gene expression.

There are several types of regulatory regions, including those that are developmentally regulated, inducible or constitutive. A regulatory region that is developmentally regulated, or controls the differential expression of a gene under its control, is activated within certain organs or tissues of an organ at specific times during the development of that organ or tissue. However, some regulatory regions that are developmentally regulated may preferentially be active within certain organs or tissues at specific developmental stages, they may also be active in a developmentally regulated manner, or at a basal level in other organs or tissues within the plant as well. Examples of tissue-specific regulatory regions, for example see-specific a regulatory region, include the napin promoter, and the cruciferin promoter (Rask et al., 1998, J. Plant Physiol. 152:595-599; Bilodeau et al., 1994, Plant Cell 14:125-130). An example of a leaf-specific promoter includes the plastocyanin promoter (see U.S. Pat. No. 7,125,978, which is incorporated herein by reference).

An inducible regulatory region is one that is capable of directly or indirectly activating transcription of one or more DNA sequences or genes in response to an inducer. In the absence of an inducer the DNA sequences or genes will not be transcribed. Typically, the protein factor that binds specifically to an inducible regulatory region to activate transcription may be present in an inactive form, which is then directly or indirectly converted to the active form by the inducer. However, the protein factor may also be absent. The inducer can be a chemical agent such as a protein, metabolite, growth regulator, herbicide or phenolic compound or a physiological stress imposed directly by heat, cold, salt, or toxic elements or indirectly through the action of a pathogen or disease agent such as a virus. A plant cell containing an inducible regulatory region may be exposed to an inducer by externally applying the inducer to the cell or plant such as by spraying, watering, heating or similar methods. Inducible regulatory elements may be derived from either plant or non-plant genes (e.g. Gatz, C. and Lenk, I. R. P., 1998, Trends Plant Sci. 3, 352-358). Examples, of potential inducible promoters include, but not limited to, tetracycline-inducible promoter (Gatz, C., 1997, Ann. Rev. Plant Physiol. Plant Mol. Biol. 48, 89-108), steroid inducible promoter (Aoyama, T. and Chua, N. H., 1997, Plant J. 2, 397-404) and ethanol-inducible promoter (Salter, M. G., et al, 1998, Plant Journal 16, 127-132; Caddick, M. X., et al, 1998, Nature Biotech. 16, 177-180) cytokinin inducible IB6 and CKI1 genes (Brandstatter, I. and Kieber, J. J., 1998, Plant Cell 10, 1009-1019; Kakimoto, T., 1996, Science 274, 982-985) and the auxin inducible element, DR5 (Ulmasov, T., et al., 1997, Plant Cell 9, 1963-1971).

A constitutive regulatory region directs the expression of a gene throughout the various parts of a plant and continuously throughout plant development. Examples of known constitutive regulatory elements include promoters associated with the CaMV 35S transcript. (p35S; Odell et al., 1985, Nature, 313:810-812; which is incorporated herein by reference), the rice actin 1 (Zhang et al, 1991, Plant Cell, 3:1155-1165), actin 2 (An et al., 1996, Plant J., 10:107-121), or tms 2 (U.S. Pat. No. 5,428,147), and triosephosphate isomerase 1 (Xu et. al., 1994, Plant Physiol. 106:459-467) genes, the maize ubiquitin 1 gene (Cornejo et al, 1993, Plant Mol. Biol. 29:637-646), the Arabidopsis ubiquitin 1 and 6 genes (Holtorf et al, 1995, Plant Mol. Biol. 29:637-646), the tobacco translational initiation factor 4A gene (Mandel et al, 1995 Plant Mol. Biol. 29:995-1004), the Cassava Vein Mosaic Virus promoter, pCAS, (Verdaguer et al., 1996); the promoter of the small subunit of ribulose biphosphate carboxylase, pRbcS: (Outchkourov et al., 2003), the pUbi (for monocots and dicots).

The term “constitutive” as used herein does not necessarily indicate that a nucleotide sequence under control of the constitutive regulatory region is expressed at the same level in all cell types, but that the sequence is expressed in a wide range of cell types even though variation in abundance is often observed.

The expression constructs as described above may be present in a vector. The vector may comprise border sequences which permit the transfer and integration of the expression cassette into the genome of the organism or host. The construct may be a plant binary vector, for example a binary transformation vector based on pPZP (Hajdukiewicz, et al. 1994). Other example constructs include pBin19 (see Frisch, D. A., L. W. Harris-Haller, et al. 1995, Plant Molecular Biology 27:405-409).

The constructs of the present disclosure may be introduced into plant cells using Ti plasmids, Ri plasmids, plant virus vectors, direct DNA transformation, micro-injection, electroporation, etc. For reviews of such techniques see for example Weissbach and Weissbach, Methods for Plant Molecular Biology, Academy Press, New York VIII, pp. 421-463 (1988); Geierson and Corey, Plant Molecular Biology, 2d Ed. (1988); and Miki and Iyer, Fundamentals of Gene Transfer in Plants. In Plant Metabolism, 2d Ed. DT. Dennis, DH Turpin, DD Lefebvre, D B Layzell (eds), Addison Wesly, Langmans Ltd. London, pp. 561-579 (1997). Other methods include direct DNA uptake, the use of liposomes, electroporation, for example using protoplasts, micro-injection, microprojectiles or whiskers, and vacuum infiltration. See, for example, Bilang, et al. (1991, Gene 100:247-250), Scheid et al. (1991, Mol. Gen. Genet. 228:104-112), Guerche et al. (1987, Plant Science 52:111-116), Neuhause et al. (1987, Theor. Appl Genet. 75:30-36), Klein et al. (2987, Nature 327:70-73); Freeman et al. (1984, Plant Cell Physiol. 29:1353), Howell et al. (1985, Science 227:1229-1231), DeBlock et al. (1989, Plant Physiology 91:694-701), Methods for Plant Molecular Biology (Weissbach and Weissbach, eds., Academic Press Inc., 1988), Methods in Plant Molecular Biology (Schuler and Zielinski, eds., Academic Press Inc., 1989), WO 92/09696, WO 94/00583, EP 331083, EP 175966, Liu and Lomonossoff (2002, J Virol Meth, 105:343-348), EP 290395; WO 8706614; U.S. Pat. Nos. 4,945,050; 5,036,006; and 5,100,792, U.S. patent application Ser. No. 08/438,666, filed May 10, 1995, and Ser. No. 07/951,715, filed Sep. 25, 1992, (all of which are hereby incorporated by reference).

Transient expression methods may be used to express the constructs of the present disclosure (see D'Aoust et al., 2009, Methods in molecular biology, Vol 483, pages 41-50; Liu and Lomonossoff, 2002, Journal of Virological Methods, 105:343-348; which is incorporated herein by reference). Alternatively, a vacuum-based transient expression method, as described by Kapila et al. (1997, Plant Sci. 122, 101-108; which is incorporated herein by reference), or WO 00/063400, WO 00/037663 (which are incorporated herein by reference) may be used. These methods may include, for example, but are not limited to, a method of Agro-inoculation or Agro-infiltration, syringe infiltration, however, other transient methods may also be used as noted above. With Agro-inoculation, Agro-infiltration, or syringe infiltration, a mixture of Agrobacteria comprising the desired nucleic acid enter the intercellular spaces of a tissue, for example the leaves, aerial portion of the plant (including stem, leaves and flower), other portion of the plant (stem, root, flower), or the whole plant. After crossing the epidermis, the Agrobacteria infect and transfer t-DNA copies into the cells. The t-DNA is episomally transcribed and the mRNA translated, leading to the production of the protein of interest in infected cells, however, the passage of t-DNA inside the nucleus is transient.

Also considered part of this disclosure are transgenic plants, plant cells or seeds containing the gene construct of the present disclosure that may be used as a platform plant suitable for transient protein expression described herein. Methods of regenerating whole plants from plant cells are also known in the art (for example see Guerineau and Mullineaux (1993, Plant transformation and expression vectors. In: Plant Molecular Biology Labfax (Croy RRD ed) Oxford, BIOS Scientific Publishers, pp 121-148). In general, transformed plant cells are cultured in an appropriate medium, which may contain selective agents such as antibiotics, where selectable markers are used to facilitate identification of transformed plant cells. Once callus forms, shoot formation can be encouraged by employing the appropriate plant hormones in accordance with known methods and the shoots transferred to rooting medium for regeneration of plants. The plants may then be used to establish repetitive generations, either from seeds or using vegetative propagation techniques. Transgenic plants can also be generated without using tissue culture. Methods for stable transformation, and regeneration of these organisms are established in the art and known to one of skill in the art. Available techniques are reviewed in Vasil et al. (Cell Culture and Somatic Cell Genetics of Plants, Vol I, II and III, Laboratory Procedures and Their Applications, Academic Press, 1984), and Weissbach and Weissbach (Methods for Plant Molecular Biology, Academic Press, 1989). The method of obtaining transformed and regenerated plants is not critical to the present invention.

If plants, plant portions or plant cells are to be transformed or co-transformed by two or more nucleic acid constructs, the nucleic acid construct may be introduced into the Agrobacterium in a single transfection event so that the nucleic acids are pooled, and the bacterial cells transfected. Alternatively, the constructs may be introduced serially. In this case, a first construct is introduced into the Agrobacterium as described, the cells are grown under selective conditions (e.g. in the presence of an antibiotic) where only the singly transformed bacteria can grow. Following this first selection step, a second nucleic acid construct is introduced into the Agrobacterium as described, and the cells are grown under double-selective conditions, where only the double-transformed bacteria can grow. The double-transformed bacteria may then be used to transform a plant, portion of the plant or plant cell as described herein, or may be subjected to a further transformation step to accommodate a third nucleic acid construct.

Alternatively, if plants, plant portions, or plant cells are to be transformed or co-transformed by two or more nucleic acid constructs, the nucleic acid construct may be introduced into the plant by co-infiltrating a mixture of Agrobacterium cells with the plant, plant portion, or plant cell, each Agrobacterium cell may comprise one or more constructs to be introduced within the plant. In order to vary the relative expression levels within the plant, plant portion or plant cell, of a nucleotide sequence of interest within a construct, during the step of infiltration, the concentration of the various Agrobacteria populations comprising the desired constructs may be varied.

TABLE 3
SEQ ID NOs and Description of Sequences

| SEQ ID NO: | Description of Sequence |
|---|---|
| SEQ ID NO: 1 | wt HA B/Washington/02/2019 AA |
| SEQ ID NO: 2 | wt HA B/Singapore/INFKK-16-0569/2016 AA |
| SEQ ID NO: 3 | wt HA B/Rhode Island/01/2019 AA |
| SEQ ID NO: 4 | wt HA B/Michigan/01/2021 AA |
| SEQ ID NO: 5 | wt HA B/Henan-Xigong/1118/2021 AA |
| SEQ ID NO: 6 | wt HA B/Austria/1359417/2021 AA |
| SEQ ID NO: 7 | wt HA B/Singapore/WUH4618/2021 AA |
| SEQ ID NO: 8 | IF-SpPDI.c DNA |
| SEQ ID NO: 9 | IF-H1cTMCT.s1-4r DNA |
| SEQ ID NO: 10 | B/Singapore/INFKK-16-0569/2016 (PrL−) DNA |
| SEQ ID NO: 11 | B/Singapore/INFKK-16-0569/2016 (PrL−) AA |
| SEQ ID NO: 12 | B/Singapore/INFKK-16-0569/2016 (PrL−) + |
| | L404I DNA |
| SEQ ID NO: 13 | B/Singapore/INFKK-16-0569/2016 (PrL−) + |
| | L404I AA |
| SEQ ID NO: 14 | B/Washington/02/2019 (PrL−) DNA |
| SEQ ID NO: 15 | B/Washington/02/2019 (PrL−) AA |
| SEQ ID NO: 16 | B/Washington/02/2019 (PrL−) + L402I DNA |
| SEQ ID NO: 17 | B/Washington/02/2019 (PrL−) + L402I AA |
| SEQ ID NO: 18 | B/Rhode Island/01/2019 (PrL−) DNA |
| SEQ ID NO: 19 | B/Rhode Island/01/2019 (PrL−) AA |
| SEQ ID NO: 20 | B/Rhode Island/01/2019 (PrL−) + L402I DNA |
| SEQ ID NO: 21 | B/Rhode Island/01/2019 (PrL−) + L402I AA |
| SEQ ID NO: 22 | B/Michigan/01/2021 (PrL−) DNA |
| SEQ ID NO: 23 | B/Michigan/01/2021 (PrL−) AA |
| SEQ ID NO: 24 | B/Michigan/01/2021 (PrL−) + L402I DNA |
| SEQ ID NO: 25 | B/Michigan/01/2021 (PrL−) + L402I AA |
| SEQ ID NO: 26 | B/Henan-Xigong/1118/2021 (PrL−) DNA |
| SEQ ID NO: 27 | B/Henan-Xigong/1118/2021 (PrL−) AA |
| SEQ ID NO: 28 | B/Henan-Xigong/1118/2021 (PrL−) + L402I DNA |
| SEQ ID NO: 29 | B/Henan-Xigong/1118/2021 (PrL−) + L402I AA |
| SEQ ID NO: 30 | B/Singapore/WUH4618/2021 (PrL−) DNA |
| SEQ ID NO: 31 | B/Singapore/WUH4618/2021 (PrL−) AA |
| SEQ ID NO: 32 | B/Singapore/WUH4618/2021 (PrL−) + L402I DNA |
| SEQ ID NO: 33 | B/Singapore/WUH4618/2021 (PrL−) + L402I AA |
| SEQ ID NO: 34 | B/Austria/1359417/2021 (PrL−) DNA |
| SEQ ID NO: 35 | B/Austria/1359417/2021 (PrL−) AA |
| SEQ ID NO: 36 | B/Austria/1359417/2021 (PrL−) + L402I DNA |
| SEQ ID NO: 37 | B/Austria/1359417/2021 (PrL−) + L402I AA |
| SEQ ID NO: 38 | Cloning vector 4498 from left to right T-DNA |
| SEQ ID NO: 39 | Construct 2879 from 2X35S prom to NOS term DNA |
| SEQ ID NO: 40 | B HA2 consensus sequence |
| SEQ ID NO: 41 | B HA2 fusion peptide and ectodomain consensus |
| | sequence |
| SEQ ID NO: 42 | B HA2 ectodomain consensus sequence |
| SEQ ID NO: 43 | wt H5 A/Indonesia/5/2005 AA |

The present invention will be further illustrated in the following examples.

### Example 1: Influenza HA Constructs

The influenza HA constructs were produced using techniques well known within the art. For example, wildtype B/Singapore/INFKK-16-0569/2016, was cloned as described below. Other modified influenza B HA were obtained using similar techniques and the HA sequences primers, templates and products are described in Example 3 (Influenza HA and VLP Production in Plants) and Table 5.

A summary of the parent (unmodified) and modified HA proteins, primers, templates and products are provided in Table 5 below.

Influenza B HA from Different Strains with M2 in 2X35S-CPMV 160-NOS Term (Constructs Number 2879, 8894, 7679, 8881, 8424, 7787, 9627, 9628, 9629, 9630, 9866, 9867, 9868 and 9869)

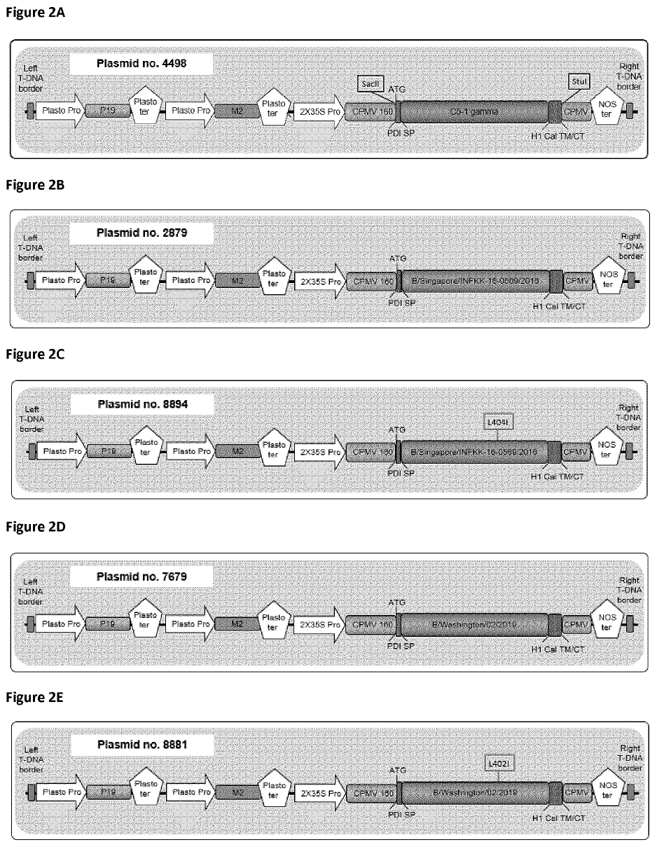

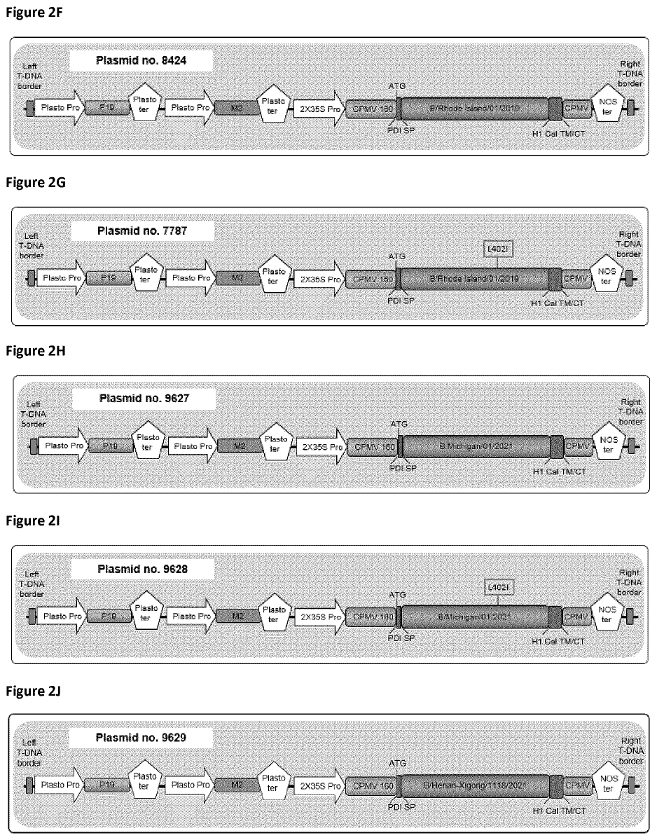

A sequence encoding HA0 from Influenza HA from B/Singapore/INFKK-16-0569/2016 in which the native signal peptide has been replaced by that of alfalfa protein disulfide isomerase (PDISP/HA B/Singapore/INFKK-16-0569/2016) was cloned into 2X35S/CPMV160/NOS expression system (CPMV160) with M2 from Influenza strain A/New/Caledonia/20/1999 using the following PCR-based method. A fragment containing the PDISP/HA B/Singapore/INFKK-16-0569/2016 coding sequence was amplified using primers IF-SpPDI.c (SEQ ID NO: 8) and IF-HcTMCT.s1-4r (SEQ ID NO: 9), using PDISP/HA B/Singapore/INFKK-16-0569/2016 sequence (SEQ ID NO: 10) as template. The PCR product was cloned in 2X35S/CPMV160/NOS expression system using In-Fusion cloning system (Clontech, Mountain View, CA). Construct number 4498 (FIG. 2A) was digested with SacII and StuI restriction enzymes and the linearized plasmids were used for the In-Fusion assembly reaction. Construct number 4498 is an acceptor plasmid intended for “In Fusion” cloning of genes of interest in a 2X35S/CPMV160/NOS-based expression cassette. It also incorporates a gene construct for the co-expression of the TBSV P19 suppressor of silencing under the alfalfa Plastocyanin gene promoter and terminator and for the co-expression of M2 from Influenza strain A/New/Caledonia/20/1999 using the same promoter and terminator. The backbone is a pCAMBIA binary plasmid and the sequence from left to right t-DNA borders is presented in FIG. 2A (SEQ ID NO: 38). The resulting construct was given number 2879 (SEQ ID NO: 39) and a representation of plasmid 2879 is presented in FIG. 2B. The amino acid sequence of mature HA0 from Influenza HA from B/Singapore/INFKK-16-0569/2016 fused with PDISP is presented the sequence of SEQ ID NO: 11). The introduction of modifications into the B HA protein is described in Example 3.

### Example 2: Methods

Agrobacterium tumefaciens Transfection

Agrobacterium tumefaciens strain AGL1 was transfected by electroporation with the parent (unmodified) influenza HA or mutant influenza HA expression vectors using the methods described by D'Aoust et al., 2008 (Plant Biotech. J. 6:930-40). Transfected Agrobacterium were grown in YEB medium supplemented with 10 mM 2-(N-morpholino) ethanesulfonic acid (MES), 20 μM acetosyringone, 50 μg/ml kanamycin and 25 μg/ml of carbenicillin pH5.6 to an OD₆₀₀ between 0.6 and 1.6. Agrobacterium suspensions were centrifuged before use and resuspended in infiltration medium (10 mM MgCl₂ and 10 mM MES pH 5.6).

### Preparation of Plant Biomass, Inoculum and Agroinfiltration

N. benthamiana plants were grown from seeds in flats filled with a commercial peat moss substrate. The plants were allowed to grow in the greenhouse under a 16/8 photoperiod and a temperature regime of 25° C. day/20° C. night. Three weeks after seeding, individual plantlets were picked out, transplanted in pots and left to grow in the greenhouse for three additional weeks under the same environmental conditions.

Agrobacteria transfected with each parent influenza HA or mutant influenza HA expression vector were grown in a YEB medium supplemented with 10 mM 2-(N-morpholino) ethanesulfonic acid (MES), 20 μM acetosyringone, 50 μg/ml kanamycin and 25 μg/ml of carbenicillin pH 5.6 until they reached an OD₆₀₀ between 0.6 and 1.6. Agrobacterium suspensions were centrifuged before use and resuspended in infiltration medium (10 mM MgCl₂ and 10 mM MES pH 5.6) and stored overnight at 4° C. On the day of infiltration, culture batches were diluted in 2.5 culture volumes and allowed to warm before use. Whole plants of N. benthamiana were placed upside down in the bacterial suspension in an air-tight stainless-steel tank under a vacuum of 20-40 Torr for 2-min. Plants were returned to the greenhouse for a 6- or 9-day incubation period until harvest.

### Leaf Harvest and Total Protein Extraction

Proteins were extracted from fresh biomass cut into ~1 cm² pieces by an overnight enzymatic extraction at room temperature using an orbital shaker. The slurry was then filtered through a large pore nylon filter to remove coarse undigested vegetal tissue.

In planta yields were assessed on clarified crude extracts and analyzed using a capillary-based electrophoresis method (Protein Simple, BioTechne) technology and a WES analysis system. In brief, soluble proteins from crude extracts were separated by molecular weight in a capillary and fixed to the matrix. Anti-HA antibody (Novus biological, cat #NB100-56578) is used for detection according to the manufacturer instructions. Yield fold-change is measured to evaluate the change in HA protein. In planta yields fold-change for modified HA normalized to the appropriate parent HA are depicted in FIG. 3.

Drug substance (DS) yield fold change was assessed after small-scale clarification and purification to remove the impurities by densitometry analysis of coomassie-stained protein on SDS gel and immunologically relevant products are included in quantification and purity measurement. Drug substance (DS) yield fold change (%) are shown for mutant HA normalized to the appropriate parent HA in FIG. 4, as further described in the present application.

### Example 3: Modified Influenza B HA and VLP Production in Plants

### A. Modification of B HA

The modified influenza B HA constructs were produced using techniques well known within the art (see Example 1). A summary of the parent (unmodified) and modified HA proteins, primers, templates and products is provided in Table 5 below. The sequences used are provided in Example 5 and in the sequence listing.

### B/Singapore/INFKK-16-0569/2016

L404I B/Singapore/INFKK-16-0569/2016 Mutant HA was constructed by mutating the leucine residue at position 404 of parent B/Singapore/INFKK-16-0569/2016 to isoleucine (Construct #8894). As shown in FIG. 3, purified extracts from N. benthamiana plants agroinfiltrated with Construct #8894 exhibited an approximate 2.3 fold increase in plant yield as compared to extracts from N. benthamiana plants agroinfiltrated with parent B/Singapore/INFKK-16-0569/2016 (Construct #2879).

### B/Washington/09/19

L402I B/Washington/09/19 Mutant HA was constructed by mutating the leucine residue at position 402 of parent B/Washington/09/19 HA to isoleucine (Construct #8881). As shown in FIG. 3, purified extracts from N. benthamiana plants agroinfiltrated with Construct #8881 exhibited an approximate 1.9 fold increase in in planta yield as compared to extracts from N. benthamiana plants agroinfiltrated with parent B/Washington/09/19 (Construct #7679).

### B/Rhode Island/01/2019

L402I B/Rhode Island/01/2019 Mutant HA was constructed by mutating the leucine residue at position 402 of parent B/Rhode Island/01/2019 to isoleucine (Construct #7787). As shown in FIG. 3, purified extracts from N. benthamiana plants agroinfiltrated with Construct #7787 exhibited an approximate 1.5 fold increase in in planta yield as compared to extracts from N. benthamiana plants agroinfiltrated with parent B/Rhode Island/01/2019 HA (Construct #8424). B/Michigan/01/2021

L402I B/Michigan/01/2021 Mutant HA was constructed by mutating the leucine residue at position 402 of parent B/Michigan/01/2021 HA to isoleucine (Construct #9628). As shown in FIG. 3, purified extracts from N. benthamiana plants agroinfiltrated with Construct #9628 exhibited an approximate 1.5 fold increase in in planta yield as compared to extracts from N. benthamiana plants agroinfiltrated with parent B/Michigan/01/2021 HA (Construct #9627).

### B/Henan-Xigong/1118/2021

L402I B/Henan-Xigong/1118/2021 Mutant HA was constructed by mutating the leucine residue at position 402 of parent B/Henan-Xigong/1118/2021 HA to isoleucine (Construct #9630). As shown in FIG. 3, purified extracts from N. benthamiana plants agroinfiltrated with Construct #9630 exhibited an approximate 1.2 fold increase in in plant yield as compared to extracts from N. benthamiana plants agroinfiltrated with parent B/Henan-Xigong/1118/2021 HA (Construct #9629).

### B/Singapore/WUH4618/2021

L402I B/Singapore/WUH4618/2021 Mutant HA was constructed by mutating the leucine residue at position 402 of parent B/Singapore/WUH4618/2021 HA to isoleucine (Construct #9867). As shown in FIG. 3, purified extracts from N. benthamiana plants agroinfiltrated with Construct #9867 exhibited an approximate 1.3 fold increase in in planta yield as compared to extracts from N. benthamiana plants agroinfiltrated with parent B/Singapore/WUH4618/2021 (Construct #9866).

### B/Austria/1359417/2021

L402I B/Austria/1359417/2021 Mutant HA was constructed by mutating the leucine residue at position 402 of parent B/Austria/1359417/2021 HA to isoleucine (Construct #9869). As shown in FIG. 3, purified extracts from N. benthamiana plants agroinfiltrated with Construct #9869 exhibited an approximate 1.3 fold increase in in planta yield as compared to extracts from N. benthamiana plants agroinfiltrated with parent B/Austria/1359417/2021 HA (Construct #9868).

The one or more than one modification described herein specifically increase influenza HA protein production and VLP yield in plants.

### Example 4: In Planta Yield and Drug Substance (DS) Yield

A summary of the in planta and drug substance (DS) yield is given in Table 4. In planta yield were measured as described in Example 2. The in planta yield fold-changes were obtained by comparing the yield of the mutated or modified HA protein to the appropriate parent HA (see FIG. 3).

The DS yield fold-changes were obtained by comparing the yield of the mutated or modified HA protein to the appropriate unmodified (parent) HA (see FIG. 4).

TABLE 4
In-planta and drug substance (DS) yield increase of modified
B HA protein (L402I) compared to unmodified B HA protein

| | In planta | Drug Substance |
|---|---|---|
| Modified B HA | Yield Change | Yield Change |
| B/Singapore/INFKK-16-0569/2016 | 2.3 | |
| B/Washington/09/19 | 1.9 | 2.6 |
| B/Rhode Island/01/2019 | 1.5 | 1.8 |
| B/Michigan/01/2021 | 1.5 | |
| B/Henan-Xigong/1118/2021 | 1.2 | |
| B/Singapore/WUH4618/2021 | 1.3 | |
| B/Austria/1359417/2021 | 1.3 | |

TABLE 5
Examples of constructs that have been prepared as described herein.
Sequences are provided in Example 5 and the sequence listing.

| | | Primer 1 | Primer 2 | Template for |
|---|---|---|---|---|
| Gene of interest (GOI) | Construct | (forward) | (reverse) | PCR |
| B/Singapore/INFKK-16-0569/2016 (PrL−) | 2879 | SEQ ID NO: 8 | SEQ ID NO: 9 | SEQ ID NO: 10 |
| B/Singapore/INFKK-16-0569/2016 (PrL−) + L404I | 8894 | SEQ ID NO: 8 | SEQ ID NO: 9 | SEQ ID NO: 12 |
| B/Washington/02/2019 (PrL−) | 7679 | SEQ ID NO: 8 | SEQ ID NO: 9 | SEQ ID NO: 14 |
| B/Washington/02/2019 (PrL−) + L402I | 8881 | SEQ ID NO: 8 | SEQ ID NO: 9 | SEQ ID NO: 16 |
| B/Rhode Island/01/2019 (PrL−) | 8424 | SEQ ID NO: 8 | SEQ ID NO: 9 | SEQ ID NO: 18 |
| B/Rhode Island/01/2019 (PrL−) + L402I | 7787 | SEQ ID NO: 8 | SEQ ID NO: 9 | SEQ ID NO: 20 |
| B/Michigan/01/2021 (PrL−) | 9627 | SEQ ID NO: 8 | SEQ ID NO: 9 | SEQ ID NO: 22 |
| B/Michigan/01/2021 (PrL−) + L402I | 9628 | SEQ ID NO: 8 | SEQ ID NO: 9 | SEQ ID NO: 24 |
| B/Henan-Xigong/1118/2021 (PrL−) | 9629 | SEQ ID NO: 8 | SEQ ID NO: 9 | SEQ ID NO: 26 |
| B/Henan-Xigong/1118/2021 (PrL−) + L402I | 9630 | SEQ ID NO: 8 | SEQ ID NO: 9 | SEQ ID NO: 28 |
| B/Singapore/WUH4618/2021 (PrL−) | 9866 | SEQ ID NO: 8 | SEQ ID NO: 9 | SEQ ID NO: 30 |
| B/Singapore/WUH4618/2021 (PrL−) + L402I | 9867 | SEQ ID NO: 8 | SEQ ID NO: 9 | SEQ ID NO: 32 |
| B/Austria/1359417/2021 (PrL−) | 9868 | SEQ ID NO: 8 | SEQ ID NO: 9 | SEQ ID NO: 34 |
| B/Austria/1359417/2021 (PrL−) + L402I | 9869 | SEQ ID NO: 8 | SEQ ID NO: 9 | SEQ ID NO: 36 |

| | Resulting | Resulting | Acceptor |
|---|---|---|---|
| Gene of interest (GOI) | Gene | Protein | Plasmid |
| B/Singapore/INFKK-16-0569/2016 (PrL−) | SEQ ID NO: 10 | SEQ ID NO: 11 | 4498 SacII-StuI |
| B/Singapore/INFKK-16-0569/2016 (PrL−) + L404I | SEQ ID NO: 12 | SEQ ID NO: 13 | 4498 SacII-StuI |
| B/Washington/02/2019 (PrL−) | SEQ ID NO: 14 | SEQ ID NO: 15 | 4498 SacII-StuI |
| B/Washington/02/2019 (PrL−) + L402I | SEQ ID NO: 16 | SEQ ID NO: 17 | 4498 SacII-StuI |
| B/Rhode Island/01/2019 (PrL−) | SEQ ID NO: 18 | SEQ ID NO: 19 | 4498 SacII-StuI |
| B/Rhode Island/01/2019 (PrL−) + L402I | SEQ ID NO: 20 | SEQ ID NO: 21 | 4498 SacII-StuI |
| B/Michigan/01/2021 (PrL−) | SEQ ID NO: 22 | SEQ ID NO: 23 | 4498 SacII-StuI |
| B/Michigan/01/2021 (PrL−) + L402I | SEQ ID NO: 24 | SEQ ID NO: 25 | 4498 SacII-StuI |
| B/Henan-Xigong/1118/2021 (PrL−) | SEQ ID NO: 26 | SEQ ID NO: 27 | 4498 SacII-StuI |
| B/Henan-Xigong/1118/2021 (PrL−) + L402I | SEQ ID NO: 28 | SEQ ID NO: 29 | 4498 SacII-StuI |
| B/Singapore/WUH4618/2021 (PrL−) | SEQ ID NO: 30 | SEQ ID NO: 31 | 4498 SacII-StuI |
| B/Singapore/WUH4618/2021 (PrL−) + L402I | SEQ ID NO: 32 | SEQ ID NO: 33 | 4498 SacII-StuI |
| B/Austria/1359417/2021 (PrL−) | SEQ ID NO: 34 | SEQ ID NO: 35 | 4498 SacII-StuI |
| B/Austria/1359417/2021 (PrL−) + L402I | SEQ ID NO: 36 | SEQ ID NO: 37 | 4498 SacII-StuI |

### Example 5: Sequences

The following sequences were used (also see Table 4):

wt HA B/Washington/02/2019 AA
(SEQ ID NO: 1)
DRICTGITSSNSPHVVKTATQGEVNVTGVIPLTTTPTKSHFANLKGTETRGKLCPKCLNCTDLDVALGR
PKCTGKIPSARVSILHEVRPVTSGCFPIMHDRTKIRQLPNLLRGYEHVRLSTHNVINAEDAPGRPYEIG
TSGSCPNITNGNGFFATMAWAVPKNKTATNPLTIEVPYICTEGEDQITVWGFHSDNETQMAKLYGDSKP
QKFTSSANGVTTHYVSQIGGFPNQTEDGGLPQSGRIVVDYMVQKSGKTGTITYQRGILLPQKVWCASGR
SKVIKGSLPLIGEADCLHEKYGGLNKSKPYYTGEHAKAIGNCPIWVKTPLKLANGTKYRPPAKLLKERG
FFGAIAGFLEGGWEGMIAGWHGYTSHGAHGVAVAADLKSTQEAINKITKNLNSLSELEVKNLQRLSGAM
DELHNEILELDEKVDDLRADTISSQIELAVLLSNEGIINSEDEHLLALERKLKKMLGPSAVEIGNGCFE
TKHKCNQTCLDRIAAGTEDAGEFSLPTFDSLNITAASLNDDGLDNHTILLYYSTAASSLAVTLMIAIFV
VYMVSRDNVSCSICL
wt HA B/Singapore/INFKK-16-0569/2016 AA
(SEQ ID NO: 2)
DRICTGITSSNSPHVVKTATQGEVNVTGVIPLTTTPTKSYFANLKGTRTRGKLCPDCLNCTDLDVALGR
PMCVGTTPSAKASILHEVRPVTSGCFPIMHDRTKIRQLPNLLRGYEKIRLSTQNVIDAEKAPGGPYRLG
TSGSCPNATSKIGFFATMAWAVPKDNYKNATNPQTVEVPYICTEGEDQITVWGFHSDNKTQMKSLYGDS
NPQKFTSSANGVTTHYVSQIGDFPDQTEDGGLPQSGRIVVDYMVQKPGKTGTIVYQRGVLLPQKVWCAS
GRSKVIKGSLPLIGEADCLHEEYGGLNKSKPYYTGKHAKAIGNCPIWVKTPLKLANGTKYRPPAKLLKE
RGFFGAIAGFLEGGWEGMIAGWHGYTSHGAHGVAVAADLKSTQEAINKITKNLNSLSELEVKNLQRLSG
AMDELHNEILELDEKVDDLRADTISSQIELAVLLSNEGIINSEDEHLLALERKLKKMLGPSAVDIGNGC
FETKHKCNQTCLDRIAAGTENAGEFSLPTFDSLNITAASLNDDGLDNHTILLYYSTAASSLAVTLMLAI
FIVYMVSRDNVSCSICL
wt HA B/Rhode Island/01/2019 AA
(SEQ ID NO: 3)
DRICTGITSSNSPHVVKTATQGEVNVTGVIPLTTTPTKSHFANLKGTETRGKLCPKCLNCTDLDVALGR
PKCTGKIPSARVSILHEVRPVTSGCFPIMHDRTKIRQLPNLLRGYEHVRLSTHNVINAEDAPGGPYEIG
TSGSCPNITNGKGFFATMAWAVPKNKTATNPLTIEVPYICTEEEDQITVWGFHSDDETQMAKLYGDSKP
QKFTSSANGVTTHYVSQIGGFPNQTEDGGLPQSGRIVVDYMVQKSGKTGTITYQRGILLPQKVWCASGK
SKVIKGSLPLIGEADCLHEKYGGLNKSKPYYTGEHAKAIGNCPIWVKTPLKLANGTKYRPPAKLLKERG
FFGAIAGFLEGGWEGMIAGWHGYTSHGAHGVAVAADLKSTQEAINKITKNLNSLSELEVKNLQRLSGAM
DELHNEILELDEKVDDLRADTISSQIELAVLLSNEGIINSEDEHLLALERKLKKMLGPSAVEIGNGCFE
TKHKCNQTCLDRIAAGTEDAGEFSLPTFDSLNITAASLNDDGLDNHTILLYYSTAASSLAVTLMIAIFV
VYMVSRDNVSCSICL
wt HA B/Michigan/01/2021 AA
(SEQ ID NO: 4)
DRICTGITSSNSPHVVKTATQGEVNVTGVIPLTTTPTKSHFANLKGTETRGKLCPKCLNCTDLDVALGR
PKCTGKIPSARVSILHEVRPVTSGCFPIMHDRTKIRQLPNLLRGYEHVRLSTHNVINTEDAPGGPYEIG
TSGSCLNITNGKGFFATMAWAVPKNKTATNPLTIEVPYICAEEEDQITVWGFHSDDETQMARLYGDSKP
QKFTSSANGVTTHYVSQIGGFPNQTEDGGLPQSGRIVVDYMVQKSGKTGTITYQRGILLPQKVWCASGK
SKVIKGSLPLIGEADCLHEKYGGLNKSKPYYTGEHAKAIGNCPIWVKTPLKLANGTKYRPPAKLLKERG
FFGAIAGFLEGGWEGMIAGWHGYTSHGAHGVAVAADLKSTQEAINKITKNLNSLSELEVKNLQRLSGAM
DELHNEILELDEKVDDLRADTISSQIELAVLLSNEGIINSEDEHLLALERKLKKMLGPSAVEIGNGCFE
TKHKCNQTCLDRIAAGTFDAGEFSLPTFDSLNITAASLNDDGLDNHTILLYYSTAASSLAVTLMIAIFV
VYMVSRDNVSCSICL
wt HA B/Henan-Xigong/1118/2021 AA
(SEQ ID NO: 5)
DRICTGITSSNSPHVVKTATQGEVNVTGVIPLTTTPTKSHFANLKGTETRGKLCPKCLNCTDLDVALGR
PKCTGKIPSARVSILHEVRPVTSGCFPIMHDRTKIRQLPNLLRGYEHVRLSTQNVINTEDAPGGPYEIG
TSGSCLNITNGKGFFATMAWAVPKNKTATNPLTIEVPYICTEEEDQITVWGFHSDDETQMARLYGDSKP
QKFTSSANGVTTHYVSQIGGFPNQTEDGGLPQSGRIVVDYMVQKSGKTGTITYQRGILLPQKVWCASGK
SKVIKGSLPLIGEADCLHEKYGGLNKSKPYYTGEHAKAIGNCPIWVKTPLKLANGTKYRPPAKLLKERG
FFGAIAGFLEGGWEGMIAGWHGYTSHGAHGVAVAADLKSTQEAINKITKNLNSLSELEVKNLQRLSGAM
DELHNEILELDEKVDDLRADTISSQIELAVLLSNEGIINSEDEHLLALERKLKKMLGPSAVEIGNGCFE
TKHKCNQTCLDRIAAGTEDAGEFSLPTFDSLNITAASLNDDGLDNHTILLYYSTAASSLAVTLMIAIFV
VYMVSRDNVSCSICL
wt HA B/Austria/1359417/2021 AA
(SEQ ID NO: 6)
DRICTGITSSNSPHVVKTATQGEVNVTGVIPLTTTPTKSHFANLKGTETRGKLCPKCLNCTDLDVALGR
PKCTGKIPSARVSILHEVRPVTSGCFPIMHDRTKIRQLPNLLRGYEHVRLSTHNVINTEDAPGGPYEIG
TSGSCLNITNGKGFFATMAWAVPKNKTATNPLTIEVPYICTEEEDQITVWGFHSDDETQMARLYGDSKP
QKFTSSANGVTTHYVSQIGGFPNQTEDGGLPQSGRIVVDYMVOKSGKTGTITYQRGILLPQKVWCASGK
SKVIKGSLPLIGEADCLHEKYGGLNKSKPYYTGEHAKAIGNCPIWVKTPLKLANGTKYRPPAKLLKERG
FFGAIAGFLEGGWEGMIAGWHGYTSHGAHGVAVAADLKSTQEAINKITKNLNSLSELEVKNLQRLSGAM
DELHNEILELDEKVDDLRADTISSQIELAVLLSNEGIINSEDEHLLALERKLKKMLGPSAVEIGNGCFE
TKHKCNQTCLDRIAAGTEDAGEFSLPTFDSLNITAASLNDDGLDNHTILLYYSTAASSLAVTLMIAIFV
VYMVSRDNVSCSICL
wt HA B/Singapore/WUH4618/2021 AA
(SEQ ID NO: 7)
DRICTGITSSNSPHVVKTATQGEVNVTGVIPLTTTPTKSHFANLKGTETRGKLCPKCLNCTDLDVALGR
PKCTGKIPSARVSILHEVRPVTSGCFPIMHDRTKIRQLPNLLRGYEHVRLSTHNVINTEDAPGGPYEIG
TSGSCLNITNGKGFFATMAWAVPKNKTATNPLTIEVPYICTEEEDQITVWGFHSDDETQMVRLYGDSKP
QKFTSSANGVTTHYVSQIGGFPNQTEDGGLPQSGRIVVDYMVQKSGKTGTITYQRGILLPQKVWCASGK
SKVIKGSLPLIGEADCLHEKYGGLNKSKPYYTGEHAKAIGNCPIWVKTPLKLANGTKYRPPAKLLKERG
FFGAIAGFLEGGWEGMIAGWHGYTSHGAHGVAVAADLKSTQEAINKITKNLNSLSELEVKNLQRLSGAM
DELHNEILELDEKVDDLRADTISSQIELAVLLSNEGIINSEDEHLLALERKLKKMLGPSAVEIGNGCFE
TKHKCNQTCLDRIAAGTFDAGEFSLPTFDSLNITAASLNDDGLDNHTILLYYSTAASSLAVTLMIAIFV
VYMVSRDNVSCSICL
IF-SpPDI.c DNA
(SEQ ID NO: 8)
TCGTGCTTCGGCACCAGTACAATGGCGAAAAACGTTGCGATTTTCGGCTTATTGTTTTCTCTTCTTGTG
IF-H1cTMCT.s1-4r DNA
(SEQ ID NO: 9)
ACTAAAGAAAATAGGCCTTTAAATACATATTCTACACTGTAGAGAC
B/Singapore/INFKK-16-0569/2016 (PrL-) DNA
(SEQ ID NO: 10)
atggcgaaaaacgttgcgattttcggcttattgttttctcttcttgtgttggttccttctcagatcttc
gcggatcgaatctgcactgggataacatcttcaaactcacctcatgtggtcaaaacagctactcaaggg
gaggtcaatgtgactggcgtgataccactgacaacaacaccaacaaaatcttattttgcaaatctcaaa
ggaacaaggaccagagggaaactatgcccggactgtctcaactgtacagatctggatgtggccttgggc
aggccaatgtgtgtggggaccacaccttctgctaaagcttcaatactccatgaggtcagacctgttaca
tccgggtgctttcctataatgcacgacagaacaaaaatcaggcaactacccaatcttctcagaggatat
gaaaagatcaggttatcaacccaaaacgttatcgatgcagaaaaagcaccaggaggaccctacagactt
ggaacctcaggatcttgccctaacgctaccagtaaaatcggattttttgcaacaatggcttgggctgtc
ccaaaggacaactacaaaaatgcaacgaacccacaaacagtggaagtaccatacatttgtacagaaggg
gaagaccaaattactgtttgggggttccattcggataacaaaacccaaatgaagagcctctatggagac
tcaaatcctcaaaagttcacctcatctgctaatggagtaaccacgcattatgtttctcagattggcgac
ttcccagatcaaacagaagacggaggactaccacaaagcggcagaattgttgttgattacatggtgcaa
aaacctgggaaaacaggaacaattgtctatcaaaggggtgttttgttgcctcaaaaggtgtggtgcgcg
agtggcaggagcaaagtaataaaagggtcattgcctttaattggtgaagcagattgccttcatgaagaa
tacggtggattaaacaaaagcaagccttactacacaggaaaacatgcaaaagccataggaaattgccca
atatgggtaaaaacacctttgaagcttgccaatggaaccaaatatagacctcctggtggaggatgggaa
ggaatgattgcaggttggcacggatacacatctcacggagcacatggagtggcagtggcggcagacctt
aagagtacacaagaagctataaataagataacaaaaaatctcaattctttgagtgaactagaagtaaag
aaccttcaaagactaagtggtgccatggatgaactccacaacgaaatactcgagctggatgaaaaagtg
gatgatctcagagctgacactataagctcacaaatagaacttgcagtcttgctttccaacgaaggaata
ataaacagtgaagacgagcatctattggcacttgagagaaaactaaagaaaatgctgggtccctctgct
gtagacataggaaacggatgcttcgaaaccaaacacaaatgcaaccagacctgcttagacaggatagct
gctggcacctttaatgcaggagaattttctctccccacttttgattcattgaacattactgctgcatct
ttaaatgatgatggattggataactaccagattttggcgatctattcaactgtcgccagttcattggta
ctggtagtctccctgggggcaatcagtttctggatgtgctctaatgggtctctacagtgtagaatatgt
atttaa
B/Singapore/INFKK-16-0569/2016 (PrL-) AA
(SEQ ID NO: 11)
MAKNVAIFGLLESLLVLVPSQIFADRICTGITSSNSPHVVKTATQGEVNVTGVIPLTTTPTKSYFANLK
GTRTRGKLCPDCLNCTDLDVALGRPMCVGTTPSAKASILHEVRPVTSGCFPIMHDRTKIRQLPNLLRGY
EKIRLSTQNVIDAEKAPGGPYRLGTSGSCPNATSKIGFFATMAWAVPKDNYKNATNPQTVEVPYICTEG
EDQITVWGFHSDNKTQMKSLYGDSNPQKFTSSANGVTTHYVSQIGDEPDQTEDGGLPQSGRIVVDYMVQ
KPGKTGTIVYQRGVLLPQKVWCASGRSKVIKGSLPLIGEADCLHEEYGGLNKSKPYYTGKHAKAIGNCP
IWVKTPLKLANGTKYRPPGGGWEGMIAGWHGYTSHGAHGVAVAADLKSTQEAINKITKNLNSLSELEVK
NLQRLSGAMDELHNEILELDEKVDDLRADTISSQIELAVLLSNEGIINSEDEHLLALERKLKKMLGPSA
VDIGNGCFETKHKCNQTCLDRIAAGTFNAGEFSLPTFDSLNITAASLNDDGLDNYQILAIYSTVASSLV
LVVSLGAISFWMCSNGSLQCRICI*
B/Singapore/INFKK-16-0569/2016 (PrL-) + L404I DNA
(SEQ ID NO: 12)
atggcgaaaaacgttgcgattttcggcttattgttttctcttcttgtgttggttccttctcagatcttc
gcggatcgaatctgcactgggataacatcttcaaactcacctcatgtggtcaaaacagctactcaaggg
gaggtcaatgtgactggcgtgataccactgacaacaacaccaacaaaatcttattttqcaaatctcaaa
ggaacaaggaccagagggaaactatgcccggactgtctcaactgtacagatctggatgtggccttgggc
aggccaatgtgtgtggggaccacaccttctgctaaagcttcaatactccatgaggtcagacctgttaca
tccgggtgctttcctataatgcacgacagaacaaaaatcaggcaactacccaatcttctcagaggatat
gaaaagatcaggttatcaacccaaaacgttatcgatgcagaaaaagcaccaggaggaccctacagactt
ggaacctcaggatcttgccctaacgctaccagtaaaatcggattttttgcaacaatggcttgggctgtc
ccaaaggacaactacaaaaatgcaacgaacccacaaacagtggaagtaccatacatttgtacagaaggg
gaagaccaaattactgtttgggggttccattcggataacaaaacccaaatgaagagcctctatggagac
tcaaatcctcaaaagttcacctcatctgctaatggagtaaccacgcattatgtttctcagattggcgac
ttcccagatcaaacagaagacggaggactaccacaaagcggcagaattgttgttgattacatggtgcaa
aaacctgggaaaacaggaacaattgtctatcaaaggggtgttttgttgcctcaaaaggtgtggtgcgcg
agtggcaggagcaaagtaataaaagggtcattgcctttaattggtgaagcagattgccttcatgaagaa
tacggtggattaaacaaaagcaagccttactacacaggaaaacatgcaaaagccataggaaattgccca
atatgggtaaaaacacctttgaagcttgccaatggaaccaaatatagacctcctggtggaggatgggaa
ggaatgattgcaggttggcacggatacacatctcacggagcacatggagtggcagtggcggcagacctt
aagagtacacaagaagctataaataagataacaaaaaatctcaattctttgagtgaaatcgaagtaaag
aaccttcaaagactaagtggtgccatggatgaactccacaacgaaatactcgagctggatgaaaaagtg
gatgatctcagagctgacactataagctcacaaatagaacttgcagtcttgctttccaacgaaggaata
ataaacagtgaagacgagcatctattggcacttgagagaaaactaaagaaaatgctgggtccctctgct
gtagacataggaaacggatgcttcgaaaccaaacacaaatgcaaccagacctgcttagacaggatagct
gctggcacctttaatgcaggagaattttctctccccacttttgattcattgaacattactgctgcatct
ttaaatgatgatggattggataactaccagattttggcgatctattcaactgtcgccagttcattggta
ctggtagtctccctgggggcaatcagtttctggatgtgctctaatgggtctctacagtgtagaatatgt
atttaa
B/Singapore/INFKK-16-0569/2016 (PrL-) + L404I AA
(SEQ ID NO: 13)
MAKNVAIFGLLFSLLVLVPSQIFADRICTGITSSNSPHVVKTATQGEVNVTGVIPLTTTPTKSYFANLK
GTRTRGKLCPDCLNCTDLDVALGRPMCVGTTPSAKASILHEVRPVTSGCFPIMHDRTKIRQLPNLLRGY
EKIRLSTQNVIDAEKAPGGPYRLGTSGSCPNATSKIGFFATMAWAVPKDNYKNATNPQTVEVPYICTEG
EDQITVWGFHSDNKTQMKSLYGDSNPQKFTSSANGVTTHYVSQIGDFPDQTEDGGLPQSGRIVVDYMVQ
KPGKTGTIVYQRGVLLPQKVWCASGRSKVIKGSLPLIGEADCLHEEYGGLNKSKPYYTGKHAKAIGNCP
IWVKTPLKLANGTKYRPPGGGWEGMIAGWHGYTSHGAHGVAVAADLKSTQEAINKITKNLNSLSEIEVK
NLQRLSGAMDELHNEILELDEKVDDLRADTISSQIELAVLLSNEGIINSEDEHLLALERKLKKMLGPSA
VDIGNGCFETKHKCNQTCLDRIAAGTENAGEFSLPTFDSLNITAASLNDDGLDNYQILAIYSTVASSLV
LVVSLGAIS FWMCSNGSLQCRICI*
B/Washington/02/2019 (PrL-) DNA
(SEQ ID NO: 14)
atggcgaaaaacgttgcgattttcggcttattgttttctcttcttgtgttggttccttctcagatcttc
gcggatcgaatctgcactgggataacatcgtcaaactcaccacatgtcgtcaaaactgctactcaaggg
gaggtcaatgtgaccggtgtaataccactgacaacaacacccaccaaatctcattttgcaaatctcaaa
ggaacagaaaccagggggaaactatgcccaaaatgcctcaactgcacagatctggatgtagccttgggc
agaccaaaatgcacagggaaaataccctctgcaagggtttcaatactccatgaagtcagacctgttaca
tctgggtgctttcctataatgcacgatagaacaaaaattagacagctgcctaaccttctccgaggatac
gaacatgtcaggttatcaactcacaacgttatcaatgcagaagatgcaccaggaagaccctacgagatt
ggaacctcagggtcttgccctaacattaccaatggaaacggattcttcgcaacaatggcttgggccgtc
ccaaaaaaaaaacagcaacaaatccattaacaatagaagtaccatacatttgtacagaaggagaagac
caaattaccgtttgggggttccactctgacaacgagacccaaatggcaaagctctatggggactcaaag
ccccagaagttcacctcatctgccaacggagtgaccacacattacgtttcacagattggtggcttccca
aatcaaacagaagacggaggactaccacaaagtggcagaattgttgttgattacatggtgcaaaaatct
ggaaaaacaggaacaattacctatcaaagaggtattttattgcctcaaaaggtgtggtgcgcaagtggc
aggagcaaggtaataaaaggatccttgcctttaattggagaagcagattgcctccatgaaaaatacggt
ggattaaacaaaagcaagccttactacacaggggaacatgcaaaggccataggaaattgcccaatatgg
gtgaaaacacccttgaagctggccaatggaaccaaatatagacctcctggtggaggatgggaaggaatg
attgcaggttggcacggatacacatcccatggggcacatggagtagcggtggcagcagaccttaagagc
actcaagaggccataaacaagataacaaaaaatctcaactctttgagtgagctggaagtaaagaatctt
caaagactaagcggtgccatggatgaactccacaacgaaatactagaactagatgagaaagtggatgat
ctcagagctgatacaataagctcacaaatagaactcgcagtcctgctttccaatgaaggaataataaac
agtgaagatgaacatctcttggcgcttgaaagaaagctgaagaaaatgctgggcccctctgctgtagag
atagggaatggatgctttgaaaccaaacacaagtgcaaccagacctgtctcgacagaatagctgctggt
acctttgatgcaggagaattttctctccccacctttgattcactgaatattactgctgcatctttaaat
gacgatggattggataattaccagattttggcgatctattcaactgtcgccagttcattggtactggta
gtctccctgggggcaatcagtttctggatgtgctctaatgggtctctacagtgtagaatatgtatttaa
B/Washington/02/2019 (PrL-) AA
(SEQ ID NO: 15)
MAKNVAIFGLLFSLLVLVPSQIFADRICTGITSSNSPHVVKTATQGEVNVTGVIPLTTTPTKSHFANLK
GTETRGKLCPKCLNCTDLDVALGRPKCTGKIPSARVSILHEVRPVTSGCFPIMHDRTKIRQLPNLLRGY
EHVRLSTHNVINAEDAPGRPYEIGTSGSCPNITNGNGFFATMAWAVPKNKTATNPLTIEVPYICTEGED
QITVWGFHSDNETQMAKLYGDSKPQKFTSSANGVTTHYVSQIGGFPNQTEDGGLPQSGRIVVDYMVQKS
GKTGTITYQRGILLPQKVWCASGRSKVIKGSLPLIGEADCLHEKYGGLNKSKPYYTGEHAKAIGNCPIW
VKTPLKLANGTKYRPPGGGWEGMIAGWHGYTSHGAHGVAVAADLKSTQEAINKITKNLNSLSELEVKNL
QRLSGAMDELHNEILELDEKVDDLRADTISSQIELAVLLSNEGIINSEDEHLLALERKLKKMLGPSAVE
IGNGCFETKHKCNQTCLDRIAAGTEDAGEFSLPTFDSLNITAASLNDDGLDNYQILAIYSTVASSLVLV
VSLGAISFWMCSNGSLQCRICI*
B/Washington/02/2019 (PrL-) + L402I DNA
(SEQ ID NO: 16)
atggcgaaaaacgttgcgattttcggcttattgttttctcttcttgtgttggttccttctcagatcttc
gcggatcgaatctgcactgggataacatcgtcaaactcaccacatgtcgtcaaaactgctactcaaggg
gaggtcaatgtgaccggtgtaataccactgacaacaacacccaccaaatctcattttgcaaatctcaaa
ggaacagaaaccagggggaaactatgcccaaaatgcctcaactgcacagatctggatgtagccttgggc
agaccaaaatgcacagggaaaataccctctgcaagggtttcaatactccatgaagtcagacctgttaca
tctgggtgctttcctataatgcacgatagaacaaaaattagacagctgcctaaccttctccgaggatac
gaacatgtcaggttatcaactcacaacgttatcaatgcagaagatgcaccaggaagaccctacgagatt
ggaacctcagggtcttgccctaacattaccaatggaaacggattcttcgcaacaatggcttgggccgtc
ccaaaaaacaaaacagcaacaaatccattaacaatagaagtaccatacatttgtacagaaggagaagac
caaattaccgtttgggggttccactctgacaacgagacccaaatggcaaagctctatggggactcaaag
ccccagaagttcacctcatctgccaacggagtgaccacacattacgtttcacagattggtggcttccca
aatcaaacagaagacggaggactaccacaaagtggcagaattgttgttgattacatggtgcaaaaatct
ggaaaaacaggaacaattacctatcaaagaggtattttattgcctcaaaaggtgtggtgcgcaagtggc
aggagcaaggtaataaaaggatccttgcctttaattggagaagcagattgcctccatgaaaaatacggt
ggattaaacaaaagcaagccttactacacaggggaacatgcaaaggccataggaaattgcccaatatgg
gtgaaaacacccttgaagctggccaatggaaccaaatatagacctcctggtggaggatgggaaggaatg
attgcaggttggcacggatacacatcccatggggcacatggagtagcggtggcagcagaccttaagagc
actcaagaggccataaacaagataacaaaaaatctcaactctttgagtgagatcgaagtaaagaatctt
caaagactaagcggtgccatggatgaactccacaacgaaatactagaactagatgagaaagtggatgat
ctcagagctgatacaataagctcacaaatagaactcgcagtcctgctttccaatgaaggaataataaac
agtgaagatgaacatctcttggcgcttgaaagaaagctgaagaaaatgctgggcccctctgctgtagag
atagggaatggatgctttgaaaccaaacacaagtgcaaccagacctgtctcgacagaatagctgctggt
acctttgatgcaggagaattttctctccccacctttgattcactgaatattactgctgcatctttaaat
gacgatggattggataattaccagattttggcgatctattcaactgtcgccagttcattggtactggta
gtctccctgggggcaatcagtttctggatgtgctctaatgggtctctacagtgtagaatatgtatttaa
B/Washington/02/2019 (PrL-) + L402I AA
(SEQ ID NO: 17)
MAKNVAIFGLLFSLLVLVPSQIFADRICTGITSSNSPHVVKTATQGEVNVTGVIPLTTTPTKSHFANLK
GTETRGKLCPKCLNCTDLDVALGRPKCTGKIPSARVSILHEVRPVTSGCFPIMHDRTKIRQLPNLLRGY
EHVRLSTHNVINAEDAPGRPYEIGTSGSCPNITNGNGEFATMAWAVPKNKTATNPLTIEVPYICTEGED
QITVWGFHSDNETQMAKLYGDSKPQKFTSSANGVTTHYVSQIGGFPNQTEDGGLPQSGRIVVDYMVQKS
GKTGTITYQRGILLPQKVWCASGRSKVIKGSLPLIGEADCLHEKYGGLNKSKPYYTGEHAKAIGNCPIW
VKTPLKLANGTKYRPPGGGWEGMIAGWHGYTSHGAHGVAVAADLKSTQEAINKITKNLNSLSEIEVKNL
QRLSGAMDELHNEILELDEKVDDLRADTISSQIELAVLLSNEGIINSEDEHLLALERKLKKMLGPSAVE
IGNGCFETKHKCNQTCLDRIAAGTEDAGEFSLPTFDSLNITAASLNDDGLDNYQILAIYSTVASSLVLV
VSLGAISFWMCSNGSLQCRICI*
B/Rhode Island/01/2019 (PrL-) DNA
(SEQ ID NO: 18)
atggcgaaaaacgttgcgattttcggcttattgttttctcttcttgtgttggttccttctcagatcttc
gcggatcgaatctgcactgggataacatcgtcaaactcaccacatgtcgtcaaaactgctactcaaggg
gaggtcaatgtgaccggtgtaataccactgacaacaacacccaccaaatctcattttgcaaatctcaaa
ggaacagaaaccagggggaaactatgcccaaaatgcctcaactgcacagatctggatgtagccttgggc
agaccaaaatgcacagggaaaataccctctgcaagggtttcaatactccatgaagtcagacctgttaca
tctgggtgctttcctataatgcacgatagaacaaaaattagacagctgcctaaccttctccgaggatac
gaacatgtcaggttatcaactcacaacgttatcaatgcagaagatgcaccaggaggaccctacgagatt
ggaacctcagggtcttgccctaacattaccaatggaaagggattcttcgcaacaatggcttgggccgtc
ccaaaaaacaaaacagcaacaaatccattaacaatagaagtaccatacatttgtacagaagaggaagac
caaattaccgtttgggggttccactctgacgacgagacccaaatggcaaagctctatggggactcaaag
ccccagaagttcacctcatctgccaacggagtgaccacacattacgtttcacagattggtggcttccca
aatcaaacagaagacggaggactaccacaaagtggcagaattgttgttgattacatggtgcaaaaatct
ggaaaaacaggaacaattacctatcaaagaggtattttattgcctcaaaaggtgtggtgcgcaagtggc
aagagcaaggtaataaaaggatccttgcctttaattggagaagcagattgcctccatgaaaaatacggt
ggattaaacaaaagcaagccttactacacaggggaacatgcaaaggccataggaaattgcccaatatgg
gtgaaaacacccttgaagctggccaatggaaccaaatatagacctcctggtggaggatgggaaggaatg
attgcaggttggcacggatacacatcccatggggcacatggagtagcggtggcagcagaccttaagagc
actcaagaggccataaacaagataacaaaaaatctcaactctttgagtgagctggaagtaaagaatctt
caaagactaagcggtgccatggatgaactccacaacgaaatactagaactagatgagaaagtggatgat
ctcagagctgatacaataagctcacaaatagaactcgcagtcctgctttccaatgaaggaataataaac
agtgaagatgaacatctcttggcgcttgaaagaaagctgaagaaaatgctgggcccctctgctgtagag
atagggaatggatgctttgaaaccaaacacaagtgcaaccagacctgtctcgacagaatagctgctggt
acctttgatgcaggagaattttctctccccacctttgattcactgaatattactgctgcatctttaaat
gacgatggattggataattaccagattttggcgatctattcaactgtcgccagttcattggtactggta
gtctccctgggggcaatcagtttctggatgtgctctaatgggtctctacagtgtagaatatgtatttaa
B/Rhode Island/01/2019 (PrL-) AA
(SEQ ID NO: 19)
MAKNVAIFGLLFSLLVLVPSQIFADRICTGITSSNSPHVVKTATQGEVNVTGVIPLTTTPTKSHFANLK
GTETRGKLCPKCLNCTDLDVALGRPKCTGKIPSARVSILHEVRPVTSGCFPIMHDRTKIRQLPNLLRGY
EHVRLSTHNVINAEDAPGGPYEIGTSGSCPNITNGKGFFATMAWAVPKNKTATNPLTIEVPYICTEEED
QITVWGFHSDDETQMAKLYGDSKPQKFTSSANGVTTHYVSQIGGFPNQTEDGGLPQSGRIVVDYMVQKS
GKTGTITYQRGILLPQKVWCASGKSKVIKGSLPLIGEADCLHEKYGGLNKSKPYYTGEHAKAIGNCPIW
VKTPLKLANGTKYRPPGGGWEGMIAGWHGYTSHGAHGVAVAADLKSTQEAINKITKNLNSLSELEVKNL
QRLSGAMDELHNEILELDEKVDDLRADTISSQIELAVLLSNEGIINSEDEHLLALERKLKKMLGPSAVE
IGNGCFETKHKCNQTCLDRIAAGTFDAGEFSLPTFDSLNITAASLNDDGLDNYQILAIYSTVASSLVLV
VSLGAISFWMCSNGSLQCRICI*
B/Rhode Island/01/2019 (PrL-) + L402I DNA
(SEQ ID NO: 20)
atggcgaaaaacgttgcgattttcggcttattgttttctcttcttgtgttggttccttctcagatcttc
gcggatcgaatctgcactgggataacatcgtcaaactcaccacatgtcgtcaaaactgctactcaaggg
gaggtcaatgtgaccggtgtaataccactgacaacaacacccaccaaatctcattttgcaaatctcaaa
ggaacagaaaccagggggaaactatgcccaaaatgcctcaactgcacagatctggatgtagccttgggc
agaccaaaatgcacagggaaaataccctctgcaagggtttcaatactccatgaagtcagacctgttaca
tctgggtgctttcctataatgcacgatagaacaaaaattagacagctgcctaaccttctccgaggatac
gaacatgtcaggttatcaactcacaacgttatcaatgcagaagatgcaccaggaggaccctacgagatt
ggaacctcagggtcttgccctaacattaccaatggaaagggattcttcgcaacaatggcttgggccgtc
ccaaaaaacaaaacagcaacaaatccattaacaatagaagtaccatacatttgtacagaagaggaagac
caaattaccgtttgggggttccactctgacgacgagacccaaatggcaaagctctatggggactcaaag
ccccagaagttcacctcatctgccaacggagtgaccacacattacgtttcacagattggtggcttccca
aatcaaacagaagacggaggactaccacaaagtggcagaattgttgttgattacatggtgcaaaaatct
ggaaaaacaggaacaattacctatcaaagaggtattttattgcctcaaaaggtgtggtgcgcaagtggc
aagagcaaggtaataaaaggatccttgcctttaattggagaagcagattgcctccatgaaaaatacggt
ggattaaacaaaagcaagccttactacacaggggaacatgcaaaggccataggaaattgcccaatatgg
gtgaaaacacccttgaagctggccaatggaaccaaatatagacctcctggtggaggatgggaaggaatg
attgcaggttggcacggatacacatcccatggggcacatggagtagcggtggcagcagaccttaagagc
actcaagaggccataaacaagataacaaaaaatctcaactctttgagtgagATCgaagtaaagaatctt
caaagactaagcggtgccatggatgaactccacaacgaaatactagaactagatgagaaagtggatgat
ctcagagctgatacaataagctcacaaatagaactcgcagtcctgctttccaatgaaggaataataaac
agtgaagatgaacatctcttggcgcttgaaagaaagctgaagaaaatgctgggcccctctgctgtagag
atagggaatggatgctttgaaaccaaacacaagtgcaaccagacctgtctcgacagaatagctgctggt
acctttgatgcaggagaattttctctccccacctttgattcactgaatattactgctgcatctttaaat
gacgatggattggataattaccagattttggcgatctattcaactgtcgccagttcattggtactggta
gtctccctgggggcaatcagtttctggatgtgctctaatgggtctctacagtgtagaatatgtatttaa
B/Rhode Island/01/2019 (PrL-) + L4021 AA
(SEQ ID NO: 21)
MAKNVAIFGLLFSLLVLVPSQIFADRICTGITSSNSPHVVKTATQGEVNVTGVIPLTTTPTKSHFANLK
GTETRGKLCPKCLNCTDLDVALGRPKCTGKIPSARVSILHEVRPVTSGCFPIMHDRTKIRQLPNLLRGY
EHVRLSTHNVINAEDAPGGPYEIGTSGSCPNITNGKGFFATMAWAVPKNKTATNPLTIEVPYICTEEED
QITVWGFHSDDETQMAKLYGDSKPQKFTSSANGVTTHYVSQIGGFPNQTEDGGLPQSGRIVVDYMVQKS
GKTGTITYQRGILLPQKVWCASGKSKVIKGSLPLIGEADCLHEKYGGLNKSKPYYTGEHAKAIGNCPIW
VKTPLKLANGTKYRPPGGGWEGMIAGWHGYTSHGAHGVAVAADLKSTQEAINKITKNLNSLSEIEVKNL
QRLSGAMDELHNEILELDEKVDDLRADTISSQIELAVLLSNEGIINSEDEHLLALERKLKKMLGPSAVE
IGNGCFETKHKCNQTCLDRIAAGTEDAGEFSLPTFDSLNITAASLNDDGLDNYQILAIYSTVASSLVLV
VSLGAISFWMCSNGSLQCRICI*
B/Michigan/01/2021 (PrL-) DNA
(SEQ ID NO: 22)
atggcgaaaaacgttgcgattttcggcttattgttttctcttcttgtgttggttccttctcagatcttc
gcggatcgaatctgcactgggataacatcgtcaaactcaccacatgtcgtcaaaactgctactcaaggg
gaggtcaatgtgaccggtgtaataccactgacaacaacacccaccaaatctcattttgcaaatctcaaa
ggaacagaaaccagggggaaactatgcccaaaatgcctcaactgcacagatctggatgtagccttgggc
agaccaaaatgcacagggaaaataccctctgcaagggtttcaatactccatgaagtcagacctgttaca
tctgggtgctttcctataatgcacgatagaacaaaaattagacagctgcctaaccttctccgaggatac
gaacatgtcaggttatcaactcacaacgttatcaatACCgaagatgcaccaggaggaccctacgagatt
ggaacctcagggtcttgcCTGaacattaccaatggaaagggattcttcgcaacaatggcttgggccgtc
ccaaaaaacaaaacagcaacaaatccattaacaatagaagtaccatacatttgtGCCgaagaggaagac
caaattaccgtttgggggttccactctgacGACgagacccaaatggcaAGActctatggggactcaaag
ccccagaagttcacctcatctgccaacggagtgACCacacattacgtttcacagattggtggcttccca
aatcaaacagaagacggaggactaccacaaagtggcagaattgttgttgattacatggtgcaaaaatct
ggaaaaacaggaacaattacctatcaaagaggtattttattgcctcaaaaggtgtggtgcgcaagtggc
aagagcaaggtaataaaaggatccttgcctttaattggagaagcagattgcctccatgaaaaatacggt
ggattaaacaaaagcaagccttactacacaggggaacatgcaaaggccataggaaattgcccaatatgg
gtgaaaacacccttgaagctggccaatggaaccaaatatagacctcctggtggaggatgggaaggaatg
attgcaggttggcacggatacacatcccatggggcacatggagtagcggtggcagcagaccttaagagc
actcaagaggccataaacaagataacaaaaaatctcaactctttgagtgagctggaagtaaagaatctt
caaagactaagcggtgccatggatgaactccacaacgaaatactagaactagatgagaaagtggatgat
ctcagagctgatacaataagctcacaaatagaactcgcagtcctgctttccaatgaaggaataataaac
agtgaagatgaacatctcttggcgcttgaaagaaagctgaagaaaatgctgggcccctctgctgtagag
atagggaatggatgctttgaaaccaaacacaagtgcaaccagacctgtctcgacagaatagctgctggt
acctttgatgcaggagaattttctctccccacctttgattcactgaatattactgctgcatctttaaat
gacgatggattggataattaccagattttggcgatctattcaactgtcgccagttcattggtactggta
gtctccctgggggcaatcagtttctggatgtgctctaatgggtctctacagtgtagaatatgtatttaa
B/Michigan/01/2021 (PrL-) AA
(SEQ ID NO: 23)
MAKNVAIFGLLFSLLVLVPSQIFADRICTGITSSNSPHVVKTATQGEVNVTGVIPLTTTPTKSHFANLK
GTETRGKLCPKCLNCTDLDVALGRPKCTGKIPSARVSILHEVRPVTSGCFPIMHDRTKIRQLPNLLRGY
EHVRLSTHNVINTEDAPGGPYEIGTSGSCLNITNGKGFFATMAWAVPKNKTATNPLTIEVPYICAEEED
QITVWGFHSDDETQMARLYGDSKPQKFTSSANGVTTHYVSQIGGFPNQTEDGGLPQSGRIVVDYMVQKS
GKTGTITYQRGILLPQKVWCASGKSKVIKGSLPLIGEADCLHEKYGGLNKSKPYYTGEHAKAIGNCPIW
VKTPLKLANGTKYRPPGGGWEGMIAGWHGYTSHGAHGVAVAADLKSTQEAINKITKNLNSLSELEVKNL
QRLSGAMDELHNEILELDEKVDDLRADTISSQIELAVLLSNEGIINSEDEHLLALERKLKKMLGPSAVE
IGNGCFETKHKCNQTCLDRIAAGTFDAGEFSLPTFDSLNITAASLNDDGLDNYQILAIYSTVASSLVLV
VSLGAISFWMCSNGSLQCRICI*
B/Michigan/01/2021 (PrL-) + L402I DNA
(SEQ ID NO: 24)
atggcgaaaaacgttgcgattttcggcttattgttttctcttcttgtgttggttccttctcagatcttc
gcggatcgaatctgcactgggataacatcgtcaaactcaccacatgtcgtcaaaactgctactcaaggg
gaggtcaatgtgaccggtgtaataccactgacaacaacacccaccaaatctcattttgcaaatctcaaa
ggaacagaaaccagggggaaactatgcccaaaatgcctcaactgcacagatctggatgtagccttgggc
agaccaaaatgcacagggaaaataccctctgcaagggtttcaatactccatgaagtcagacctgttaca
tctgggtgctttcctataatgcacgatagaacaaaaattagacagctgcctaaccttctccgaggatac
gaacatgtcaggttatcaactcacaacgttatcaatACCgaagatgcaccaggaggaccctacgagatt
ggaacctcagggtcttgcCTGaacattaccaatggaaagggattcttcgcaacaatggcttgggccgtc
ccaaaaaacaaaacagcaacaaatccattaacaatagaagtaccatacatttgtGCCgaagaggaagac
caaattaccgtttgggggttccactctgacGACgagacccaaatggcaAGActctatggggactcaaag
ccccagaagttcacctcatctgccaacggagtgACCacacattacgtttcacagattggtggcttccca
aatcaaacagaagacggaggactaccacaaagtggcagaattgttgttgattacatggtgcaaaaatct
ggaaaaacaggaacaattacctatcaaagaggtattttattgcctcaaaaggtgtggtgcgcaagtggc
aagagcaaggtaataaaaggatccttgcctttaattggagaagcagattgcctccatgaaaaatacggt
ggattaaacaaaagcaagccttactacacaggggaacatgcaaaggccataggaaattgcccaatatgg
gtgaaaacacccttgaagctggccaatggaaccaaatatagacctcctggtggaggatgggaaggaatg
attgcaggttggcacggatacacatcccatggggcacatggagtagcggtggcagcagaccttaagagc
actcaagaggccataaacaagataacaaaaaatctcaactctttgagtgagATCgaagtaaagaatctt
caaagactaagcggtgccatggatgaactccacaacgaaatactagaactagatgagaaagtggatgat
ctcagagctgatacaataagctcacaaatagaactcgcagtcctgctttccaatgaaggaataataaac
agtgaagatgaacatctcttggcgcttgaaagaaagctgaagaaaatgctgggcccctctgctgtagag
atagggaatggatgctttgaaaccaaacacaagtgcaaccagacctgtctcgacagaatagctgctggt
acctttgatgcaggagaattttctctccccacctttgattcactgaatattactgctgcatctttaaat
gacgatggattggataattaccagattttggcgatctattcaactgtcgccagttcattggtactggta
gtctccctgggggcaatcagtttctggatgtgctctaatgggtctctacagtgtagaatatgtatttaa
B/Michigan/01/2021 (PrL-) + L402I AA
(SEQ ID NO: 25)
MAKNVAIFGLLFSLLVLVPSQIFADRICTGITSSNSPHVVKTATQGEVNVTGVIPLTTTPTKSHFANLK
GTETRGKLCPKCLNCTDLDVALGRPKCTGKIPSARVSILHEVRPVTSGCFPIMHDRTKIRQLPNLLRGY
EHVRLSTHNVINTEDAPGGPYEIGTSGSCLNITNGKGFFATMAWAVPKNKTATNPLTIEVPYICAEEED
QITVWGFHSDDETQMARLYGDSKPQKFTSSANGVTTHYVSQIGGFPNQTEDGGLPQSGRIVVDYMVQKS
GKTGTITYQRGILLPQKVWCASGKSKVIKGSLPLIGEADCLHEKYGGLNKSKPYYTGEHAKAIGNCPIW
VKTPLKLANGTKYRPPGGGWEGMIAGWHGYTSHGAHGVAVAADLKSTQEAINKITKNLNSLSEIEVKNL
QRLSGAMDELHNEILELDEKVDDLRADTISSQIELAVLLSNEGIINSEDEHLLALERKLKKMLGPSAVE
IGNGCFETKHKCNQTCLDRIAAGTEDAGEFSLPTFDSLNITAASLNDDGLDNYQILAIYSTVASSLVLV
VSLGAISFWMCSNGSLQCRICI*
B/Henan-Xigong/1118/2021 (PrL-) DNA
(SEQ ID NO: 26)
atggcgaaaaacgttgcgattttcggcttattgttttctcttcttgtgttggttccttctcagatcttc
gcggatcgaatctgcactgggataacatcgtcaaactcaccacatgtcgtcaaaactgctactcaaggg
gaggtcaatgtgaccggtgtaataccactgacaacaacacccaccaaatctcattttgcaaatctcaaa
ggaacagaaaccagggggaaactatgcccaaaatgcctcaactgcacagatctggatgtagccttgggc
agaccaaaatgcacagggaaaataccctctgcaagggtttcaatactccatgaagtcagacctgttaca
tctgggtgctttcctataatgcacgatagaacaaaaattagacagctgcctaaccttctccgaggatac
gaacatgtcaggttatcaactCAGaacgttatcaatACCgaagatgcaccaggaggaccctacgagatt
ggaacctcagggtcttgcCTGaacattaccaatggaaagggattcttcgcaacaatggcttgggccgtc
ccaaaaaacaaaacagcaacaaatccattaacaatagaagtaccatacatttgtacagaagaggaagac
caaattaccgtttgggggttccactctgacgacgagacccaaatggcaAGActctatggggactcaaag
ccccagaagttcacctcatctgccaacggagtgaccacacattacgtttcacagattggtggcttccca
aatcaaacagaagacggaggactaccacaaagtggcagaattgttgttgattacatggtgcaaaaatct
ggaaaaacaggaacaattacctatcaaagaggtattttattgcctcaaaaggtgtggtgcgcaagtggc
aagagcaaggtaataaaaggatccttgcctttaattggagaagcagattgcctccatgaaaaatacggt
ggattaaacaaaagcaagccttactacacaggggaacatgcaaaggccataggaaattgcccaatatgg
gtgaaaacacccttgaagctggccaatggaaccaaatatagacctcctggtggaggatgggaaggaatg
attgcaggttggcacggatacacatcccatggggcacatggagtagcggtggcagcagaccttaagagc
actcaagaggccataaacaagataacaaaaaatctcaactctttgagtgagctggaagtaaagaatctt
caaagactaagcggtgccatggatgaactccacaacgaaatactagaactagatgagaaagtggatgat
ctcagagctgatacaataagctcacaaatagaactcgcagtcctgctttccaatgaaggaataataaac
agtgaagatgaacatctcttggcgcttgaaagaaagctgaagaaaatgctgggcccctctgctgtagag
atagggaatggatgctttgaaaccaaacacaagtgcaaccagacctgtctcgacagaatagctgctggt
acctttgatgcaggagaattttctctccccacctttgattcactgaatattactgctgcatctttaaat
gacgatggattggataattaccagattttggcgatctattcaactgtcgccagttcattggtactggta
gtctccctgggggcaatcagtttctggatgtgctctaatgggtctctacagtgtagaatatgtatttaa
B/Henan-Xigong/1118/2021 (PrL-) AA
(SEQ ID NO: 27)
MAKNVAIFGLLFSLLVLVPSQIFADRICTGITSSNSPHVVKTATQGEVNVTGVIPLTTTPTKSHFANLK
GTETRGKLCPKCLNCTDLDVALGRPKCTGKIPSARVSILHEVRPVTSGCFPIMHDRTKIRQLPNLLRGY
EHVRLSTQNVINTEDAPGGPYEIGTSGSCLNITNGKGFFATMAWAVPKNKTATNPLTIEVPYICTEEED
QITVWGFHSDDETQMARLYGDSKPQKFTSSANGVTTHYVSQIGGFPNQTEDGGLPQSGRIVVDYMVQKS
GKTGTITYQRGILLPQKVWCASGKSKVIKGSLPLIGEADCLHEKYGGLNKSKPYYTGEHAKAIGNCPIW
VKTPLKLANGTKYRPPGGGWEGMIAGWHGYTSHGAHGVAVAADLKSTQEAINKITKNLNSLSELEVKNL
QRLSGAMDELHNEILELDEKVDDLRADTISSQIELAVLLSNEGIINSEDEHLLALERKLKKMLGPSAVE
IGNGCFETKHKCNQTCLDRIAAGTEDAGEFSLPTFDSLNITAASLNDDGLDNYQILAIYSTVASSLVLV
VSLGAISFWMCSNGSLQCRICI*
B/Henan-Xigong/1118/2021 (PrL-) + L402I DNA
(SEQ ID NO: 28)
atggcgaaaaacgttgcgattttcggcttattgttttctcttcttgtgttggttccttctcagatcttc
gcggatcgaatctgcactgggataacatcgtcaaactcaccacatgtcgtcaaaactgctactcaaggg
gaggtcaatgtgaccggtgtaataccactgacaacaacacccaccaaatctcattttgcaaatctcaaa
ggaacagaaaccagggggaaactatgcccaaaatgcctcaactgcacagatctggatgtagccttgggc
agaccaaaatgcacagggaaaataccctctgcaagggtttcaatactccatgaagtcagacctgttaca
tctgggtgctttcctataatgcacgatagaacaaaaattagacagctgcctaaccttctccgaggatac
gaacatgtcaggttatcaactCAGaacgttatcaatACCgaagatgcaccaggaggaccctacgagatt
ggaacctcagggtcttgcCTGaacattaccaatggaaagggattcttcgcaacaatggcttgggccgtc
ccaaaaaacaaaacagcaacaaatccattaacaatagaagtaccatacatttgtacagaagaggaagac
caaattaccgtttgggggttccactctgacgacgagacccaaatggcaAGActctatggggactcaaag
ccccagaagttcacctcatctgccaacggagtgaccacacattacgtttcacagattggtggcttccca
aatcaaacagaagacggaggactaccacaaagtggcagaattgttgttgattacatggtgcaaaaatct
ggaaaaacaggaacaattacctatcaaagaggtattttattgcctcaaaaggtgtggtgcgcaagtggc
aagagcaaggtaataaaaggatccttgcctttaattggagaagcagattgcctccatgaaaaatacggt
ggattaaacaaaagcaagccttactacacaggggaacatgcaaaggccataggaaattgcccaatatgg
gtgaaaacacccttgaagctggccaatggaaccaaatatagacctcctggtggaggatgggaaggaatg
attgcaggttggcacggatacacatcccatggggcacatggagtagcggtggcagcagaccttaagagc
actcaagaggccataaacaagataacaaaaaatctcaactctttgagtgagATCgaagtaaagaatctt
caaagactaagcggtgccatggatgaactccacaacgaaatactagaactagatgagaaagtggatgat
ctcagagctgatacaataagctcacaaatagaactcgcagtcctgctttccaatgaaggaataataaac
agtgaagatgaacatctcttggcgcttgaaagaaagctgaagaaaatgctgggcccctctgctgtagag
atagggaatggatgctttgaaaccaaacacaagtgcaaccagacctgtctcgacagaatagctgctggt
acctttgatgcaggagaattttctctccccacctttgattcactgaatattactgctgcatctttaaat
gacgatggattggataattaccagattttggcgatctattcaactgtcgccagttcattggtactggta
gtctccctgggggcaatcagtttctggatgtgctctaatgggtctctacagtgtagaatatgtatttaa
B/Henan-Xigong/1118/2021 (PrL-) + L402I AA
(SEQ ID NO: 29)
MAKNVAIFGLLFSLLVLVPSQIFADRICTGITSSNSPHVVKTATQGEVNVTGVIPLTTTPTKSHFANLK
GTETRGKLCPKCLNCTDLDVALGRPKCTGKIPSARVSILHEVRPVTSGCFPIMHDRTKIRQLPNLLRGY
EHVRLSTQNVINTEDAPGGPYEIGTSGSCLNITNGKGFFATMAWAVPKNKTATNPLTIEVPYICTEEED
QITVWGFHSDDETQMARLYGDSKPQKFTSSANGVTTHYVSQIGGFPNQTEDGGLPQSGRIVVDYMVQKS
GKTGTITYQRGILLPQKVWCASGKSKVIKGSLPLIGEADCLHEKYGGLNKSKPYYTGEHAKAIGNCPIW
VKTPLKLANGTKYRPPGGGWEGMIAGWHGYTSHGAHGVAVAADLKSTQEAINKITKNLNSLSEIEVKNL
QRLSGAMDELHNEILELDEKVDDLRADTISSQIELAVLLSNEGIINSEDEHLLALERKLKKMLGPSAVE
IGNGCFETKHKCNQTCLDRIAAGTFDAGEFSLPTFDSLNITAASLNDDGLDNYQILAIYSTVASSLVLV
VSLGAISFWMCSNGSLQCRICI*
B/Singapore/WUH4618/2021 (PrL-) DNA
(SEQ ID NO: 30)
atggcgaaaaacgttgcgattttcggcttattgttttctcttcttgtgttggttccttctcagatcttc
gcggatcgaatctgcactgggataacatcgtcaaactcaccacatgtcgtcaaaactgctactcaaggg
gaggtcaatgtgaccggtgtaataccactgacaacaacacccaccaaatctcattttgcaaatctcaaa
ggaacagaaaccagggggaaactatgcccaaaatgcctcaactgcacagatctggatgtagccttgggc
agaccaaaatgcacagggaaaataccctctgcaagggtttcaatactccatgaagtcagacctgttaca
tctgggtgctttcctataatgcacgatagaacaaaaattagacagctgcctaaccttctccgaggatac
gaacatgtcaggttatcaactcacaacgttatcaatACCgaagatgcaccaggaggaccctacgagatt
ggaacctcagggtcttgcCTGaacattaccaatggaaagggattcttcgcaacaatggcttgggccgtc
ccaaaaaacaaaacagcaacaaatccattaacaatagaagtaccatacatttgtACCgaagaggaagac
caaattaccgtttgggggttccactctgacGACgagacccaaatgGTGAGActctatggggactcaaag
ccccagaagttcacctcatctgccaacggagtgACCacacattacgtttcacagattggtggcttccca
aatcaaacagaagacggaggactaccacaaagtggcagaattgttgttgattacatggtgcaaaaatct
ggaaaaacaggaacaattacctatcaaagaggtattttattgcctcaaaaggtgtggtgcgcaagtggc
aagagcaaggtaataaaaggatccttgcctttaattggagaagcagattgcctccatgaaaaatacggt
ggattaaacaaaagcaagccttactacacaggggaacatgcaaaggccataggaaattgcccaatatgg
gtgaaaacacccttgaagctggccaatggaaccaaatatagacctcctggtggaggatgggaaggaatg
attgcaggttggcacggatacacatcccatggggcacatggagtagcggtggcagcagaccttaagagc
actcaagaggccataaacaagataacaaaaaatctcaactctttgagtgagctggaagtaaagaatctt
caaagactaagcggtgccatggatgaactccacaacgaaatactagaactagatgagaaagtggatgat
ctcagagctgatacaataagctcacaaatagaactcgcagtcctgctttccaatgaaggaataataaac
agtgaagatgaacatctcttggcgcttgaaagaaagctgaagaaaatgctgggcccctctgctgtagag
atagggaatggatgctttgaaaccaaacacaagtgcaaccagacctgtctcgacagaatagctgctggt
acctttgatgcaggagaattttctctccccacctttgattcactgaatattactgctgcatctttaaat
gacgatggattggataattaccagattttggcgatctattcaactgtcgccagttcattggtactggta
gtctccctgggggcaatcagtttctggatgtgctctaatgggtctctacagtgtagaatatgtatttaa
B/Singapore/WUH4618/2021 (PrL-) AA
(SEQ ID NO: 31)
MAKNVAIFGLLFSLLVLVPSQIFADRICTGITSSNSPHVVKTATQGEVNVTGVIPLTTTPTKSHFANLK
GTETRGKLCPKCLNCTDLDVALGRPKCTGKIPSARVSILHEVRPVTSGCFPIMHDRTKIRQLPNLLRGY
EHVRLSTHNVINTEDAPGGPYEIGTSGSCLNITNGKGFFATMAWAVPKNKTATNPLTIEVPYICTEEED
QITVWGFHSDDETQMVRLYGDSKPQKFTSSANGVTTHYVSQIGGFPNQTEDGGLPQSGRIVVDYMVQKS
GKTGTITYQRGILLPQKVWCASGKSKVIKGSLPLIGEADCLHEKYGGLNKSKPYYTGEHAKAIGNCPIW
VKTPLKLANGTKYRPPGGGWEGMIAGWHGYTSHGAHGVAVAADLKSTQEAINKITKNLNSLSELEVKNL
QRLSGAMDELHNEILELDEKVDDLRADTISSQIELAVLLSNEGIINSEDEHLLALERKLKKMLGPSAVE
IGNGCFETKHKCNQTCLDRIAAGTFDAGEFSLPTFDSLNITAASLNDDGLDNYQILAIYSTVASSLVLV
VSLGAISFWMCSNGSLQCRICI*
B/Singapore/WUH4618/2021 (PrL-) + L402I DNA
(SEQ ID NO: 32)
atggcgaaaaacgttgcgattttcggcttattgttttctcttcttgtgttggttccttctcagatcttc
gcggatcgaatctgcactgggataacatcgtcaaactcaccacatgtcgtcaaaactgctactcaaggg
gaggtcaatgtgaccggtgtaataccactgacaacaacacccaccaaatctcattttgcaaatctcaaa
ggaacagaaaccagggggaaactatgcccaaaatgcctcaactgcacagatctggatgtagccttgggc
agaccaaaatgcacagggaaaataccctctgcaagggtttcaatactccatgaagtcagacctgttaca
tctgggtgctttcctataatgcacgatagaacaaaaattagacagctgcctaaccttctccgaggatac
gaacatgtcaggttatcaactcacaacgttatcaatACCgaagatgcaccaggaggaccctacgagatt
ggaacctcagggtcttgcCTGaacattaccaatggaaagggattcttcgcaacaatggcttgggccgtc
ccaaaaaacaaaacagcaacaaatccattaacaatagaagtaccatacatttgtACCgaagaggaagac
caaattaccgtttgggggttccactctgacGACgagacccaaatgGTGAGActctatggggactcaaag
ccccagaagttcacctcatctgccaacggagtgACCacacattacgtttcacagattggtggcttccca
aatcaaacagaagacggaggactaccacaaagtggcagaattgttgttgattacatggtgcaaaaatct
ggaaaaacaggaacaattacctatcaaagaggtattttattgcctcaaaaggtgtggtgcgcaagtggc
aagagcaaggtaataaaaggatccttgcctttaattggagaagcagattgcctccatgaaaaatacggt
ggattaaacaaaagcaagccttactacacaggggaacatgcaaaggccataggaaattgcccaatatgg
gtgaaaacacccttgaagctggccaatggaaccaaatatagacctcctggtggaggatgggaaggaatg
attgcaggttggcacggatacacatcccatggggcacatggagtagcggtggcagcagaccttaagagc
actcaagaggccataaacaagataacaaaaaatctcaactctttgagtgagATCgaagtaaagaatctt
caaagactaagcggtgccatggatgaactccacaacgaaatactagaactagatgagaaagtggatgat
ctcagagctgatacaataagctcacaaatagaactcgcagtcctgctttccaatgaaggaataataaac
agtgaagatgaacatctcttggcgcttgaaagaaagctgaagaaaatgctgggcccctctgctgtagag
atagggaatggatgctttgaaaccaaacacaagtgcaaccagacctgtctcgacagaatagctgctggt
acctttgatgcaggagaattttctctccccacctttgattcactgaatattactgctgcatctttaaat
gacgatggattggataattaccagattttggcgatctattcaactgtcgccagttcattggtactggta
gtctccctgggggcaatcagtttctggatgtgctctaatgggtctctacagtgtagaatatgtatttaa
B/Singapore/WUH4618/2021 (PrL-) + L402I AA
(SEQ ID NO: 33)
MAKNVAIFGLLFSLLVLVPSQIFADRICTGITSSNSPHVVKTATQGEVNVTGVIPLTTTPTKSHFANLK
GTETRGKLCPKCLNCTDLDVALGRPKCTGKIPSARVSILHEVRPVTSGCFPIMHDRTKIRQLPNLLRGY
EHVRLSTHNVINTEDAPGGPYEIGTSGSCLNITNGKGFFATMAWAVPKNKTATNPLTIEVPYICTEEED
QITVWGFHSDDETQMVRLYGDSKPQKFTSSANGVTTHYVSQIGGFPNQTEDGGLPQSGRIVVDYMVQKS
GKTGTITYQRGILLPQKVWCASGKSKVIKGSLPLIGEADCLHEKYGGLNKSKPYYTGEHAKAIGNCPIW
VKTPLKLANGTKYRPPGGGWEGMIAGWHGYTSHGAHGVAVAADLKSTQEAINKITKNLNSLSEIEVKNL
QRLSGAMDELHNEILELDEKVDDLRADTISSQIELAVLLSNEGIINSEDEHLLALERKLKKMLGPSAVE
IGNGCFETKHKCNQTCLDRIAAGTEDAGEFSLPTFDSLNITAASLNDDGLDNYQILAIYSTVASSLVLV
VSLGAISFWMCSNGSLQCRICI*
B/Austria/1359417/2021 (PrL-) DNA
(SEQ ID NO: 34)
atggcgaaaaacgttgcgattttcggcttattgttttctcttcttgtgttggttccttctcagatcttc
gcggatcgaatctgcactgggataacatcgtcaaactcaccacatgtcgtcaaaactgctactcaaggg
gaggtcaatgtgaccggtgtaataccactgacaacaacacccaccaaatctcattttgcaaatctcaaa
ggaacagaaaccagggggaaactatgcccaaaatgcctcaactgcacagatctggatgtagccttgggc
agaccaaaatgcacagggaaaataccctctgcaagggtttcaatactccatgaagtcagacctgttaca
tctgggtgctttcctataatgcacgatagaacaaaaattagacagctgcctaaccttctccgaggatac
gaacatgtcaggttatcaactcacaacgttatcaatACCgaagatgcaccaggaggaccctacgagatt
ggaacctcagggtcttgcCTGaacattaccaatggaaagggattcttcgcaacaatggcttgggccgtc
ccaaaaaacaaaacagcaacaaatccattaacaatagaagtaccatacatttgtACCgaagaggaagac
caaattaccgtttgggggttccactctgacGACgagacccaaatggcaAGActctatggggactcaaag
ccccagaagttcacctcatctgccaacggagtgACCacacattacgtttcacagattggtggcttccca
aatcaaacagaagacggaggactaccacaaagtggcagaattgttgttgattacatggtgcaaaaatct
ggaaaaacaggaacaattacctatcaaagaggtattttattgcctcaaaaggtgtggtgcgcaagtggc
aagagcaaggtaataaaaggatccttgcctttaattggagaagcagattgcctccatgaaaaatacggt
ggattaaacaaaagcaagccttactacacaggggaacatgcaaaggccataggaaattgcccaatatgg
gtgaaaacacccttgaagctggccaatggaaccaaatatagacctcctggtggaggatgggaaggaatg
attgcaggttggcacggatacacatcccatggggcacatggagtagcggtggcagcagaccttaagagc
actcaagaggccataaacaagataacaaaaaatctcaactctttgagtgagctggaagtaaagaatctt
caaagactaagcggtgccatggatgaactccacaacgaaatactagaactagatgagaaagtggatgat
ctcagagctgatacaataagctcacaaatagaactcgcagtcctgctttccaatgaaggaataataaac
agtgaagatgaacatctcttggcgcttgaaagaaagctgaagaaaatgctgggcccctctgctgtagag
atagggaatggatgctttgaaaccaaacacaagtgcaaccagacctgtctcgacagaatagctgctggt
acctttgatgcaggagaattttctctccccacctttgattcactgaatattactgctgcatctttaaat
gacgatggattggataattaccagattttggcgatctattcaactgtcgccagttcattggtactggta
gtctccctgggggcaatcagtttctggatgtgctctaatgggtctctacagtgtagaatatgtatttaa
B/Austria/1359417/2021 (PrL-) AA
(SEQ ID NO: 35)
MAKNVAIFGLLFSLLVLVPSQIFADRICTGITSSNSPHVVKTATQGEVNVTGVIPLTTTPTKSHFANLK
GTETRGKLCPKCLNCTDLDVALGRPKCTGKIPSARVSILHEVRPVTSGCFPIMHDRTKIRQLPNLLRGY
EHVRLSTHNVINTEDAPGGPYEIGTSGSCLNITNGKGFFATMAWAVPKNKTATNPLTIEVPYICTEEED
QITVWGFHSDDETQMARLYGDSKPQKFTSSANGVTTHYVSQIGGFPNQTEDGGLPQSGRIVVDYMVQKS
GKTGTITYQRGILLPQKVWCASGKSKVIKGSLPLIGEADCLHEKYGGLNKSKPYYTGEHAKAIGNCPIW
VKTPLKLANGTKYRPPGGGWEGMIAGWHGYTSHGAHGVAVAADLKSTQEAINKITKNLNSLSELEVKNL
QRLSGAMDELHNEILELDEKVDDLRADTISSQIELAVLLSNEGIINSEDEHLLALERKLKKMLGPSAVE
IGNGCFETKHKCNQTCLDRIAAGTFDAGEFSLPTFDSLNITAASLNDDGLDNYQILAIYSTVASSLVLV
VSLGAISFWMCSNGSLQCRICI*
B/Austria/1359417/2021 (PrL-) + L402I DNA
(SEQ ID NO: 36)
atggcgaaaaacgttgcgattttcggcttattgttttctcttcttgtgttggttccttctcagatcttc
gcggatcgaatctgcactgggataacatcgtcaaactcaccacatgtcgtcaaaactgctactcaaggg
gaggtcaatgtgaccggtgtaataccactgacaacaacacccaccaaatctcattttgcaaatctcaaa
ggaacagaaaccagggggaaactatgcccaaaatgcctcaactgcacagatctggatgtagccttgggc
agaccaaaatgcacagggaaaataccctctgcaagggtttcaatactccatgaagtcagacctgttaca
tctgggtgctttcctataatgcacgatagaacaaaaattagacagctgcctaaccttctccgaggatac
gaacatgtcaggttatcaactcacaacgttatcaatACCgaagatgcaccaggaggaccctacgagatt
ggaacctcagggtcttgcCTGaacattaccaatggaaagggattcttcgcaacaatggcttgggccgtc
ccaaaaaacaaaacagcaacaaatccattaacaatagaagtaccatacatttgtACCgaagaggaagac
caaattaccgtttgggggttccactctgacGACgagacccaaatggcaAGActctatggggactcaaag
ccccagaagttcacctcatctgccaacggagtgACCacacattacgtttcacagattggtggcttccca
aatcaaacagaagacggaggactaccacaaagtggcagaattgttgttgattacatggtgcaaaaatct
ggaaaaacaggaacaattacctatcaaagaggtattttattgcctcaaaaggtgtggtgcgcaagtggc
aagagcaaggtaataaaaggatccttgcctttaattggagaagcagattgcctccatgaaaaatacggt
ggattaaacaaaagcaagccttactacacaggggaacatgcaaaggccataggaaattgcccaatatgg
gtgaaaacacccttgaagctggccaatggaaccaaatatagacctcctggtggaggatgggaaggaatg
attgcaggttggcacggatacacatcccatggggcacatggagtagcggtggcagcagaccttaagagc
actcaagaggccataaacaagataacaaaaaatctcaactctttgagtgagATCgaagtaaagaatctt
caaagactaagcggtgccatggatgaactccacaacgaaatactagaactagatgagaaagtggatgat
ctcagagctgatacaataagctcacaaatagaactcgcagtcctgctttccaatgaaggaataataaac
agtgaagatgaacatctcttggcgcttgaaagaaagctgaagaaaatgctgggcccctctgctgtagag
atagggaatggatgctttgaaaccaaacacaagtgcaaccagacctgtctcgacagaatagctgctggt
acctttgatgcaggagaattttctctccccacctttgattcactgaatattactgctgcatctttaaat
gacgatggattggataattaccagattttggcgatctattcaactgtcgccagttcattggtactggta
gtctccctgggggcaatcagtttctggatgtgctctaatgggtctctacagtgtagaatatgtatttaa
B/Austria/1359417/2021 (PrL-) + L4021 AA
(SEQ ID NO: 37)
MAKNVAIFGLLFSLLVLVPSQIFADRICTGITSSNSPHVVKTATQGEVNVTGVIPLTTTPTKSHFANLK
GTETRGKLCPKCLNCTDLDVALGRPKCTGKIPSARVSILHEVRPVTSGCFPIMHDRTKIRQLPNLLRGY
EHVRLSTHNVINTEDAPGGPYEIGTSGSCLNITNGKGFFATMAWAVPKNKTATNPLTIEVPYICTEEED
QITVWGFHSDDETQMARLYGDSKPQKFTSSANGVTTHYVSQIGGFPNQTEDGGLPQSGRIVVDYMVQKS
GKTGTITYQRGILLPQKVWCASGKSKVIKGSLPLIGEADCLHEKYGGLNKSKPYYTGEHAKAIGNCPIW
VKTPLKLANGTKYRPPGGGWEGMIAGWHGYTSHGAHGVAVAADLKSTQEAINKITKNLNSLSEIEVKNL
QRLSGAMDELHNEILELDEKVDDLRADTISSQIELAVLLSNEGIINSEDEHLLALERKLKKMLGPSAVE
IGNGCFETKHKCNQTCLDRIAAGTEDAGEFSLPTFDSLNITAASLNDDGLDNYQILAIYSTVASSLVLV
VSLGAISFWMCSNGSLQCRICI*
Cloning vector 4498 from left to right T-DNA
(SEQ ID NO: 38)
TGGCAGGATATATTGTGGTGTAAACAAATTGACGCTTAGACAACTTAATAACACATTGCGGACGTTTTT
AATGTACTGAATTAACGCCGAATCCCGGGCTGGTATATTTATATGTTGTCAAATAACTCAAAAACCATA
AAAGTTTAAGTTAGCAAGTGTGTACATTTTTACTTGAACAAAAATATTCACCTACTACTGTTATAAATC
ATTATTAAACATTAGAGTAAAGAAATATGGATGATAAGAACAAGAGTAGTGATATTTTGACAACAATTT
TGTTGCAACATTTGAGAAAATTTTGTTGTTCTCTCTTTTCATTGGTCAAAAACAATAGAGAGAGAAAAA
GGAAGAGGGAGAATAAAAACATAATGTGAGTATGAGAGAGAAAGTTGTACAAAAGTTGTACCAAAATAG
TTGTACAAATATCATTGAGGAATTTGACAAAAGCTACACAAATAAGGGTTAATTGCTGTAAATAAATAA
GGATGACGCATTAGAGAGATGTACCATTAGAGAATTTTTGGCAAGTCATTAAAAAGAAAGAATAAATTA
TTTTTAAAATTAAAAGTTGAGTCATTTGATTAAACATGTGATTATTTAATGAATTGATGAAAGAGTTGG
ATTAAAGTTGTATTAGTAATTAGAATTTGGTGTCAAATTTAATTTGACATTTGATCTTTTCCTATATAT
TGCCCCATAGAGTCAGTTAACTCATTTTTATATTTCATAGATCAAATAAGAGAAATAACGGTATATTAA
TCCCTCCAAAAAAAAAAAACGGTATATTTACTAAAAAATCTAAGCCACGTAGGAGGATAACAGGATCCC
CGTAGGAGGATAACATCCAATCCAACCAATCACAACAATCCTGATGAGATAACCCACTTTAAGCCCACG
CATCTGTGGCACATCTACATTATCTAAATCACACATTCTTCCACACATCTGAGCCACACAAAAACCAAT
CCACATCTTTATCACCCATTCTATAAAAAATCACACTTTGTGAGTCTACACTTTGATTCCCTTCAAACA
CATACAAAGAGAAGAGACTAATTAATTAATTAATCATCTTGAGAGAAAATGGAACGAGCTATACAAGGA
AACGACGCTAGGGAACAAGCTAACAGTGAACGTTGGGATGGAGGATCAGGAGGTACCACTTCTCCCTTC
AAACTTCCTGACGAAAGTCCGAGTTGGACTGAGTGGCGGCTACATAACGATGAGACGAATTCGAATCAA
GATAATCCCCTTGGTTTCAAGGAAAGCTGGGGTTTCGGGAAAGTTGTATTTAAGAGATATCTCAGATAC
GACAGGACGGAAGCTTCACTGCACAGAGTCCTTGGATCTTGGACGGGAGATTCGGTTAACTATGCAGCA
TCTCGATTTTTCGGTTTCGACCAGATCGGATGTACCTATAGTATTCGGTTTCGAGGAGTTAGTATCACC
GTTTCTGGAGGGTCGCGAACTCTTCAGCATCTCTGTGAGATGGCAATTCGGTCTAAGCAAGAACTGCTA
CAGCTTGCCCCAATCGAAGTGGAAAGTAATGTATCAAGAGGATGCCCTGAAGGTACTCAAACCTTCGAA
AAAGAAAGCGAGTAAGTTAAAATGCTTCTTCGTCTCCTATTTATAATATGGTTTGTTATTGTTAATTTT
GTTCTTGTAGAAGAGCTTAATTAATCGTTGTTGTTATGAAATACTATTTGTATGAGATGAACTGGTGTA
ATGTAATTCATTTACATAAGTGGAGTCAGAATCAGAATGTTTCCTCCATAACTAACTAGACATGAAGAC
CTGCCGCGTACAATTGTCTTATATTTGAACAACTAAAATTGAACATCTTTTGCCACAACTTTATAAGTG
GTTAATATAGCTCAAATATATGGTCAAGTTCAATAGATTAATAATGGAAATATCAGTTATCGAAATTCA
TTAACAATCAACTTAACGTTATTAACTACTAATTTTATATCATCCCCTTTGATAAATGATAGTACACCA
ATTAGGAAGGAGCATGCTCGCCTAGGAGATTGTCGTTTCCCGCCTTCAGTTTGCAAGCTGCTCTAGCCG
TGTAGCCAATACGCAAACCGCCTCTCCCCGCGCGTTGGGAATTACTAGCGCGTGTCGACACGCGTGGCG
CGCCCTGGTATATTTATATGTTGTCAAATAACTCAAAAACCATAAAAGTTTAAGTTAGCAAGTGTGTAC
ATTTTTACTTGAACAAAAATATTCACCTACTACTGTTATAAATCATTATTAAACATTAGAGTAAAGAAA
TATGGATGATAAGAACAAGAGTAGTGATATTTTGACAACAATTTTGTTGCAACATTTGAGAAAATTTTG
TTGTTCTCTCTTTTCATTGGTCAAAAACAATAGAGAGAGAAAAAGGAAGAGGGAGAATAAAAACATAAT
GTGAGTATGAGAGAGAAAGTTGTACAAAAGTTGTACCAAAATAGTTGTACAAATATCATTGAGGAATTT
GACAAAAGCTACACAAATAAGGGTTAATTGCTGTAAATAAATAAGGATGACGCATTAGAGAGATGTACC
ATTAGAGAATTTTTGGCAAGTCATTAAAAAGAAAGAATAAATTATTTTTAAAATTAAAAGTTGAGTCAT
TTGATTAAACATGTGATTATTTAATGAATTGATGAAAGAGTTGGATTAAAGTTGTATTAGTAATTAGAA
TTTGGTGTCAAATTTAATTTGACATTTGATCTTTTCCTATATATTGCCCCATAGAGTCAGTTAACTCAT
TTTTATATTTCATAGATCAAATAAGAGAAATAACGGTATATTAATCCCTCCAAAAAAAAAAAACGGTAT
ATTTACTAAAAAATCTAAGCCACGTAGGAGGATAACAGGATCCCCGTAGGAGGATAACATCCAATCCAA
CCAATCACAACAATCCTGATGAGATAACCCACTTTAAGCCCACGCATCTGTGGCACATCTACATTATCT
AAATCACACATTCTTCCACACATCTGAGCCACACAAAAACCAATCCACATCTTTATCACCCATTCTATA
AAAAATCACACTTTGTGAGTCTACACTTTGATTCCCTTCAAACACATACAAAGAGAAGAGACTAATTAA
TTAATTAATCATCTTGAGAGAAAATGAGTCTTCTAACCGAGGTCGAAACGCCTATCAGAAACGAATGGG
GGTGCAGATGCAACGATTCAAGTGATCCTCTTGTTGTTGCCGCAAGTATAATTGGGATTGTGCACCTGA
TATTGTGGATTATTGATCGCCTTTTTTCCAAAAGCATTTATCGTATCTTTAAACACGGTTTAAAAAGAG
GGCCTTCTACGGAAGGAGTACCAGAGTCTATGAGGGAAGAATATCGAGAGGAACAGCAGAATGCTGTGG
ATGCTGACGATGGTCATTTTGTCAGCATAGAGCTGGAGTAAGAGCTCTAAGTTAAAATGCTTCTTCGTC
TCCTATTTATAATATGGTTTGTTATTGTTAATTTTGTTCTTGTAGAAGAGCTTAATTAATCGTTGTTGT
TATGAAATACTATTTGTATGAGATGAACTGGTGTAATGTAATTCATTTACATAAGTGGAGTCAGAATCA
GAATGTTTCCTCCATAACTAACTAGACATGAAGACCTGCCGCGTACAATTGTCTTATATTTGAACAACT
AAAATTGAACATCTTTTGCCACAACTTTATAAGTGGTTAATATAGCTCAAATATATGGTCAAGTTCAAT
AGATTAATAATGGAAATATCAGTTATCGAAATTCATTAACAATCAACTTAACGTTATTAACTACTAATT
TTATATCATCCCCTTTGATAAATGATAGTACACCAATTAGGAAGGAACTAGGAGATTGTCGTTTCCCGC
CTTCAGTTTGCAAGCTGCTCTAGCCGTGTAGCCAATACGCAAACCGCCTCTCCCCGCGCGTTGGGAATT
ACTAGCGCGTGTCGACAAGCTTGCATGCCGGTCAACATGGTGGAGCACGACACACTTGTCTACTCCAAA
AATATCAAAGATACAGTCTCAGAAGACCAAAGGGCAATTGAGACTTTTCAACAAAGGGTAATATCCGGA
AACCTCCTCGGATTCCATTGCCCAGCTATCTGTCACTTTATTGTGAAGATAGTGGAAAAGGAAGGTGGC
TCCTACAAATGCCATCATTGCGATAAAGGAAAGGCCATCGTTGAAGATGCCTCTGCCGACAGTGGTCCC
AAAGATGGACCCCCACCCACGAGGAGCATCGTGGAAAAAGAAGACGTTCCAACCACGTCTTCAAAGCAA
GTGGATTGATGTGATAACATGGTGGAGCACGACACACTTGTCTACTCCAAAAATATCAAAGATACAGTC
TCAGAAGACCAAAGGGCAATTGAGACTTTTCAACAAAGGGTAATATCCGGAAACCTCCTCGGATTCCAT
TGCCCAGCTATCTGTCACTTTATTGTGAAGATAGTGGAAAAGGAAGGTGGCTCCTACAAATGCCATCAT
TGCGATAAAGGAAAGGCCATCGTTGAAGATGCCTCTGCCGACAGTGGTCCCAAAGATGGACCCCCACCC
ACGAGGAGCATCGTGGAAAAAGAAGACGTTCCAACCACGTCTTCAAAGCAAGTGGATTGATGTGATATC
TCCACTGACGTAAGGGATGACGCACAATCCCACTATCCTTCGCAAGACCCTTCCTCTATATAAGGAAGT
TCATTTCATTTGGAGAGGTATTAAAATCTTAATAGGTTTTGATAAAAGCGAACGTGGGGAAACCCGAAC
CAAACCTTCTTCTAAACTCTCTCTCATCTCTCTTAAAGCAAACTTCTCTCTTGTCTTTCTTGCGTGAGC
GATCTTCAACGTTGTCAGATCGTGCTTCGGCACCGCGGATGGCGAAAAACGTTGCGATTTTCGGCTTAT
TGTTTTCTCTTCTTGTGTTGGTTCCTTCTCAGATCTTCGCCTGCAGGCTCCTCAGCCAAAACGACACCC
CCATCTGTCTATCCACTGGCCCCTGGATCTGCTGCCCAAACTAACTCCATGGTGACCCTGGGATGCCTG
GTCAAGGGCTATTTCCCTGAGCCAGTGACAGTGACCTGGAACTCTGGATCCCTGTCCAGCGGTGTGCAC
ACCTTCCCAGCTGTCCTGCAGTCTGACCTCTACACTCTGAGCAGCTCAGTGACTGTCCCCTCCAGCACC
TGGCCCAGCGAGACCGTCACCTGCAACGTTGCCCACCCGGCCAGCAGCACCAAGGTGGACAAGAAAATT
GTGCCCAGGGATTGTGGTTGTAAGCCTTGCATATGTACAGTCCCAGAAGTATCATCTGTCTTCATCTTC
CCCCCAAAGCCCAAGGATGTGCTCACCATTACTCTGACTCCTAAGGTCACGTGTGTTGTGGTAGACATC
AGCAAGGATGATCCCGAGGTCCAGTTCAGCTGGTTTGTAGATGATGTGGAGGTGCACACAGCTCAGACG
CAACCCCGGGAGGAGCAGTTCAACAGCACTTTCCGCTCAGTCAGTGAACTTCCCATCATGCACCAGGAC
TGGCTCAATGGCAAGGAGCGATCGCTCACCATCACCATCACCATCACCATCACCATTAAAGGCCTATTT
TCTTTAGTTTGAATTTACTGTTATTCGGTGTGCATTTCTATGTTTGGTGAGCGGTTTTCTGTGCTCAGA
GTGTGTTTATTTTATGTAATTTAATTTCTTTGTGAGCTCCTGTTTAGCAGGTCGTCCCTTCAGCAAGGA
CACAAAAAGATTTTAATTTTATTATCGTTCAAACATTTGGCAATAAAGTTTCTTAAGATTGAATCCTGT
TGCCGGTCTTGCGATGATTATCATATAATTTCTGTTGAATTACGTTAAGCATGTAATAATTAACATGTA
ATGCATGACGTTATTTATGAGATGGGTTTTTATGATTAGAGTCCCGCAATTATACATTTAATACGCGAT
AGAAAACAAAATATAGCGCGCAAACTAGGATAAATTATCGCGCGCGGTGTCATCTATGTTACTAGATCT
CTAGAGTCTCAAGCTTGGCGCGCCCACGTGACTAGTGGCACTGGCCGTCGTTTTACAACGTCGTGACTG
GGAAAACCCTGGCGTTACCCAACTTAATCGCCTTGCAGCACATCCCCCTTTCGCCAGCTGGCGTAATAG
CGAAGAGGCCCGCACCGATCGCCCTTCCCAACAGTTGCGCAGCCTGAATGGCGAATGCTAGAGCAGCTT
GAGCTTGGATCAGATTGTCGTTTCCCGCCTTCAGTTTAAACTATCAGTGTTTGACAGGATATATTGGCG
GGTAAACCTAAGAGAAAAGAGCGTTTA
Construct 2879 from 2X35S prom to NOS term DNA
(SEQ ID NO: 39)
gtcaacatggtggagcacgacacacttgtctactccaaaaatatcaaagatacagtctcagaagaccaa
agggcaattgagacttttcaacaaagggtaatatccggaaacctcctcggattccattgcccagctatc
tgtcactttattgtgaagatagtggaaaaggaaggtggctcctacaaatgccatcattgcgataaagga
aaggccatcgttgaagatgcctctgccgacagtggtcccaaagatggacccccacccacgaggagcatc
gtggaaaaagaagacgttccaaccacgtcttcaaagcaagtggattgatgtgataacatggtggagcac
gacacacttgtctactccaaaaatatcaaagatacagtctcagaagaccaaagggcaattgagactttt
caacaaagggtaatatccggaaacctcctcggattccattgcccagctatctgtcactttattgtgaag
atagtggaaaaggaaggtggctcctacaaatgccatcattgcgataaaggaaaggccatcgttgaagat
gcctctgccgacagtggtcccaaagatggacccccacccacgaggagcatcgtggaaaaagaagacgtt
ccaaccacgtcttcaaagcaagtggattgatgtgatatctccactgacgtaagggatgacgcacaatcc
cactatccttcgcaagacccttcctctatataaggaagttcatttcatttggagaggtattaaaatctt
aataggttttgataaaagcgaacgtggggaaacccgaaccaaaccttcttctaaactctctctcatctc
tcttaaagcaaacttctctcttgtctttcttgcgtgagcgatcttcaacgttgtcagatcgtgcttcgg
caccagtacaatggcgaaaaacgttgcgattttcggcttattgttttctcttcttgtgttggttccttc
tcagatcttcgcggatcgaatctgcactgggataacatcttcaaactcacctcatgtggtcaaaacagc
tactcaaggggaggtcaatgtgactggcgtgataccactgacaacaacaccaacaaaatcttattttgc
aaatctcaaaggaacaaggaccagagggaaactatgcccggactgtctcaactgtacagatctggatgt
ggccttgggcaggccaatgtgtgtggggaccacaccttctgctaaagcttcaatactccatgaggtcag
acctgttacatccgggtgctttcctataatgcacgacagaacaaaaatcaggcaactacccaatcttct
cagaggatatgaaaagatcaggttatcaacccaaaacgttatcgatgcagaaaaagcaccaggaggacc
ctacagacttggaacctcaggatcttgccctaacgctaccagtaaaatcggattttttgcaacaatggc
ttgggctgtcccaaaggacaactacaaaaatgcaacgaacccacaaacagtggaagtaccatacatttg
tacagaaggggaagaccaaattactgtttgggggttccattcggataacaaaacccaaatgaagagcct
ctatggagactcaaatcctcaaaagttcacctcatctgctaatggagtaaccacgcattatgtttctca
gattggcgacttcccagatcaaacagaagacggaggactaccacaaagcggcagaattgttgttgatta
catggtgcaaaaacctgggaaaacaggaacaattgtctatcaaaggggtgttttgttgcctcaaaaggt
gtggtgcgcgagtggcaggagcaaagtaataaaagggtcattgcctttaattggtgaagcagattgcct
tcatgaagaatacggtggattaaacaaaagcaagccttactacacaggaaaacatgcaaaagccatagg
aaattgcccaatatgggtaaaaacacctttgaagcttgccaatggaaccaaatatagacctcctggtgg
aggatgggaaggaatgattgcaggttggcacggatacacatctcacggagcacatggagtggcagtggc
ggcagaccttaagagtacacaagaagctataaataagataacaaaaaatctcaattctttgagtgaact
agaagtaaagaaccttcaaagactaagtggtgccatggatgaactccacaacgaaatactcgagctgga
tgaaaaagtggatgatctcagagctgacactataagctcacaaatagaacttgcagtcttgctttccaa
cgaaggaataataaacagtgaagacgagcatctattggcacttgagagaaaactaaagaaaatgctggg
tccctctgctgtagacataggaaacggatgcttcgaaaccaaacacaaatgcaaccagacctgcttaga
caggatagctgctggcacctttaatgcaggagaattttctctccccacttttgattcattgaacattac
tgctgcatctttaaatgatgatggattggataactaccagattttggcgatctattcaactgtcgccag
ttcattggtactggtagtctccctgggggcaatcagtttctggatgtgctctaatgggtctctacagtg
tagaatatgtatttaaaggcctattttctttagtttgaatttactgttattcggtgtgcatttctatgt
ttggtgagcggttttctgtgctcagagtgtgtttattttatgtaatttaatttctttgtgagctcctgt
ttagcaggtcgtcccttcagcaaggacacaaaaagattttaattttattaaaaaaaaaaaaaaaaaaga
ccgggaattcgatatcaagcttatcgacctgcagatcgttcaaacatttggcaataaagtttcttaaga
ttgaatcctgttgccggtcttgcgatgattatcatataatttctgttgaattacgttaagcatgtaata
attaacatgtaatgcatgacgttatttatgagatgggtttttatgattagagtcccgcaattatacatt
taatacgcgatagaaaacaaaatatagcgcgcaaactaggataaattatcgcgcgcggtgtcatctatg
ttactagat
Influenza B HA2 consensus sequence (fusion peptide (FP) in italics,
TM/CT underlined)
(SEQ ID NO: 40)
GFFGAIAGFLEGGWEGMIAGWHGYTSHGAHGVAVAADLKSTQEAINKITKNLNSLSELEVKNL
QRLSGAMDELHNEIXELDEKVDDLRADTISSQIELAVLLSNEGIINSEDEHLLALERKLKKMLGPX
AVXIGNGCFETKHKCNQTCLDRIAAGTEXXGEFSLPTFDSLNITAASLNDDGLDNHTILLYYSTAA
SSLAVTLMXAIFXVYMVSRDNVSCSICL
Wherein
X (78) = L or I
X (129) = S or absent
X (132) = E or D
X (158) = N or D
X (159) A or D
X (204) = L or I
X (208) = I ov V
Influenza B HA2 fusion peptide and HA2 ectodomain consensus sequence with
modification at position 402 (X₁)
(SEQ ID NO: 41)
GFFGAIAGFLEGGWEGMIAGWHGYTSHGAHGVAVAADLKSTQEAINKITKNLNSLSEX₁EVKNL
QRLSGAMDELHNEIX₂ELDEKVDDLRADTISSQIELAVLLSNEGIINSEDEHLLALERKLKKMLGPX₃
AVX₄IGNGCFETKHKCNQTCLDRIAAGTFX₅X₆GEFSLPTFDSLNITAASLNDDGLDNHTI
Influenza B HA2 ectodomain consensus sequence with modification at position
402 (X₁)
(SEQ ID NO: 42)
YTSHGAHGVAVAADLKSTQEAINKITKNLNSLSEX₁EVKNLQRLSGAMDELHNEIX₂ELDEKV
DDLRADTISSQIELAVLLSNEGIINSEDEHLLALERKLKKMLGPX₃AVX₄IGNGCFETKHKCN
QTCLDRIAAGTFX₅X₆GEFSLPTFDSLNITAASLNDDGLDNHTI
Wherein:
X1 = I
X2 = L or I
X3 = S or absent
X4 = E or D
X5 = N or D
X6 = A or D
wt H5 A/Indonesia/5/2005 AA
(SEQ ID NO: 43)
DQICIGYHANNSTEQVDTIMEKNVTVTHAQDILEKTHNGKLCDLDGVKPLILRDCSVAGWLLGNPMCDE
FINVPEWSYIVEKANPTNDLCYPGSENDYEELKHLLSRINHFEKIQIIPKSSWSDHEASSGVSSACPYL
GSPSFFRNVVWLIKKNSTYPTIKKSYNNTNQEDLLVLWGIHHPNDAAEQTRLYQNPTTYISIGTSTLNQ
RLVPKIATRSKVNGQSGRMEFFWTILKPNDAINFESNGNFIAPEYAYKIVKKGDSAIMKSELEYGNCNT
KCQTPMGAINSSMPFHNIHPLTIGECPKYVKSNRLVLATGLRNSPQRESRRKKRGLFGAIAGFIEGGWQ
GMVDGWYGYHHSNEQGSGYAADKESTQKAIDGVTNKVNSIIDKMNTQFEAVGREENNLERRIENLNKKM
EDGELDVWTYNAELLVLMENERTLDFHDSNVKNLYDKVRLQLRDNAKELGNGCFEFYHKCDNECMESIR
NGTYNYPQYSEEARLKREEISGVKLESIGTYQILSIYSTVASSLALAIMMAGLSLWMCSNGSLQCRICI
All citations are hereby incorporated by reference.

The present invention has been described with regard to one or more embodiments. However, it will be apparent to persons skilled in the art that a number of variations and modifications can be made without departing from the scope of the invention as defined in the claims.

1. A modified influenza B virus hemagglutinin (HA) protein comprising a modified HA2 ectodomain, wherein the modified HA2 ectodomain comprises an amino acid sequence with at least one amino acid substitution compared to a parent HA2 ectodomain amino acid sequence, wherein the at least one substitution corresponds to amino acid position 402 in sequence alignment with reference sequence of SEQ ID NO: 1 (B/Washington/09/19 HA).

2. The modified influenza B HA protein of claim 1, wherein the parent HA2 ectodomain amino acid sequence is a wildtype amino acid sequence of an influenza B virus.

3. (canceled)

4. The modified influenza B HA protein of claim 1, wherein the substitution is to an isoleucine or a conserved substitution of isoleucine.

5. The modified influenza B HA protein claim 4, wherein the conserved substitution of isoleucine is methionine, phenylalanine or valine.

6. The modified influenza B HA protein of claim 1, wherein the sequences of the modified HA2 ectodomain comprises from 80% to 100% identity with the sequence of SEQ ID NO: 42.

7. The modified influenza B HA protein of claim 1, wherein the sequence of the influenza B HA protein comprises from 80% to 100% identity with the sequence of SEQ ID NO: 13, 17, 21, 25, 29, 33 or 37.

8. (canceled)

9. A nucleic acid comprising a nucleotide sequence encoding the modified influenza HA protein of claim 1.

10. A virus-like particle (VLP) comprising the modified influenza B HA protein of claim 1.

11. A method of producing a modified influenza B HA protein in a non-human host or host cell comprising:
a) introducing the nucleic acid of claim 9 into the non-human host or host cell, or providing the non-human host or host cell comprising the nucleic acid of claim 9, and b) incubating the non-human host or host cell under conditions that permit the expression of the nucleic acid, thereby producing the modified influenza B HA protein.

12.-14. (canceled)

15. A method of producing an influenza virus like particle (VLP) in a non-human host or host cell, comprising:
a) providing the non-human host or host cell comprising the nucleic acid of claim 9; or
introducing into the non-human host or host cell the nucleic acid of claim 9; and
b) incubating the non-human host or host cell under conditions that permit expression of the modified influenza B HA protein encoded by the nucleic acid, thereby producing the VLP.

16.-19. (canceled)

20. A method of producing an antibody or antibody fragment comprising, administering the VLP of claim 10 to a subject, or a host animal, thereby producing the antibody or the antibody fragment.

21. An antibody produced by the method of claim 20.

22. A host or host cell comprising the nucleic acid of claim 9.

23. A composition for inducing an immune response comprising, an effective dose of the VLP of claim 10, and a pharmaceutically acceptable carrier, adjuvant, vehicle or excipient.

24. A vaccine for inducing an immune response, the vaccine comprising an effective dose of the modified influenza B HA protein of claim 1.

25. The vaccine of claim 24, further comprising an adjuvant.

26. A method for inducing an immune response to an influenza infection in a subject, the method comprising administering the VLP of claim 10 to the subject.

27. (canceled)

28. The method of claim 11, wherein the non-human host or host cell comprises a plant, portion of a plant, a plant cell, a fungi, a fungi cell, an insect, an insect cell, an animal or an animal cell.

29. A multivalent immunogenic composition comprising two or more than two types of VLP, wherein at least one type of VLP comprises the modified influenza B HA of claim 1.

30.-32. (canceled)

33. A quadrivalent immunogenic composition comprising a first type of VLP comprising the modified influenza B HA of claim 1, a second type of VLP comprising the modified influenza B HA of claim 1, a third type of VLP comprising influenza A HA and a fourth type of VLP comprising influenza A HA, wherein the first type VLP comprises modified B HA that are derived from a different influenza B lineage than the modified B HA of the second type of VLP.
